(19) United States
(12) Patent Application Publication
(10) Pub. No.: US 2026/0028390 A1
(43) Pub. Date: Jan. 29, 2026

(54) ALPHA1-ANTITRYPSIN FOR USE IN THE TREATMENT OF DISEASES OR DISORDERS OF THE NERVOUS SYSTEM SUCH AS CHRONIC INFLAMMATORY DEMYELINATING POLYNEUROPATHY

(71) Applicant: Ageronix SA, Carouge GE (CH)

(72) Inventor: Nikolay ZHUKOVSKY, Carouge GE (CH)

(21) Appl. No.: 18/862,432

(22) PCT Filed: Jun. 22, 2023
(86) PCT No.: PCT/EP2023/067056

§ 371 Date: Nov. 1, 2024

(30) Foreign Application Priority Data
Jun. 22, 2022 (EP) 22180584.9
Oct. 19, 2022 (EP) 22202628.8
Feb. 10, 2023 (EP) 23156173.9

(51) Int. Cl. C07K 14/81; A61K 38/00; A61P 25/02; A61P 29/00; A61P 37/06

(52) U.S. Cl. CPC C07K 14/8125; A61P 25/02; A61P 29/00; A61P 37/06; A61K 38/00

(57) ABSTRACT

The disclosure relates to alpha1-antitrypsin (AAT) protein, a variant, an isoform and/or a fragment thereof and/or a corresponding nucleic acid sequence for use in the treatment of diseases or disorders of the nervous system such as chronic inflammatory demyelinating polyneuropathy. The disclosure further relates to combinations with IgG antibodies, such as immunoglobulin therapy, for use in these diseases or disorders.

### CROSS-REFERENCE TO RELATED APPLICATIONS

This application is a Section 371 of International Application No. PCT/EP2023/067056, filed Jun. 22, 2023, which was published in the English language on Dec. 28, 2023 under International Publication No. WO 2023/247736 A1, which claims priority under 35 U.S.C. § 119(b) to European Application No. 22180584.9, filed Jun. 22, 2022, European Application No. 22202628.8, filed Oct. 19, 2022, and European Application No. 23156173.9, filed Feb. 10, 2023, the disclosures of which are incorporated herein by reference in their entireties.

### REFERENCE TO SEQUENCE LISTING SUBMITTED ELECTRONICALLY

This application contains a sequence listing, which is submitted electronically as an XML formatted sequence listing with a file name “209282_43US_Sequence_Listing”, creation date of Nov. 1, 2024, and having a size of 8,718 bytes. The sequence listing submitted electronically is part of the specification and is herein incorporated by reference in its entirety.

### BACKGROUND OF THE INVENTION

The invention relates to alpha1-antitrypsin (AAT) protein, a variant, an isoform and/or a fragment thereof and/or a corresponding nucleic acid sequence for use in the treatment of diseases or disorders of the nervous system such as chronic inflammatory demyelinating polyneuropathy. The invention further relates to combinations with IgG antibodies, such as immunoglobulin therapy, for use in these diseases or disorders.

Diseases or disorders of the nervous system are diseases or disorders which can dramatically affect the peripheral and/or central nervous system (PNS/CNS). In the last decade, neuroinflammation has become more and more central in our understanding of neurological disorders. Inflammation per se may directly or indirectly trigger the disease but it does undoubtedly contribute to the pathogenesis of the disease throughout the peripheral (PNS) and central nervous systems (CNS).

Peripheral neuropathies constitute a highly diverse group of disorders with the major component being damages to the myelin sheaths, either after its abnormal development (dysmyelination) in the inherited forms (CMT1A-F and -X) or direct in the acquired ones such as chronic inflammatory demyelinating polyneuropathy.

Chronic inflammatory demyelinating polyneuropathy (CIDP) is an autoimmune disease that targets myelin sheaths, specifically in the peripheral nerves, and causes progressive weakness and sensory loss. Swelling of nerve roots is also a characteristic of the disease. Although it can occur at any age and in both genders, CIDP is more common in young adults, and it is more common in men than women.

Untreated, CIDP is characterized by accumulating disability that requires physical and occupational therapy, orthotic devices and long-term treatment. Early intervention can prevent permanent damage and disability. Current methods of treatment for CIDP include administration of corticosteroids, such as prednisone, which may be prescribed alone or in combination with immunosuppressant drugs. Immunosuppressant drugs may also be given in the absence of a steroid.

Myelin is produced by Schwann cells (SCs) in the PNS and is crucial for proper transmission of the electric impulse in the nerves. In the intricate neuron/glia cross-communication that is required for proper myelin regulation (Rao and Pearse 2016), several diverse signaling pathways are involved, which include growth factors, integrins and cell adhesion molecules but more importantly, the pivotal neuregulin 1 type III (NRG1-III) that signals through the ERBB2/3 receptors and its proteolytic sheddase modulator, the tumor necrosis factor-a-converting enzyme, TACE (also known as ADAM17). TACE/ADAM17, is a transmembrane protein that includes an extracellular zinc-dependent protease domain. In the context of the PNS, ADAM17 is known for its inhibitory effect on SCs mediated myelination by cleaving NRG1-III in the epidermal growth factor domain in a ligand independent manner (La Marca, R., 2011, Nat Neurosci 14(7): 857-865.). Conflicting evidence has been reported in the literature with respect to the role of the human protease alpha-1-Antitrypsin (AAT), specifically, in 2013 AAT was shown not to interact with TACE (van′t Wout E. F. et al., 2014, Hum Mol Genet.; 23(4):929-4) in contrast to an earlier report in 2010 that claimed AAT does indeed interact with TACE and inhibits its activity in a dose dependent manner (Bergin, D. A. et al., 2010, J Clin Invest 120(12): 4236-4250.).

The biological common trait of many PNS and CNS neurodegenerative diseases is a sustained and acute inflammatory response due to cytokine release orchestrated in feed-forward loops (also called “cytokine storm”). Therefore, dampening of the inflammatory reaction stands as a central target of therapeutical strategies. However, the subtleties of inflammatory mechanisms underlying its multiple mediators are not fully understood.

Thus, there is a need for improved therapies for diseases or disorders of the nervous system, in particular chronic inflammatory demyelinating polyneuropathy.

### BRIEF SUMMARY OF THE INVENTION

The above technical problem is solved by the embodiments disclosed herein and as defined in the claims.

Accordingly, the invention relates to, inter alia, the following embodiments:
1. A pharmaceutical product for use in the treatment of an inflammatory disease or disorder, the pharmaceutical product comprising:
   a) alpha1-antitrypsin (AAT) protein, a variant, an isoform and/or a fragment thereof, wherein said variant, isoform and/or fragment has ADAM17 inhibitory activity or a small molecule having ADAM17 inhibitory activity; and/or
   b) a nucleic acid encoding AAT, a variant, an isoform and/or a fragment thereof, wherein said variant, isoform and/or fragment has inhibitory activity.
2. The pharmaceutical product for use of embodiment 1, wherein the inflammatory disease or disorder is an autoimmune inflammatory disease.
3. The pharmaceutical product for use of embodiment 1 or 2, wherein the inflammatory disease or disorder is an inflammatory disease or disorder of the nervous system, preferably wherein the inflammatory disease or disorder is neuropathic pain caused by an inflammatory disease or disorder of the nervous system.
4. The pharmaceutical product for use of any one of embodiments 1 to 3, wherein the inflammatory disease is chronic inflammatory demyelinating polyneuropathy.
5. The pharmaceutical product for use of embodiments 1 or 2, wherein the inflammatory disease is complex regional pain syndrome.
6. The pharmaceutical product for use of any one of embodiments 1 to 3, wherein the inflammatory disease or disorder is inflammatory pain.
7. A kit of parts for use in the treatment of a disease or disorder of the nervous system, the kit comprising:
   i) a) alpha1-antitrypsin (AAT) protein, a variant, an isoform and/or a fragment thereof, wherein said variant, isoform and/or fragment has inhibitory activity or a small molecule having ADAM17 inhibitory activity; and/or
      b) a nucleic acid encoding AAT, a variant, an isoform and/or a fragment thereof, wherein said variant, isoform and/or fragment has inhibitory activity; and
   ii) a plurality of IgG antibodies, isoforms thereof, fragments thereof, and/or IgG variants.
8. A pharmaceutical composition for use in the treatment of a disease or disorder of the nervous system, the composition comprising:
   i) a) alpha1-antitrypsin (AAT) protein, a variant, an isoform and/or a fragment thereof, wherein said variant, isoform and/or fragment has inhibitory activity or a small molecule having ADAM17 inhibitory activity; and/or
      b) a nucleic acid encoding AAT, a variant, an isoform and/or a fragment thereof, wherein said variant, isoform and/or fragment has inhibitory activity; and
   ii) a plurality of IgG antibodies, IgG variants, isoforms and/or fragments thereof; and
   iii) at least one pharmaceutically acceptable carrier.
9. A method of treatment comprising administering an effective amount of a pharmaceutical composition to a subject, wherein the subject is suffering from a disease or disorder of the nervous system and wherein the subject is undergoing a therapy comprising administration of a plurality of IgG antibodies, isoforms thereof, fragments thereof, and/or IgG variants, the pharmaceutical composition comprising
   a) AAT protein, a variant, an isoform and/or a fragment thereof, wherein said variant, isoform and/or fragment has inhibitory activity or a small molecule having ADAM17 inhibitory activity; and/or
   b) a nucleic acid encoding AAT, a variant, an isoform and/or a fragment thereof, wherein said variant, isoform and/or fragment has inhibitory activity.
10. A method of treatment comprising administering an effective amount of a pharmaceutical compound comprising a plurality of IgG antibodies, isoforms thereof, fragments thereof, and/or IgG variants to a subject, wherein the subject is suffering from a disease or disorder of the nervous system and wherein the subject is undergoing a therapy comprising administration of
   a) AAT protein, a variant, an isoform and/or a fragment thereof, wherein said variant, isoform and/or fragment has inhibitory activity or a small molecule having ADAM17 inhibitory activity; and/or
   b) a nucleic acid encoding AAT, a variant, an isoform and/or a fragment thereof, wherein said variant, isoform and/or fragment has inhibitory activity.
11. The kit of parts for use of embodiment 7, the pharmaceutical composition for use of embodiment 8, the method of treatment of embodiment 9 or 10, wherein the disease or disorder of the nervous system is pain caused by a disease or disorder of the nervous system, preferably chronic pain caused by a disease or disorder of the nervous system.
12. The kit of parts for use of embodiment 7 or 11, the pharmaceutical composition for use of embodiment 8 or 11, the method of treatment of embodiment 9 to 11, wherein the disease or disorder of the nervous system is an autoimmune disease or disorder of the nervous system.
13. The kit of parts for use of embodiment 12, the pharmaceutical composition for use of embodiment 12, the method of treatment of embodiment 12, wherein the disease or disorder of the nervous system is chronic inflammatory demyelinating polyneuropathy.
14. The pharmaceutical product for use of embodiment 4 or 6, the kit of parts for use of embodiment 13, the pharmaceutical composition for use of embodiment 13, the method of treatment of embodiment 13, wherein a subject to be treated has at least one symptom of chronic inflammatory demyelinating polyneuropathy or a history of at least one symptom of chronic inflammatory demyelinating polyneuropathy.
15. The kit of parts for use of any one of the embodiments 7, 11 to 14, the pharmaceutical composition for use of any one of the embodiments 8, 11 to 14, the method of treatment of any one of the embodiments 9 to 14, wherein the plurality of IgG antibodies, isoforms thereof, fragments thereof, and/or IgG variants are plasma derived IgG antibodies, isoforms thereof, fragments thereof, and/or IgG variants.
16. The kit of parts for use of any one of the embodiments 7, 11 to 14, the pharmaceutical composition for use of any one of the embodiments 8, 11 to 14, the method of treatment of any one of the embodiments 9 to 14, wherein the plurality of IgG antibodies, isoforms thereof, fragments thereof, and/or IgG variants are recombinant IgG antibodies, isoforms thereof, fragments thereof, and/or IgG variants.
17. The kit of parts for use of any one of the embodiments 7, 11 to 16, the pharmaceutical composition for use of any one of the embodiments 8, 11 to 16, the method of treatment of any one of the embodiments 9 to 16, wherein the plasma derived IgG antibodies, isoforms thereof, fragments thereof, and/or IgG variants are formulated for intravenous administration.
18. The kit of parts for use of any one of the embodiments 7, 11 to 16, the pharmaceutical composition for use of any one of the embodiments 8, 11 to 16, the method of treatment of any one of the embodiments 9 to 16, wherein the plasma derived IgG antibodies, isoforms thereof, fragments thereof, and/or IgG variants are formulated for subcutaneous administration.
19. The pharmaceutical product for use of any one of the embodiments 1 to 6, the kit of parts for use of any one of the embodiments 7, 11 to 18, the pharmaceutical composition for use of any one of the embodiments 8, 11 to 18, the method of treatment of any one of the embodiments 9 to 18, wherein the AAT protein is recombinant AAT.
20. The pharmaceutical product for use of any one of the embodiments 1 to 6, the kit of parts for use of any one of the embodiments 7, 11 to 18, the pharmaceutical composition for use of any one of the embodiments 8, 11 to 18, the method of treatment of any one of the embodiments 9 to 18, wherein the AAT protein is plasma derived AAT.
21. The pharmaceutical product for use of any one of the embodiments 1 to 6, 19 or 20, the kit of parts for use of any one of the embodiments 7, 11 to 20, the pharmaceutical composition for use of any one of the embodiments 8, 11 to 20, the method of treatment of any one of the embodiments 9 to 20, wherein
   a) alpha1-antitrypsin (AAT) protein, a variant, an isoform and/or a fragment thereof, wherein said variant, isoform and/or fragment has inhibitory activity or a small molecule having ADAM17 inhibitory activity; and/or
   b) a nucleic acid encoding AAT, a variant, an isoform and/or a fragment thereof, wherein said variant, isoform and/or fragment has inhibitory activity.
is/are formulated for intravenous administration.
22. The pharmaceutical product for use of any one of the embodiments 1 to 6, 19 or 20, the kit of parts for use of any one of the embodiments 7, 11 to 20, the pharmaceutical composition for use of any one of the embodiments 8, 11 to 20, the method of treatment of any one of the embodiments 9 to 20, wherein
   a) alpha1-antitrypsin (AAT) protein, a variant, an isoform and/or a fragment thereof, wherein said variant, isoform and/or fragment has inhibitory activity or a small molecule having ADAM17 inhibitory activity; and/or
   b) a nucleic acid encoding AAT, a variant, an isoform and/or a fragment thereof, wherein said variant, isoform and/or fragment has inhibitory activity.
is/are formulated for subcutaneous administration.

Accordingly, in one embodiment, the invention relates to a pharmaceutical product for use in the treatment of an inflammatory disease or disorder, the pharmaceutical product comprising: a) alpha1-antitrypsin (AAT) protein, a variant, an isoform and/or a fragment thereof, wherein said variant, isoform and/or fragment has inhibitory activity or a small molecule having ADAM17 inhibitory activity; and/or b) a nucleic acid encoding AAT, a variant, an isoform and/or a fragment thereof, wherein said variant, isoform and/or fragment has inhibitory activity.

The term “inflammatory disease or disorder”, as used herein, refers to a disease, a disorder or a condition that is characterized by increased inflammation in a tissue, organ or the system compared to a corresponding tissue, organ or system of a healthy reference subject. Inflammation is characterized by a dysregulation of inflammation markers and/or increased immune cell infiltration, activation, proliferation, and/or differentiation in the blood and/or the tissue. An inflammation marker is a marker that is indicative for inflammation in a subject. In certain aspects the inflammatory marker described herein is a marker selected from the group of CRP, erythrocyte sedimentation rate (ESR), and procalcitonin (PCT), Interleukin (e.g., IL-1, IL-2, IL-3, IL-4, IL-5, IL-6, IL-7, IL-8, IL-9, IL-10, IL-11, IL-12, IL-13, IL-14, IL-15, IL-16, IL-17, IL-18, IL-19, IL-20, IL-21, IL-22, IL-23, IL-24, IL-25, IL-26, IL-27, IL-28, IL-29, IL-30, IL-31, IL-33, IL-32, IL-33, IL-35 or IL-36) Tumor necrosis factor (e.g., TNF-alpha, TNF-beta), Interferon (e.g., interferon gamma) MIP-I, MCP-I, RANTES, other chemokines and/or other cytokines. An inflammatory marker may also be detectable indirectly, e.g., by detection of an inhibitory factor of an inflammatory marker (e.g., binding factor and/or antagonist). In some aspects, the inflammatory marker is measured in cells involved in inflammation, in cells affected by cells involved in inflammation, in the cerebrospinal fluid, and/or in the blood. In some aspects, the inflammation marker is indicative for immune cell infiltration, activation, proliferation and/or differentiation. Detection of the inflammation marker or the ratio of two or more inflammation markers is detected outside the normal range. The normal range of inflammation markers and whether a marker(ratio) has to be below or above a threshold to be indicative for inflammation is known to the person skilled in the art. In some aspects, the gene expression level, the RNA transcript level, the protein expression level, the protein activity level and/or the enzymatic activity level of at least one inflammation marker is detected. In some aspects at least one inflammation marker is detected quantitatively and/or qualitatively to determine the inflammatory disease or disorder in a subject in need of treatment and/or prevention.

In some aspects, the inflammatory disease or disorder described herein is characterized by acute inflammation, that is the duration of inflammation symptoms typically takes from about a few minutes (e.g., 2, 5, 10, 15, 30, 45 minutes) to a few days (e.g., 2, 3, 5, 7, 10 or 14 days). In some aspects, the inflammatory disease or disorder is characterized by chronic inflammation, that is the duration of symptoms of inflammation typically take at least about a few days (e.g., 2, 3, 5, 7, 10 or 14 days) or the symptoms of inflammation reoccur at least once (e.g., once or more times, twice or more times or three or more times). In some aspects, the inflammatory disease or disorder is characterized by chronic low-grade inflammation. Chronic low-grade inflammation can occur in the absence of clinical symptoms. In certain embodiments, the inflammatory disease described herein is an inflammatory disease characterized by increase inflammation affecting the Schwann Cell (SC) phenotype or health. In certain embodiments, the inflammatory disease described herein is an inflammatory disease characterized by an increase of inflammatory microglia and/or an increase in microglia inflammatory phenotypes.

The inventors found that AAT is able to inhibit TACE/ADAM17 in a dose-dependent manner which, without being bound by theory, rescues myelin production by SCs, and thus subsequently prevents, or slowing, and/or reverses the progression of inflammatory diseases. Furthermore, alpha1-antitrypsin (AAT) protein and variant thereof attenuate the reactive oxygen species (ROS) response of SCs when stimulated with the pro-inflammatory cytokine TNFα (TNF-. This indicates that AAT and its derivatives exert a novel therapeutic effect by acting directly on Schwann Cells (SCs) and rescuing these cells from their inflamed state back to their natural wild type state, e.g., rescuing SCs ability to restore myelination around axons. As such, this anti-inflammatory effect on SCs in the PNS or microglia in the CNS is surprisingly effective in the treatment of inflammatory diseases involving these cells.

Accordingly, the invention is at least in part based on the anti-inflammatory effect of AAT and variants, isoforms and/or fragments thereof.

In certain embodiments, the invention relates to the pharmaceutical product for use of the invention, wherein the inflammatory disease or disorder is an autoimmune inflammatory disease.

The term “autoimmune inflammatory disease”, as used herein refers to a group of diseases or disorders in which tissue injury is associated with a humoral and/or cell-mediated immune response to body constituents or, in a broader sense, an immune response to self. The pathological immune response may be systemic or organ specific. In certain embodiments, autoimmune disease described herein is a disease selected from the group consisting of multiple sclerosis, myasthenia gravis, Pernicious anemia, arthritis, Sjögren syndrome, systemic lupus erythematosus, complex regional pain syndrome and type I diabetes.

In certain embodiments, the invention relates to the pharmaceutical product for use of the invention, wherein the inflammatory disease or disorder is an inflammatory disease or disorder of the nervous system. In certain embodiments, the invention relates to the pharmaceutical product for use of the invention, wherein the inflammatory disease or disorder is neuropathic pain.

In certain embodiments, the invention relates to the kit of parts, the pharmaceutical composition and the method of treatment for use of the invention, wherein the disease or disorder of the nervous system is neuropathic pain.

In certain embodiments, the invention relates to the pharmaceutical product for use of the invention, wherein the inflammatory disease or disorder is neuropathic pain caused by an inflammatory disease or disorder of the nervous system.

The term “inflammatory disease or disorder of the nervous system”, as used herein, refers to a disease, a disorder or a condition that is characterized by increased inflammation in the nervous system compared to a healthy reference subject.

In certain embodiments, the invention relates to the pharmaceutical product for use of the invention, wherein the inflammatory disease or disorder, preferably the inflammatory disease or disorder of the nervous system, is selected from the group consisting of inflammatory pain, neuropathic pain, autoimmune inflammatory disease, complex regional pain syndrome and chronic inflammatory demyelinating polyneuropathy. In certain embodiments, the invention relates to the pharmaceutical product for use of the invention, wherein the inflammatory disease or disorder, preferably the inflammatory disease or disorder of the nervous system, is selected from the group consisting of autoimmune inflammatory disease, complex regional pain syndrome and chronic inflammatory demyelinating polyneuropathy. In certain embodiments, the invention relates to the pharmaceutical product for use of the invention, wherein the inflammatory disease or disorder, preferably the inflammatory disease or disorder of the nervous system, is selected from the group consisting of complex regional pain syndrome and chronic inflammatory demyelinating polyneuropathy.

The term “neuropathic pain” as used herein refers to pain caused by damage or disease affecting the somatosensory system, preferably the pain is caused by an inflammatory disease or disorder. The term “neuropathic pain caused by an inflammatory disease or disorder of the nervous system”, as used herein refers to pain induced by damage to the nerves, wherein the damage is caused by an inflammatory disease or disorder. In certain embodiments, the pain described herein is considered to be “caused” by a disease, if the disease and the pain are present, and the disease is known to have the pain as a symptom, preferably wherein the disease is the most likely cause of the pain of a subject.

In certain embodiments, the invention relates to the pharmaceutical product for use of the invention, wherein the inflammatory disease is complex regional pain syndrome.

In certain embodiments, the invention relates to the kit of parts, the pharmaceutical composition and the method of treatment for use of the invention, wherein the disease or disorder of the nervous system is complex regional pain syndrome.

The term “complex regional pain syndrome”, as used herein refers to a syndrome that is characterized by excess and prolonged pain and inflammation that follows an injury, typically in injury to an arm or leg.

In certain embodiments, the invention relates to the pharmaceutical product for use of the invention, wherein the inflammatory disease or disorder is inflammatory pain.

In certain embodiments, the invention relates to the kit of parts, the pharmaceutical composition and the method of treatment for use of the invention, wherein the disease or disorder of the nervous system is inflammatory pain.

The term “inflammatory pain”, as used herein is pain that occurs in response to tissue damage and inflammation. As such, inflammatory pain is pain that is neither nociceptive pain nor neuropathic pain. In certain embodiments, the inflammatory pain described herein is caused by an inflammatory disease.

The term “pain”, as described herein, refers to the term as used in the art. In certain embodiments, the pain described herein is chronic pain such that it occurs for longer than 2, longer than 4, longer than 6, longer than 8 or longer than 12 weeks. In certain embodiments, the pain described herein is breakthrough pain in a subject with chronic pain or in a subject under pain reducing therapy (such as pharmacological pain treatment) or a subject with chronic pain under pain reducing therapy. In certain embodiments, the pain described herein occurs without causal history of injury or operation. In certain embodiments, the pain described herein is self-reported pain. The numeric rating scales (NRS) is the most commonly used pain scale for scaling pain on a scale of 0 o 10. In certain embodiments, the pain described herein is pain rated larger than 1, larger than 2, lager than 3, larger than 4, larger than 5, larger than 6, larger than 7, larger than 8 or larger than 9 on NRS.

In certain embodiments, the invention relates to a pharmaceutical product for use in the treatment of pain caused by a disease or disorder of the nervous system, the pharmaceutical product comprising: a) alpha1-antitrypsin (AAT) protein, a variant, an isoform and/or a fragment thereof, wherein said variant, isoform and/or fragment has inhibitory activity or a small molecule having ADAM17 inhibitory activity; and/or b) a nucleic acid encoding AAT, a variant, an isoform and/or a fragment thereof, wherein said variant, isoform and/or fragment has inhibitory activity.

Oxidative stress is well established to be associated with neuropathic pain. The inventors found that oxidative stress is downregulated by AAT and its peptide derivatives, in particular in SCs which reduces pain.

Accordingly, the invention is at least in part based the effect of AAT and variants, isoforms and/or fragments thereof on oxidative stress.

In certain embodiments, the invention relates to a pharmaceutical product for use in the treatment of chronic inflammatory demyelinating polyneuropathy, the pharmaceutical product comprising: a) alpha1-antitrypsin (AAT) protein, a variant, an isoform and/or a fragment thereof, wherein said variant, isoform and/or fragment has inhibitory activity or a small molecule having ADAM17 inhibitory activity; and/or b) a nucleic acid encoding AAT, a variant, an isoform and/or a fragment thereof, wherein said variant, isoform and/or fragment has inhibitory activity.

As such the pharmaceutical product described herein may comprise alpha1-antitrypsin (AAT) protein, a variant of AAT having ADAM17 inhibitory activity, an isoform of AAT having ADAM17 inhibitory activity or a fragment of AAT having ADAM17 inhibitory activity or any combination thereof. Additionally or alternatively may comprise a small molecule having ADAM17 inhibitory activity.

The terms “peptide”, “protein”, “polypeptide”, “polypeptidic” and “peptidic” are used herein interchangeably to designate a series of amino acid residues connected to the other by peptide bonds between the alpha-amino and carboxy groups of adjacent residues.

The term “Alphal-Antitrypsin protein” or “AAT”, as used herein refers to a protease inhibitor belonging to the serpin superfamily. Preferably AAT is mammalian AAT, more preferably human AAT. In humans AAT is encoded by the SERPINA1 gene. The term AAT includes natural variants, post-translational modified AAT and isoforms of AAT, preferably produced by alternative splicing. Sequences of AAT of different species and origin can be found in protein and nucleic acid databases, such as UniProt, Genbank, DDBJ and EMBL. In a very preferred embodiment, the term “AAT” includes variants as disclosed in protein and nucleic acid databases. In a very preferred embodiment, the term “AAT” refers to a protein with an amino acid sequence as defined by the SEQ ID NO: 1 or a nucleotide sequence encoding a protein with an amino acid sequence as defined by the SEQ ID NO: 1. In some embodiments, the AAT described herein is a protein, peptide or polypeptide. AAT protein can be obtained by isolation from blood (e.g. human blood) or can be produced recombinantly.

The term “variant” refers to a protein, peptide or polypeptide having an amino acid sequence that differ to some extent from the AAT native sequence peptide, preferably SEQ ID NO: 1, that is an amino acid sequence that vary from the AAT native sequence, preferably SEQ ID NO: 1, by amino acid substitutions, whereby one or more amino acids are substituted by another with same characteristics and conformational roles. Preferably, a variant described herein is at least 80%, 81%, 82%, 83%, 84%, 85%, 86%, 87%, 88%, 89%, 90%, 91%, 92%, 93%, 94%, 95%, 96%, 97%, 98%, or 99% homologous to amino acids of SEQ ID NO: 1. The amino acid sequence variants can have substitutions, deletions, and/or insertions at certain positions within the amino acid sequence of the native amino acid sequence, e.g. at the N- or C-terminal sequence or within the amino acid sequence. Substitutions can also be conservative, in this case, the conservative amino acid substitutions are herein defined as exchanges within one of the following five groups:
I. Small aliphatic, nonpolar or slightly polar residues: Ala, Ser, Thr, Pro, Gly
II. Polar, positively charged residues: His, Arg, Lys
III. Polar, negatively charged residues; and their amides: Asp, Asn, Glu, Gln
IV. Large, aromatic residues: Phe, Tyr, Trp
V. Large, aliphatic, nonpolar residues: Met, Leu, Ile, Val, Cys.

In some embodiments, the AAT variant described herein is an AAT variant having protease inhibitory activity, preferably having a disintegrin and metalloprotease 17 (ADAM17) inhibitory activity. The “disintegrin and metalloprotease 17 (ADAM17) inhibitory activity” is preferably measured by the Recombinant Human ADAM-17 kit (Recombinant Human TACE/ADAM17 Protein, CF: 930-ADB-010 and Mca-PLAQAV-Dpa-RSSSR-NH2 Fluorogenic Peptide Substrate: ES003; R&D Systems, preferably under the conditions described in example 22). The “disintegrin and metalloprotease 17 (ADAM17) inhibitory activity” is at least about 30% ADAM17 activity reduction at 200 uM, preferably at least about 30% ADAM17 activity reduction at 100 uM and/or at least about 45% ADAM17 activity reduction at 200 uM, more preferably at least about 45% ADAM17 activity reduction at 15 uM. In these embodiments the concentration in uM may be the concentration of the AAT variant. In certain embodiments, wherein the AAT variant described herein is Ac-VKFNKPFVFLNleIEQNTK-NH2. In certain embodiments, the AAT variant described herein is a sequence related to Ac-VKFNKPFVFLNleIEQNTK-NH2 that is a sequence having not more than 5, not more than 4 not more than 3 or not more than one amino acid substitution(s) compared to Ac-VKFNKPFVFLNleIEQNTK-NH2, preferably wherein the amino acid substitution(s) are conservative amino acid substitutions. The sequence related to Ac-VKFNKPFVFLNleIEQNTK-NH2 may further comprise one or more terminal insertion(s). In certain embodiments, preferably in embodiments wherein the AAT variant described herein is Ac-VKFNKPFVFLNleIEQNTK-NH2 or a sequence related to Ac-VKFNKPFVFLNleIEQNTK-NH2, the AAT variant is not used in the treatment of acute nociceptive, inflammatory, and neuropathic pain. In certain embodiments, the Ac-VKFNKPFVFLNleIEQNTK-NH2 or a sequence related to Ac-VKFNKPFVFLNleIEQNTK-NH2 described herein is used in the treatment of chronic inflammatory demyelinating polyneuropathy or a symptom thereof.

As used herein, an “isoform” of an AAT protein, peptide or polypeptide of the invention refers to a splice variant resulting from alternative splicing of the AAT mRNA. In some embodiments, the AAT isoform described herein is an AAT isoform having ADAM17 inhibitory activity, preferably having ADAM17 inhibitory activity, more preferably having a disintegrin and metalloprotease 17 (ADAM17) inhibitory activity.

As used herein, a “fragment” of an AAT protein, peptide or polypeptide of the invention refers to a sequence containing less amino acids in length than the AAT protein, peptide or polypeptide of the invention, in particular less amino acids than the sequence of AAT as set forth in SEQ ID NO:1. The fragment is preferably a functional fragment, e.g. a fragment with the same biological activities as the AAT protein as set forth in SEQ ID NO:1. The functional fragment is preferably derived from the AAT protein as set forth in SEQ ID NO:1. Any AAT fragment can be used as long as it exhibits the same properties, i.e. is biologically active, as the native AAT sequence from which it derives. In some embodiments, the AAT fragment described herein is an AAT fragment having protease inhibitory activity, preferably having ADAM17 inhibitory activity, more preferably having a disintegrin and metalloprotease 17 (ADAM17) inhibitory activity.

Preferably, the (functional) fragment shares about 5 consecutive amino-acids, at least about 7 consecutive amino-acids, at least about 15 consecutive amino-acids, at least about 20 consecutive amino-acids, at least about 25 consecutive amino-acids, at least about 20 consecutive amino-acids, at least about 30 consecutive amino-acids, at least about 35 consecutive amino-acids, at least about 40 consecutive amino-acids, at least about 45 consecutive amino-acids, at least about 50 consecutive amino-acids, at least about 55 consecutive amino-acids, at least about 60 consecutive amino-acids, at least about 100 consecutive amino-acids, at least about 150 consecutive amino-acids, at least about 200 consecutive amino-acids, at least about 300 consecutive amino-acids, or more of the native human AAT amino acid sequence as set forth in SEQ ID NO: 1. In some embodiments, the (functional) fragment described herein, comprises an expression optimized signal protein. In certain embodiments, the fragment described herein is derived from the C-terminus of AAT, as such the fragment shares about 5 consecutive amino-acids, at least about 7 consecutive amino-acids, at least about 15 consecutive amino-acids, at least about 20 consecutive amino-acids, at least about 25 consecutive amino-acids, at least about 20 consecutive amino-acids, at least about 30 consecutive amino-acids, at least about 35 consecutive amino-acids, at least about 40 consecutive amino-acids, at least about 45 consecutive amino-acids, at least about 50 consecutive amino-acids, at least about 55 consecutive amino-acids, at least about 60 consecutive amino-acids, at least about 100 consecutive amino-acids, at least about 150 consecutive amino-acids, or more of the native human AAT amino acid sequence as set forth in SEQ ID NO:2. In a very preferred embodiment, the fragment is derived from the C-terminus of AAT, as such the fragment shares about at least about 200 or more of the native human AAT amino acid sequence as set forth in SEQ ID NO:2. in a very preferred embodiment, the fragment has the sequence of SEQ ID NO: 2.

As used herein, a “small molecule” having ADAM17 inhibitory activity refers to a molecule having a molecular weight of less than 1500 kD, less than 1400 kD, less than 1300 kD, less than 1200 kD, less than 1100 kD, less than 1000 kD, less than 900 kD, less than 800 kD, less than 700 kD, less than 600 kD, less than 500 kD or less than 400 kD. In certain embodiments, the small molecule described herein is a peptide having a molecular weight of between 1500 kD and 500 kD, preferably between 1300 kD and 600 kD, more preferably between 1100 kD and 700 kD. Examples of such peptides are given as peptide 9 and peptide 8 in example 22. In certain embodiments, the small molecule is a variant of peptide 9 and peptide 8, wherein one or two amino acids are replaced, inserted or removed, preferably replaced by a conservative amino acid substitution. In certain embodiments, the small molecule described herein is a molecule having a molecular weight of between 1000 kD and 50 kD, preferably between 600 kD and 100 kD, more preferably between 400 kD and 200 kD. Examples of such a small molecule is provided in example 22 (peptidomimetic 14). The skilled person is aware how to identify further small molecules for use of the invention, e.g., by following the teaching of Lior, Yotam, et al. (European Journal of Medicinal Chemistry 228 (2022): 113969) and screening for ADAM17 inhibitory activity as described in example 22.

In some embodiments, the amino acid sequence of AAT, the variant, isoform or fragment thereof, as described herein, is at least 80% identical to the corresponding amino acid sequence in SEQ ID NO: 1. In some embodiments, the amino acid sequence of AAT, the variant, isoform or fragment thereof is 80%, 81%, 82%, 83%, 84%, 85%, 86%, 87%, 88%, 89%, 90%, 91%, 92%, 93%, 94%, 95%, 96%, 97%, 98%, or 99% identical to a corresponding amino acid sequence in SEQ ID NO: 1.

The terms “nucleic acid”, “polynucleotide”, and “oligonucleotide” are used interchangeably and refer to any kind of deoxyribonucleotide (e.g. DNA, cDNA, . . . ) or ribonucleotide (e.g. RNA, mRNA, . . . ) polymer or a combination of deoxyribonucleotide and ribonucleotide (e.g. DNA/RNA) polymer, in linear or circular conformation, and in either single- or double-stranded form. These terms are not to be construed as limiting with respect to the length of a polymer and can encompass known analogues of natural nucleotides, as well as nucleotides that are modified in the base, sugar and/or phosphate moieties (e.g. phosphorothioate backbones). In general, an analogue of a particular nucleotide has the same base-pairing specificity; i.e., an analogue of A will base-pair with T.

The term “treatment” (and grammatical variations thereof such as “treat” or “treating”), as used herein, refers to clinical intervention in an attempt to alter the natural course of the individual being treated, and can be performed either for prophylaxis or during the course of clinical pathology. Desirable effects of treatment include, but are not limited to, preventing occurrence or recurrence of disease, alleviation of symptoms, diminishment of any direct or indirect pathological consequences of the disease, decreasing the rate of disease progression, amelioration or palliation of the disease state, and remission or improved prognosis. In some embodiments, antibodies of the invention are used to delay development of a disease or to slow the progression of a disease.

CIDP is an acquired polyneuropathy within the peripheral nerve system with an assumed autoimmune-mediated pathogenesis. CIDP is characterized by symmetrical weakness in both proximal and distal muscles that worsens progressively. The condition is usually, but not always, associated with impaired sensation, absent or diminished tendon reflexes, an elevated cerebrospinal fluid protein level, and changes in electrophysiology parameters. Nerve biopsy specimens are characterized by signs of demyelination. The clinical course can be relapsing or chronic and progressive (see, e.g., Mathey E K, et al. J Neurol Neurosurg Psychiatry 2015; 86:973-985; Koller H, et al. N Engl J Med. 2005; 352(13): 1343-1356), the former being much more common in young adults. CIDP is a rare disease with an estimated prevalence of about 1.6 to 8.9 per 100,000 adults and about 0.5 per 100,000 children. CIDP may be diagnosed as described by the Joint Task Force of the EFNS and the PNS (Journal of the Peripheral Nervous System 15:1-9 (2010)).

The following conditions are identical or considered essentially identical to CIDP and are thus encompassed by the term “CIDP”: “chronic relapsing polyneuropathy”, “chronic idiopathic demyelinating polyneuropathy”, “chronic inflammatory demyelinating polyradiculoneuropathy”, and “chronic acquired demyelinating polyneuropathy” (“CADP”).

The inventors found that AAT can reduce neuronal pathology pathways (FIG. 2A-4C, Table 1-10). This reduction of neuronal pathology pathways was observed in resting cells (FIG. 4B) and stimulated cells (FIG. 4C) and is therefore useful in preventing and/or treating diseases or disorders of the nervous system and symptoms thereof.

TACE/ADAM17 activity modulation is involved in myelin regulation and as an inflammation hallmark of chronic inflammatory demyelinating polyneuropathy.

The inventors found that AAT is able to inhibit TACE/ADAM17 in a dose-dependent manner which, without being bound by theory, rescues myelin production by SCs, and thus subsequently prevents, or slowing, and/or reverses the progression of chronic inflammatory demyelinating polyneuropathy. As such, the combined anti-inflammatory and neuroprotective/regenerative effect is surprisingly effective in the treatment of chronic inflammatory demyelinating polyneuropathy.

Accordingly, the invention is at least in part based on the finding that AAT is useful in treating disease or disorders of the nervous system as described herein.

According to some embodiments, the treatment described herein comprises single dose administration of the total amount of AAT. According to certain embodiments, the effective amount of AAT is about 10 mg to about 1000 mg per kg body weight of AAT per week, preferably about 10 mg to about 500 mg per kg body weight of AAT per week (or an equivalent amount of AAT variant, fragment, isoform and/or corresponding nucleic acid). According to certain embodiments, the effective amount of AAT is about 30 mg to about 240 mg per kg body weight AAT per week, preferably about 60 mg to about 120 mg per kg body weight AAT per week (or an equivalent amount of AAT variant, fragment, isoform and/or corresponding nucleic acid). According to certain embodiments, the effective amount of AAT (or an equivalent amount of AAT variant, fragment, isoform and/or corresponding nucleic acid) is about 10 mg/kg/week to about 1000 mg/kg/week, preferably about 10 mg/kg/week to about 500 mg/kg/week. According to certain embodiments, the effective amount of AAT (or an equivalent amount of AAT variant, fragment, isoform and/or corresponding nucleic acid) is above about 100 mg/kg/day, preferably between about 100 mg/kg/day to about 1000 mg/kg/day, more preferably between about 100 mg/kg/day to about 500 mg/kg/day, more preferably between about 150 mg/kg/day to about 250 mg/kg/day, more preferably between about 150 mg/kg/day to about 210 mg/kg/day.

According to other embodiments, the treatment described herein comprises multiple administrations of multiple portion doses to reach the total cumulative dose of AAT. According to certain embodiments, each portion dose comprises from about 5 mg to about 500 mg per kg, preferably about 15 mg to about 240 mg per kg. According to other embodiments, each portion dose comprises about 10, about 20, about 40, about 60, about 80, about 120 or about 240 mg AAT/kg (or an equivalent amount of AAT variant, fragment, isoform and/or corresponding nucleic acid or small molecule). Each possibility represents a separate embodiment of the present invention.

In one embodiment, the invention relates to a kit of parts for use in the treatment of a disease or disorder of the nervous system, the kit comprising: i) a) the alpha1-antitrypsin (AAT) protein, variant, isoform and/or a fragment thereof, wherein said variant, isoform and/or fragment has inhibitory activity; and/or b) the nucleic acid encoding AAT, variant, isoform and/or fragment thereof having ADAM17 inhibitory activity and ii) a plurality of IgG antibodies, isoforms thereof, fragments thereof, and/or IgG variants.

The term “plurality of IgG antibodies”, as used herein, refers to at least two different, at least three different, at least four different, at least five different, at least six different, at least seven different, at least eight different, at least nine different or at least ten different IgG antibodies. In certain embodiments, the plurality of IgG antibodies identify at least two different epitopes or antigens. In certain embodiments, the plurality of IgG antibodies comprises IgG antibodies of several IgG subclasses. In certain embodiments, the plurality of IgG antibodies are polyclonal antibodies. In certain embodiments, the plurality of IgG antibodies are not monoclonal antibodies. In certain embodiments, the plurality of IgG antibodies described herein comprises IgG antibodies human plasma derived IgG antibodies, preferably pooled IgG antibodies. Isoforms of IgG antibodies, fragments of IgG antibodies and/or IgG variants may further be combined with (e.g. added to or not separated from) the plurality of IgG antibodies. In certain embodiments, these isoforms, fragments and variants are naturally occurring isoforms, fragments and/or variants. In other embodiments, the isoforms, fragments and/or variants are separately produced and added to the IgG antibodies.

In general, the term “antibody” is used herein in the broadest sense and encompasses various antibody structures, including but not limited to monoclonal antibodies, polyclonal antibodies, multispecific antibodies (e.g., bispecific antibodies), fully-human antibodies and antibody fragments so long as they exhibit the desired antigen-binding activity. In some embodiments, the IgG antibodies described herein are from the IgG1, IgG2, IgG3, and/or IgG4 subclass. In some embodiments, the IgG antibodies described herein comprise polyclonal IgG antibodies. In some embodiments, the IgG antibodies described herein are part of an immunoglobulin therapy. As used herein, the term “immunoglobulin therapy” refers generally to a therapeutic method of intravenously, subcutaneously, or intramuscularly administering a composition of IgG immunoglobulins to a patient for treating a number of conditions such as immune deficiencies, inflammatory diseases, and autoimmune diseases. The IgG immunoglobulins are typically pooled and prepared from plasma. Whole antibodies or fragments can be used. IgG immunoglobulins can be formulated in higher concentrations (e.g., greater than 10%) for subcutaneous administration, or formulated for intramuscular administration. This is particularly common for specialty IgG preparations which are prepared with higher than average titres for specific antigens (e.g., Rho D factor, pertussis toxin, tetanus toxin, botulism toxin, rabies, etc.). Preparation of an immunoglobulin therapy is known in the art (see e.g. U.S. Pat. No. 8,940,877B2). The immunoglobulin therapy may be derived from mammalian, preferably human, plasma. In certain embodiments, the plasma of multiple (generally 1000 or more) healthy donors is pooled and optionally further processed. The term “healthy individual” means an individual who meets the current (at the time of donation) standard eligibility criteria for donating blood, bearing in mind that such eligibility criteria are subject to continuous improvement and change. In some embodiments, the immunoglobulin fraction is enriched from the pooled plasma. Preferably, the immunoglobulin is purified from the pooled plasma. More preferably, the immunoglobulin is purified and concentrated. In various embodiments, purified and concentrated immunoglobulin G (IgG) is used. In certain embodiments, the immunoglobulin therapy may contain traces of immunoglobulins of different Ig classes such as IgA or IgM. In certain embodiments, the IgA concentration is 50 μg or less per 100 mg immunoglobulin. In a preferred embodiment, the IgA concentration is 25 μg or less per 100 mg immunoglobulin. Low IgA is desirable in order to avoid adverse events in patients with IgA deficiency. In one embodiment, the IgM concentration is 10 μg or less per 100 mg immunoglobulin. In a preferred embodiment, the IgM concentration is 5 μg or less per 100 mg immunoglobulin. In various embodiments, the immunoglobulin therapy exhibits a purity of the protein fraction of >90% IgG, more preferably >95% IgG, even more preferably >98% IgG. In various embodiments, the immunoglobulin therapy exhibits an immunoglobulin monomer and dimer content of >90%, more preferably >95%, even more preferably >98%. The immunoglobulin therapy preferably exhibits a natural IgG subclass distribution. In one embodiment, the immunoglobulin subclass distribution in the immunoglobulin therapy is 62-74% IgG1, 22-34% IgG2, 2-5% IgG3 and 1-3% IgG4. The immunoglobulin therapy may contain additional ingredients such as stabilizers, for example amino acids such as proline or glycine, or sucrose, maltose, sorbitol, albumin nicotinamide, PEG, polysorbate 80, or others. Preferred stabilizers are amino acids, in particular proline. In various embodiments, the immunoglobulin therapy contains 10-30% (w/v) immunoglobulin. In certain embodiments, the immunoglobulin therapy is provided as a solution containing at least 10% (w/v) immunoglobulin, more preferably at least 15% (w/v) immunoglobulin, most preferably about 20% (w/v) immunoglobulin. The immunoglobulin therapy may also contain about 30% (w/v) immunoglobulin. The immunoglobulin therapy is virus-safe for enveloped viruses (e.g., HIV, HBV and HCV) and non-enveloped viruses (e.g., HAV and parovirus B19). The immunoglobulin therapy may be provided as a liquid product or a lyophilized product. In a preferred embodiment, the immunoglobulin therapy is provided as a liquid product. Such liquid products are ready-for-use, i.e., it is not necessary to reconstitute the product prior to administration. Liquid products are convenient to use, as no reconstitution is required. Therefore, liquid products are particularly suitable for self-administration by patients. In some embodiments, the dose of IgG antibodies is individually adjusted or a fixed dose. In some embodiments, the IgG antibodies described herein are administered a dose selected from the range of about 0.1-about 0.4 g/kg patient weight, from the range of about 0.1-about 0.3 g/kg patient weight, from the range of about 0.15-about 0.25 g/kg patient weight, from the range of about 0.18-about 0.22 g/kg patient weight or a dose of about 0.2 g/kg patient weight per 5-10 days or per 6-8 days per week, preferably in a dose selected from the range of about 0.18-about 0.22 g/kg patient weight per 6-8 days. In some embodiments, the dose of IgG antibodies is administered in a single dose or distributed 2, 3, 4, 5, 6 or 7 times a week.

The term “disease or disorder of the nervous system”, as used herein, refers to a group of disease or disorders, wherein the pathology involves the nervous system. In some embodiments, the disease or disorder of the nervous system described herein is a disease or disorder selected from the group consisting of 12q14 microdeletion syndrome, 15q13.3 microdeletion syndrome, 15q24 microdeletion syndrome, 22q11.2 deletion syndrome, 22q13.3 deletion syndrome, 2-methylbutyryl-CoA dehydrogenase deficiency, 2q23.1 microdeletion syndrome, 2q37 deletion syndrome, 3-alpha hydroxyacyl-CoA dehydrogenase deficiency, 3MC syndrome, XXXY syndrome, XYYY syndrome, XXXXY syndrome, 5q14.3 microdeletion syndrome, 6-pyruvoyl-tetrahydropterin synthase deficiency, Aarskog syndrome, Abetalipoproteinemia, ABri amyloidosis, absence of septum pellucidum, Aceruloplasminemia, Acrocallosal syndrome, acrofacial dysostosis Catania type, acrofacial dysostosis Rodriguez type, acute cerebellar ataxia, acute cholinergic dysautonomia, acute CNS demyelinating event, acute disseminated encephalomyelitis, acute inflammatory demyelinating polyneuropathy, acute intermittent porphyria, acute motor and sensory axonal neuropathy syndrome, ADCY5-related dyskinesia, adenosine monophosphate deaminase 1 deficiency, adenylosuccinase deficiency, Adie syndrome, adrenomyeloneuropathy, adult polyglucosan body disease, adult-onset nemaline myopathy, advanced sleep phase syndrome, agenesis of the corpus callosum, age-related peripheral neuropathy, age-related peripheral neuropathy, Agnosia, Aicardi syndrome, Aicardi-Goutieres syndrome, AIDS Dementia Complex, Al Gazali Aziz Salem syndrome, Alaninuria, Albinism deafness syndrome, alcohol or nutritional deficiencies induced sensorimotor deficiency, alcoholic neuropathy, alcoholic peripheral neuropathy, Alexander disease, ALG 11-CDG (CDG-Ip), ALG12-CDG (CDG-Ig), ALG13-CDG, ALG1-CDG (CDG-Ik), ALG2-CDG (CDG-Ii), ALG3-CDG (CDG-Id), ALG6-CDG (CDG-Ic), ALG8-CDG (CDG-Ih), ALG9-CDG (CDG-IL), Allan-Herndon-Dudley syndrome, alopecia epilepsy oligophrenia syndrome of Moynahan, alopecia, epilepsy, pyorrhea, mental subnormality, alopecia-contractures-dwarfism-intellectual disability syndrome, alopecia-intellectual disability syndrome, Alpers syndrome, alpha-ketoglutarate dehydrogenase deficiency, alpha-mannosidosis, alpha-thalassemia x-linked intellectual disability syndrome, alternating hemiplegia of childhood, Alzheimer disease type 4, Alzheimer's disease, Alzheimer's disease without neurofibrillary tangles, aminoacylase 1 deficiency, aminolevulinate dehydratase deficiency porphyria, Amish lethal microcephaly, Amish Nemaline Myopathy, amyloid neuropathy, amyopathic dermatomyositis, amyotrophic lateral sclerosis, amyotrophic lateral sclerosis type 6, amyotrophic lateral sclerosis-parkinsonism/dementia complex 1, amytrophic lateral sclerosis, anaplastic astrocytoma, anaplastic ganglioglioma, anaplastic oligodendroglioma, Andermann syndrome, Andersen-Tawil syndrome, anemia sideroblastic ataxia, spinocerebellar ataxia, Anencephaly, Angioma hereditary neurocutaneous, Aniridia, Aniridia renal agenesis psychomotor retardation, Antisynthetase syndrome, Aortic arch anomaly, Apraxia, Arachnoid cysts, Arachnoiditis, Aromatic L-amino acid decarboxylase deficiency, Arthrogryposis multiplex congenita, distal, X-linked, Arthrogryposis renal dysfunction cholestasis syndrome, Arts syndrome, Aspartylglycosaminuria, Ataxia, Ataxia telangiectasia, Ataxia with oculomotor apraxia type 1, Ataxia with Oculomotor Apraxia Type 2, Ataxia with oculomotor apraxia type 4, Ataxia with vitamin E deficiency, Ataxia-teleangiectasia, Atelosteogenesis type 2, Atelosteogenesis type 3, Atkin syndrome, Atypical Rett syndrome, aseptic meningitis, Autism with port-wine stain, Autosomal dominant centronuclear myopathy, Autosomal dominant cerebellar ataxia/deafness/narcolepsy, autosomal dominant Charcot-Marie-Tooth disease type 2 with giant axons, autosomal dominant deafness-onychodystrophy syndrome, autosomal dominant intermediate Charcot-Marie-Tooth disease, autosomal dominant leukodystrophy with autonomic disease, autosomal dominant neuronal ceroid lipofuscinosis 4B, autosomal dominant nocturnal frontal lobe epilepsy, autosomal dominant non-syndromic intellectual disability, autosomal dominant optic atrophy plus syndrome, autosomal dominant partial epilepsy with auditory features, autosomal dominant spinal muscular atrophy, autosomal recessive axonal neuropathy with neuromyotonia, autosomal recessive centronuclear myopathy, autosomal recessive Charcot-Marie-Tooth disease with hoarseness, autosomal recessive intermediate Charcot-Marie-Tooth disease type A, autosomal recessive intermediate Charcot-Marie-Tooth disease type B, autosomal recessive juvenile Parkinson disease, Autosomal recessive neuronal ceroid lipofuscinosis 4A, Adult neuronal ceroid lipofuscinosis, Autosomal recessive primary microcephaly, Autosomal recessive spastic ataxia 4, Autosomal recessive spastic paraplegia type 49, Autosomal recessive spinocerebellar ataxia 9, B4GALT1-CDG (CDG-IId), Bannayan-Riley-Ruvalcaba syndrome, Barth syndrome, Battaglia-Neri syndrome, Becker muscular dystrophy, Behavioral variant of frontotemporal dementia, Behget disease, Bell's palsy, Benign essential blepharospasm, Benign familial neonatal epilepsy, Benign familial neonatal-infantile seizures, Benign hereditary chorea, Benign rolandic epilepsy (BRE), Beta-Propeller Protein-Associated Neurodegeneration, Bethlem myopathy, bilateral frontal polymicrogyria, bilateral frontoparietal polymicrogyria, bilateral generalized polymicrogyria, bilateral parasagittal parieto-occipital polymicrogyria, bilateral perisylvian polymicrogyria, Binswanger's disease, Biotinidase deficiency, Biotin-thiamine-responsive basal ganglia disease, Birk-Barel syndrome, Bixler Christian Gorlin syndrome, Blepharonasofacial malformation syndrome, Bobble-head doll syndrome, Bohring-Opitz syndrome, Borjeson-Forssman-Lehmann syndrome, Bowen-Conradi syndrome, Brachioskeletogenital syndrome, Brachydactyly-mesomelia-intellectual disability-heart defects syndrome, Brain dopamine-serotonin vesicular transport disease, Brain-lung-thyroid syndrome, Branchial arch syndrome X-linked, Brody myopathy, Brooks Wisniewski Brown syndrome, Brown-Sequard syndrome, Bullous dystrophy, C syndrome, Cabezas syndrome, CADASIL, Camptocormism, Camptodactyly arthropathy coxa vara pericarditis syndrome, CANOMAD syndrome, Cantu syndrome, Cap myopathy, Cardiofaciocutaneous syndrome, Carey-Fineman-Ziter syndrome, Carney complex, Cataract ataxia deafness, Catel Manzke syndrome, Caudal appendage deafness, Caudal regression sequence, Central core disease, Central nervous system germinoma, Central neurocytoma, Central pain syndrome, Central pontine myelinolysis, Cerebellar ataxia, Cerebellar degeneration, Cerebellar hypoplasia, Cerebellitis, Cerebelloparenchymal disorder 3, Cerebellum agenesis hydrocephaly, Cerebral autosomal recessive arteriopathy, Cerebral cavernous malformation, Cerebral dysgenesis, neuropathy, ichthyosis, and palmoplantar keratoderma syndrome, Cerebral folate deficiency, Cerebral gigantism jaw cysts, Cerebral palsy, Cerebral palsy ataxic, Cerebral palsy athetoid, Cerebral palsy spastic hemiplegic, Cerebral palsy spastic monoplegic, Cerebral palsy spastic quadriplegic, Cerebral sclerosis, Cerebro-facio-articular syndrome, Cerebro-oculo-facio-skeletal syndrome, Cerebrooculonasal syndrome, Cerebrospinal fluid leak, Cerebrotendinous xanthomatosis, Ceroid lipofuscinosis neuronal 1, Cervical hypertrichosis peripheral neuropathy, Chanarin-Dorfman syndrome, Charcot-Marie-Tooth disease, Charcot-Marie-Tooth disease type 1A, Charcot-Marie-Tooth disease type 2, Charcot-Marie-Tooth disease type 3, Charcot-Marie-Tooth disease type 4, Chediak-Higashi syndrome, Chiari malformation, Chiari malformation type 1, Chiari malformation type 2, Chiari malformation type 4, Childhood apraxia of speech, Childhood-onset nemaline myopathy, Chorea-acanthocytosis, Choroid plexus carcinoma, Choroid plexus papilloma, Christianson syndrome, Chromosome 17p13.1 deletion syndrome, Chromosome 1711.2 deletion syndrome, Chromosome 19q13.11 deletion syndrome, Chromosome lp36 deletion syndrome, Chromosome 3p-syndrome, Chronic hiccups, Chronic lymphocytic inflammation, Chronic progressive external ophthalmoplegia, Chudley Rozdilsky syndrome, Cisplatin induced sensory neuropathy, Cleft palate short stature vertebral anomalies, Cluster headache, COACH syndrome, COASY protein-associated neurodegeneration, coats disease, Cobb syndrome, Cockayne syndrome type I, Cockayne syndrome type II, Cockayne syndrome type III, Coenzyme Q10 deficiency, Coffin-Lowry syndrome, Coffin-Siris syndrome, COGI-CDG (CDG-IIg), COG4-CDG (CDG-IIj), COG5-CDG (CDG-IIi), COG7-CDG (CDG-IIe), COG8-CDG (CDG-IIh), Cohen syndrome, cold-induced sweating syndrome, complex regional pain syndrome, congenital central hypoventilation syndrome, congenital cytomegalovirus, congenital fiber type disproportion, congenital fibrosis of extraocular muscles, congenital generalized lipodystrophy type 4, congenital insensitivity to pain, congenital insensitivity to pain with anhidrosis, congenital intrauterine infection-like syndrome, congenital laryngeal palsy, congenital mirror movement disorder, congenital muscular dystrophy, congenital myasthenic syndrome, congenital rubella, congenital toxoplasmosis, continuous spike-wave during slow sleep syndrome, convulsions, corneal hypesthesia, Cornelia de Lange syndrome, Corpus callosum agenesis, Cortical blindness, Cortical dysgenesis, Corticobasal degeneration, Costello syndrome, Crane-Heise syndrome, cranial nerve palsy, Craniofrontonasal dysplasia, Craniopharyngioma, Craniorachischisis, Craniotelencephalic dysplasia, Creutzfeldt-Jakob disease, Crome syndrome, Curry Jones syndrome, cylindrical spirals myopathy, cyprus facial neuromusculoskeletal syndrome, cytomegalic inclusion disease, D-2-hydroxyglutaric aciduria, Dandy-Walker cyst, Dandy-Walker like malformation, Dandy-Walker malformation, Danon disease, Dapsone induced neuropathy, DDOST-CDG (CDG-Ir), DEAF1-associated disorders, Dentatorubral-pallidoluysian atrophy, Dermatomyositis, Developmental dysphasia familial, Diabetic neuropathy, Dihydrolipoamide dehydrogenase deficiency, Dihydropteridine reductase deficiency, Diphtheria, Distal myopathy with vocal cord weakness, DOOR syndrome, Dopamine beta hydroxylase deficiency, Dopamine transporter deficiency syndrome, Dopa-responsive dystonia, DPAGTT-CDG (CDG-Ij), DPMT-CDG (CDG-Ie), DPM2-CDG, DPM3-CDG (CDG-Io), Dravet syndrome, Duane syndrome, Dubowitz syndrome, Duchenne muscular dystrophy, Dykes Markes Harper syndrome, Dysautonomia like disorder, Dysequilibrium syndrome, Dyskeratosis congenita, Dyskeratosis congenita autosomal dominant, Dyskeratosis congenita autosomal recessive, Dyskeratosis congenita X-linked, Dyssynergia cerebellaris myoclonica, Dystonia 2, DYT-PRKRA, DYT-THAP1, DYT-TOR1A, DYT-TUBB4A, early infantile epileptic encephalopathy, early infantile epileptic encephalopathy 25, early-onset anterior polar cataract, early-onset autosomal dominant alzheimer disease, early-onset parkinsonism-intellectual disability syndrome, eastern equine encephalitis, empty sella syndrome, encephalitis lethargica, encephalocraniocutaneous lipomatosis, encephalopathy, eosinophilic fasciitis, eosinophilic granulomatosis, ependymoma, epidermolysa bullosa simplex with muscular dystrophy, epilepsy juvenile absence, epilepsy occipital calcifications, epilepsy progressive myoclonic type 3, epilepsy with myoclonic-atonic seizures, epiphyseal dysplasia hearing loss dysmorphism, episodic ataxia, erythromelalgia, essential tremor, Fabry disease, facial onset neuronopathy, facioscapulohumeral muscular dystrophy, Fallot complex, familial amyloidosis, familial bilateral striatal necrosis, familial caudal dysgenesis, familial congenital palsy of trochlear nerve, familial dysautonomia, familial encephalopathy, familial exudative vitreoretinopathy, familial focal epilepsy, familial hemiplegic migraine, familial hemophagocytic lymphohistiocytosis, familial infantile convulsions familial infantile paroxysmal choreoathetosis, familial porencephaly, familial transthyretin amyloidosis, familiar or sporadic hemiplegic migraine, farber disease, fatal familial insomnia, fatal infantile encephalomyopathy, fatty acid hydroxylase-associated neurodegeneration, FBXL4-related encephalomyopathic mitochondrial DNA depletion syndrome, Febrile infection-related epilepsy syndrome, Feigenbaum Bergeron Richardson syndrome, Filippi syndrome, Fine-Lubinsky syndrome, Fingerprint body myopathy, Fitzsimmons Walson Mellor syndrome, Fitzsimmons-Guilbert syndrome, Floating-Harbor syndrome, Flynn Aird syndrome, focal dermal hypoplasia, focal motor weakness, focal segmental glomerulosclerosis, Fountain syndrome, FOXG1 syndrome, Fragile X syndrome, Fragile XE syndrome, Friedreich ataxia, Frontometaphyseal dysplasia, Frontotemporal dementia, Frontotemporal lobar dementia, Fryns syndrome, Fucosidosis, Fukuyama type muscular dystrophy, Fumarase deficiency, Galactosialidosis, Galloway-Mowat syndrome, Gamma aminobutyric acid transaminase deficiency, Gangliocytoma, GAPO syndrome, Gaucher disease type 1, Gaucher disease type 2, Gaucher disease type 3, Gemignani syndrome, Genitopatellar syndrome, Genoa syndrome, Gerstmann syndrome, Gerstmann-Straussler-Scheinker disease, Giant axonal neuropathy, Gillespie syndrome, Gliomatosis cerebri, Glucose transporter type 1 deficiency syndrome, Glutamine deficiency, congenital, Glutaric acidemia type I, Glutaric acidemia type II, Glutaric acidemia type III, Glycogen storage disease type 13, Glycogen storage disease type 2, Glycogen storage disease type 3, Glycogen storage disease type 4, Glycogen storage disease type 5, Glycogen storage disease type 7, GM1 gangliosidosis type 1, GM1 gangliosidosis type 2, GM1 gangliosidosis type 3, GM3 synthase deficiency, GMS syndrome, Goldberg-Shprintzen megacolon syndrome, Gomez Lopez Hemandez syndrome, GOSR2-related progressive myoclonus ataxia, Graham-Cox syndrome, Granulomatosis with polyangiitis, Griscelli syndrome type 1, Grubben de Cock Borghgraef syndrome, GTP cyclohydrolase I deficiency, GTPCH1-deficient DRD, Guanidinoacetate methyltransferase deficiency, Guillain-Barre syndrome, Gurrieri syndrome, Gyrate atrophy of choroid and retina, Hair defect-photosensitivity-intellectual disability syndrome, Hallermann-Streiff syndrome, Hall-Riggs syndrome, Hamanishi Ueba Tsuji syndrome, Hansen's disease, Harding ataxia, Harlequin syndrome, Harrod Doman Keele syndrome, Hartnup disease, Hashimoto encephalopathy, Hemangioblastoma, Hemicrania continua, Hemimegalencephaly, Hennekam syndrome, hereditary angiopathy, hereditary coproporphyria, hereditary diffuse leukoencephalopathy, hereditary fibrosing poikiloderma with tendon contractures, myopathy, and pulmonary fibrosis, hereditary geniospasm, hereditary hemorrhagic telangiectasia, hereditary hemorrhagic telangiectasia type 2, hereditary hemorrhagic telangiectasia type 3, hereditary hemorrhagic telangiectasia type 4, hereditary hyperekplexia, hereditary motor and sensory neuropathy type 5, hereditary neuropathy with liability to pressure palsies (HNPP), hereditary predisposition to pressure palsies (focal and symmetrical), hereditary proximal myopathy with early respiratory failure, hereditary sensorimotor neuropathy with hyperelastic skin, hereditary sensory and autonomic neuropathy type 1e, hereditary sensory and autonomic neuropathy type 2, hereditary sensory and autonomic neuropathy type 1-7 (HSAN I-VII), hereditary sensory and autonomic neuropathy type v, hereditary sensory neuropathy type 1, hereditary spastic paraplegia, hereditary vascular retinopathy, Hernindez-Aguirre Negrete syndrome, herpes simplex encephalitis, herpes zoster oticus, HIBCH deficiency, Homocystinuria, Horizontal gaze palsy with progressive scoliosis, Hoyeraal Hreidarsson syndrome, HSD10 disease, HTLV-1 associated myelopathy/tropical spastic paraparesis, Human HOXA1 Syndromes, Human immunodeficiency virus induced neuropathy, Huntington disease, Huntington's disease, Hurler syndrome, Hurler-Scheie syndrome, hydranencephaly, hydrocephalus (e.g. due to congenital stenosis of aqueduct of sylvius), hydrocephalus-cleft palate-joint contractures syndrome, hydroxykynureninuria, hyperbetaalaninemia, hypercoagulability syndrome due to glycosylphosphatidylinositol deficiency, hyperkalemic periodic paralysis, hypermethioninemia, hyperphenylalaninemia, hyperprolinemia, hyperprolinemia type 2, hypertrophic neuropathy of Dejerine-Sottas, hypocalcemia, autosomal dominant, hypokalemic periodic paralysis, hypomelanosis of Ito, hypomyelination (e.g., with atrophy of basal ganglia and/or cerebellum), hypoparathyroidism-intellectual disability-dysmorphism syndrome, hypospadias-intellectual disability, Goldblatt type syndrome, hypothalamic hamartomas, ichthyosis alopecia eclabion ectropion intellectual disability, idiopathic intracranial hypertension, idiopathic spinal cord herniation, inclusion body myositis, incontinentia pigmenti, infantile axonal neuropathy, infantile cerebellar retinal degeneration, infantile choroidocerebral calcification syndrome, infantile myofibromatosis, infantile neuroaxonal dystrophy, infantile onset spinocerebellar ataxia, infantile spasms broad thumbs, infantile-onset ascending hereditary spastic paralysis, infection-induced acute encephalopathy 3, intellectual deficit Buenos-Aires type, athetosis intellectual disability, hypoplastic corpus callosum intellectual disability, intellectual disability-developmental delay-contractures syndrome, intellectual disability-dysmorphism-hypogonadism-diabetes mellitus syndrome, Intellectual disability-severe speech delay-mild dysmorphism syndrome, intellectual disability-spasticity-ectrodactyly syndrome, Intermediate congenital nemaline myopathy, Internal carotid agenesis, Intraneural perineurioma, IRVAN syndrome, Isaacs' syndrome, Isodicentric chromosome 15 syndrome, Johanson-Blizzard syndrome, Johnson neuroectodermal syndrome, Joubert syndrome, Juberg Marsidi syndrome, Juvenile amyotrophic lateral sclerosis, Juvenile dermatomyositis, Juvenile Huntington disease, Juvenile polymyositis, Juvenile primary lateral sclerosis, Kabuki syndrome, Kanzaki disease, Kapur Toriello syndrome, Kaufman oculocerebrofacial syndrome, KBG syndrome, KCNQ2-Related Disorders, Keams-Sayre syndrome, Kennedy disease, Keratosis follicularis dwarfism, cerebral atrophy, Kernicterus, Keutel syndrome, King Denborough syndrome, Kleefstra syndrome, Kleine Levin syndrome, Klumpke paralysis, Kosztolanyi syndrome, Kozlowski-Krajewska syndrome, Krabbe's disease, Kuru, Kuzniecky Andermann syndrome, L-2-hydroxyglutaric aciduria, La Crosse encephalitis, Laband syndrome, Lafora disease, Laing distal myopathy, Lambert Eaton myasthenic syndrome, Landau-Kleffner syndrome, l-arginine:glycine amidinotransferase deficiency, Late-onset distal myopathy, Markesbery-Griggs type, lateral meningocele syndrome, Laurence-Moon syndrome, LCHAD deficiency, Leber hereditary optic neuropathy, Leigh syndrome, Lennox-Gastaut syndrome, Lenz Majewski hyperostotic dwarfism, Lenz microphthalmia syndrome, Lesch Nyhan syndrome, Leukodystrophy, Leukoencephalopathy (e.g. with thalamus and brainstem involvement and high lactate), Levic Stefanovic Nikolic syndrome, Lewis-Sumner syndrome, Lhermitte-Duclos disease, Li-Fraumeni syndrome, limb-girdle muscular dystrophy (e.g. type 1A, 1B, 1C, 1D, 1E, 1F, 1G, 1H, 2A, 2B, 2C, 2D, 2E, 2F, 2H, 2I, 2J, 2K, 2L, 2M, 2N, 2O, 2P, 2Q, 2S, 2T), limbic encephalitis with LGI1 antibodies, Limited cutaneous systemic sclerosis, Lipoic acid synthetase deficiency, Lissencephaly 1, Lissencephaly 2, Lissencephaly X-linked, Localized hypertrophic neuropathy, Locked-in syndrome, Logopenic progressive aphasia, Lowe oculocerebrorenal syndrome, Lowry Maclean syndrome, Lujan syndrome, Lyme disease, Mac Dermot Winter syndrome, macrocephaly-short stature-paraplegia syndrome, macrothrombocytopenia progressive deafness, mal de debarquement syndrome, male pseudohermaphroditism intellectual disability syndrome, malignant hyperthermia, malignant hyperthermia arthrogryposis torticollis, malignant migrating partial seizures of infancy, MAN1B1-CDG, Mandibulofacial dysostosis (e.g. with microcephaly), Mannosidosis, Marchiafava Bignami disease, Marden-Walker syndrome, Marfanoid habitus-autosomal recessive intellectual disability syndrome, Marinesco-Sjogren syndrome, Martsolf syndrome, McDonough syndrome, McLeod neuroacanthocytosis syndrome, Meckel syndrome, MECP2 duplication syndrome, medrano roldan syndrome, medulloblastoma, megalencephalic leukoencephalopathy(e.g. with subcortical cysts), megalencephaly-polymicrogyria-polydactyly-hydrocephalus syndrome, megaloblastic anemia, megalocomea-intellectual disability syndrome, Mehes syndrome, MEHMO syndrome, Meier-Gorlin syndrome, Meige syndrome, Melnick-Needles syndrome, Meningioma, meningitis, menkes disease, meralgia paresthetica, metaphyseal dysostosis-intellectual disability-conductive deafness syndrome, methionine adenosyltransferase deficiency, methylcobalamin deficiency cbl g type, methylmalonic acidemia with homocystinuria type cblc, mgat2-cdg (cdg-iia), micro syndrome, microbrachycephaly ptosis cleft lip, microcephalic osteodysplastic primordial dwarfism type 1, microcephalic osteodysplastic primordial dwarfism type 2, microcephalic primordial dwarfism, (e.g., Montreal type, Toriello type), microcephaly, microcephaly autosomal dominant, microcephaly brain defect spasticity hypernatremia, microcephaly cervical spine fusion anomalies, microcephaly deafness syndrome, microcephaly glomerulonephritis marfanoid habitus, microcephaly microcomea syndrome, microcephaly-cardiomyopathy, Microduplication Xp11.22-p11.23 syndrome, microphthalmia syndromic 10, microphthalmia syndromic 4, microphthalmia syndromic 8, microphthalmia with linear skin defects syndrome, microscopic polyangiitis, migraine (e.g. with brainstem aura), mild phenylketonuria, Miller-Dieker syndrome, Miller-Fisher syndrome, minicore myopathy with external ophthalmoplegia, mitochondrial complex i deficiency, mitochondrial complex II deficiency, mitochondrial DNA depletion syndrome, encephalomyopathic form with methylmalonic aciduria, mitochondrial DNA-associated Leigh syndrome, mitochondrial encephalomyopathy lactic acidosis and stroke-like episodes, mitochondrial membrane protein-associated neurodegeneration, mitochondrial myopathy and sideroblastic anemia, mitochondrial myopathy with diabetes, mitochondrial myopathy with lactic acidosis, mitochondrial neurogastrointestinal encephalopathy syndrome, mitochondrial trifunctional protein deficiency, mixed connective tissue disease, Miyoshi myopathy, Moebius syndrome, MOGS-CDG (CDG-IIb), Mohr-Tranebjaerg syndrome, molybdenum cofactor deficiency, monoamine oxidase A deficiency, monoclonal gammopathy of undetermined significance (MGUS), Morse-Rawnsley-Sargent syndrome, Morvan's fibrillary chorea, Mousa Al din Al Nassar syndrome, Moyamoya disease, MPDU1-CDG (CDG-If), MPI-CDG (CDG-Ib), MPV17-related hepatocerebral mitochondrial DNA depletion syndrome, mucolipidosis type 4, mucopolysaccharidosis type III, mucopolysaccharidosis type IIIA, mucopolysaccharidosis type IIIB, mucopolysaccharidosis type IIIC, mucopolysaccharidosis type IIID, multifocal motor neuropathy, multiple congenital anomalies-hypotonia-seizures syndrome, multiple congenital anomalies-hypotonia-seizures syndrome type 2, multiple myeloma, multiple sulfatase deficiency, multiple system atrophy, multiple system atrophy, multisystemic smooth muscle dysfunction syndrome, muscle eye brain disease, muscular dystrophy white matter spongiosis, megaconial type muscular dystrophy, Muscular phosphorylase kinase deficiency, Musculocontractural Ehlers-Danlos syndrome, Myasthenia gravis, Myelitis, Myelocerebellar disorder, Myelomeningocele, MYH7-related scapuloperoneal myopathy, Myhre syndrome, Myoclonic epilepsy with ragged red fibers, Myoclonus cerebellar ataxia deafness, Myoclonus-dystonia, Myoglobinuria recurrent, Myopathy with extrapyramidal signs, Myosin storage myopathy, Myotonia congenita, Myotonic dystrophy type 1, Myotonic dystrophy type 2, N syndrome, Nance-Horan syndrome, Narcolepsy, NBIA/DYT/PARK-PLA2G6, Necrotizing autoimmune myopathy, Neonatal adrenoleukodystrophy, Neonatal meningitis, Neonatal progeroid syndrome, Neu Laxova syndrome, Neuroblastoma, Neurocutaneous melanosis, Neurofaciodigitorenal syndrome, Neuroferritinopathy, Neurofibromatosis type 1, Neurofibromatosis type 2, Neuroleptic malignant syndrome, Neuromyelitis optica spectrum disorder, Neuronal ceroid lipofuscinosis, Neuronal ceroid lipofuscinosis 10, Neuronal ceroid lipofuscinosis 2, Neuronal ceroid lipofuscinosis 3, Neuronal ceroid lipofuscinosis 5, Neuronal ceroid lipofuscinosis 6, Neuronal ceroid lipofuscinosis 7, Neuronal ceroid lipofuscinosis 9, Neuronal intranuclear inclusion disease, Neuropathic pain, Neuropathy ataxia retinitis pigmentosa syndrome, Neuropathy, distal hereditary motor, Jerash type, Neuropathy, hereditary motor and sensory, Okinawa type, neurologic bladder, Neuropathy, hereditary motor and sensory, Russe type, Neutral lipid storage disease with myopathy, Nevoid basal cell carcinoma syndrome, New-onset refractory status epilepticus, Nicolaides-Baraitser syndrome, Niemann-Pick disease type A, Niemann-Pick disease type B, Niemann-Pick disease type C1, Niemann-Pick disease type C2, Non-sleep wake disorder, Nondystrophic myotonia, Noonan syndrome, Norrie disease, Northern epilepsy, Oculocerebrocutaneous syndrome, Oculofaciocardiodental syndrome, Oculopharyngeal muscular dystrophy, Oculopharyngodistal myopathy, Okamoto syndrome, Olfactory neuroblastoma, Oligoastrocytoma, Oligodendroglioma, Oliver syndrome, Olivopontocerebellar atrophy, Omphalocele cleft palate syndrome lethal, OPHN1 syndrome, Opsoclonus-myoclonus syndrome, Optic atrophy 2, Optic pathway glioma, optic neuritis, Ornithine transcarbamylase deficiency, Orofaciodigital syndrome 1, Orofaciodigital syndrome 10, Orofaciodigital syndrome 2, Orofaciodigital syndrome 3, Orofaciodigital syndrome 4, Orofaciodigital syndrome 5, Orofaciodigital syndrome 6, Orthostatic intolerance due to NET deficiency, Osteopenia and sparse hair, Osteoporosis-pseudoglioma syndrome, Oto-palato-digital syndrome type 1, Oto-palato-digital syndrome type 2, Ouvrier Billson syndrome, Pachygyria-intellectual disability-epilepsy syndrome, PACS1-related syndrome, painful orbital and systemic neurofibromas-marfanoid habitus syndrome, Pallidopyramidal syndrome, Pallister W syndrome, Pallister-Killian mosaic syndrome, Pantothenate kinase-associated neurodegeneration, paralysis agitans, paralysis juvenile, paralysis of Hunt, Paramyotonia congenita, Paraneoplastic/autoimmune (anti-Hu-associated) neuropathy, Parkinson, Parkinson disease type 3, Parkinson disease type 9, Paroxysmal exertion-induced dyskinesia, Paroxysmal extreme pain disorder, Paroxysmal hemicrania, Paroxysmal kinesigenic choreoathetosis, Paroxysomal nonkinesigenic dyskinesia, Parsonage Turner syndrome, Partington syndrome, PCDH19-related female-limited epilepsy, Pediatric autoimmune neuropsychiatric disorders associated with Streptococcus infections, PEHO syndrome, Pelizaeus-Merzbacher disease, Periventricular heterotopia, Periventricular leukomalacia, Perry syndrome, Peters plus syndrome, Pfeiffer Mayer syndrome, Pfeiffer Palm Teller syndrome, Pfeiffer-type cardiocranial syndrome, PGM3-CDG, PHACE syndrome, Phosphoglycerate kinase deficiency, Phosphoglycerate mutase deficiency, Phosphoserine aminotransferase deficiency, Photosensitive epilepsy, Pitt-Hopkins syndrome, Pitt-Hopkins-like syndrome, Plasmacytoma, Pleomorphic xanthoastrocytoma, PMM2-CDG (CDG-Ia), polyneuropathy organomegaly endocrinopathy or edema M-protein and skin abnormalities syndrome (POEMS), Poliomyelitis, POLR3-Related Leukodystrophy, Polyarteritis nodosa, Polycystic lipomembranous osteodysplasia with sclerosing leukoencephalopathy, Polyneuropathy-intellectual disability-acromicria-premature menopause syndrome, Pontine tegmental cap dysplasia, Pontocerebellar hypoplasia, Pontocerebellar hypoplasia type 1, Pontocerebellar hypoplasia type 2, Pontocerebellar hypoplasia type 3, Pontocerebellar hypoplasia type 4, Pontocerebellar hypoplasia type 5, Pontocerebellar hypoplasia type 6, post-Polio syndrome, Porphyria, posterior column ataxia, posterior column ataxia with retinitis pigmentosa, postnatal progressive microcephaly, postherpetic neuralgia, postnatal seizures, and postnatal brain atrophy, Potassium aggravated myotonia, Potocki-Lupski syndrome, PPM-X syndrome, Prader-Willi habitus, Primary amebic meningoencephalitis, Primary angiitis of the central nervous system, Primary basilar impression, Primary camitine deficiency, Primary central nervous system lymphoma, Primary Familial Brain Calcification, Primary lateral sclerosis, Primary melanoma of the central nervous system, Primary orthostatic tremor, Primary progressive aphasia, Primrose syndrome, Progressive bulbar palsy, Progressive encephalomyelitis with rigidity and myoclonus, Progressive external ophthalmoplegia, autosomal recessive 1, progressive hemifacial atrophy, progressive non-fluent aphasia, Progressive Supranuclear Palsy, prolidase deficiency, Proteus syndrome, Proud syndrome, pseudoaminopterin syndrome, Pseudocholinesterase deficiency, pseudoneonatal adrenoleukodystrophy, pseudoprogeria syndrome, pseudotrisomy 13 syndrome, pseudoxanthoma elasticum, Pudendal Neuralgia, Pure autonomic failure, pyridoxal 5′-phosphate-dependent epilepsy, pyridoxine-dependent epilepsy, pyruvate dehydrogenase phosphatase deficiency, Qazi Markouizos syndrome, Radiation induced brachial plexopathy, Ramos Arroyo Clark syndrome, Ramsay Hunt syndrome I, Ramsay Hunt syndrome II, Rapid-onset dystonia-parkinsonism, Rasmussen encephalitis, Reardon Wilson Cavanagh syndrome, Reducing body myopathy, Refsum disease, Renal dysplasia-limb defects syndrome, Renier Gabreels Jasper syndrome, Restless legs syndrome, Retinal arterial macroaneurysm with supravalvular pulmonic stenosis, Retinal vasculopathy with cerebral leukodystrophy, retrobulbar neuritis, Rett syndrome, Reversible cerebral vasoconstriction syndrome, RFT1-CDG (CDG-In), Rhabdoid tumor, Rhizomelic chondrodysplasia punctata type 1, Riboflavin transporter deficiency, Richards-Rundle syndrome, Richieri Costa Da Silva syndrome, Rigid spine syndrome, Ring chromosome 10, Ring chromosome 14, Ring chromosome 20, Rippling muscle disease, RNAse T2-deficient leukoencephalopathy, Roussy Levy syndrome, RRM2B-related mitochondrial DNA depletion syndrome, Ruvalcaba syndrome, Salla disease, Sandhoff disease, Sandifer syndrome, Sarcoidosis induced neuropathy, Say Barber Miller syndrome, Say Meyer syndrome, Scapuloperoneal syndrome, SCARF syndrome, Schaaf-Yang syndrome, Scheie syndrome, Schimke immunoosseous dysplasia, Schindler disease type 1, Schinzel Giedion syndrome, Schisis association, Schizencephaly, Schwannomatosis, Schwartz Jampel syndrome, Scott Bryant Graham syndrome, Seaver Cassidy syndrome, Seckel syndrome, Semantic dementia, Sensory ataxic neuropathy, Sepiapterin reductase deficiency, Septo-optic dysplasia spectrum, SeSAME syndrome, SETBP1 disorder, severe congenital nemaline myopathy, severe intellectual disability-progressive spastic diplegia syndrome, Gustavson type severe X-linked intellectual disability, Shapiro syndrome, Short-chain acyl-CoA dehydrogenase deficiency, Shprintzen omphalocele syndrome, Shprintzen-Goldberg craniosynostosis syndrome, Sialidosis type I, Sialidosis, type II, Sickle cell anemia, Simpson-Golabi-Behmel syndrome, Single upper central incisor, Sjogren-Larsson syndrome, SLC35A1-CDG (CDG-IIf), SLC35A2-CDG, SLC35C1-CDG (CDG-IIc), Slow-channel congenital myasthenic syndrome, Smith-Fineman-Myers syndrome, Smith-Lemli-Opitz syndrome, Smith-Magenis syndrome, Sneddon syndrome, Snyder-Robinson syndrome, Sonoda syndrome, spasmodic dysphonia, spastic ataxia charlevoix-saguenay type, spastic diplegia cerebral palsy, spastic diplegia infantile type, spastic paraplegia 1, spastic paraplegia 10, spastic paraplegia 11, spastic paraplegia 12, spastic paraplegia 13, spastic paraplegia 14, spastic paraplegia 15, spastic paraplegia 16, spastic paraplegia 17, spastic paraplegia 18, spastic paraplegia 19, spastic paraplegia 2, spastic paraplegia 23, spastic paraplegia 24, spastic paraplegia 25, spastic paraplegia 26, spastic paraplegia 29, spastic paraplegia 3, spastic paraplegia 31, spastic paraplegia 32, spastic paraplegia 39, spastic paraplegia 4, spastic paraplegia 51, spastic paraplegia 5a, spastic paraplegia 6, spastic paraplegia 7, spastic paraplegia 8, spastic paraplegia 9, spastic paraplegia facial cutaneous lesions, Spastic paraplegia-epilepsy-intellectual disability syndrome, Spastic paraplegia-glaucoma-intellectual disability syndrome, Spastic tetraplegia-retinitis pigmentosa-intellectual disability syndrome, spastic tetraplegia-thin corpus callosum-progressive postnatal microcephaly syndrome, Spina bifida occulta, spinal atrophy ophthalmoplegia pyramidal syndrome, spinal meningioma, spinal muscular atrophy 1, spinal muscular atrophy type 2, spinal muscular atrophy type 3, spinal muscular atrophy-progressive myoclonic epilepsy syndrome, spinal shock, spinocerebellar ataxia, spinocerebellar ataxia 1, spinocerebellar ataxia 10, spinocerebellar ataxia 11, spinocerebellar ataxia 12, spinocerebellar ataxia 13, spinocerebellar ataxia 14, spinocerebellar ataxia 15, spinocerebellar ataxia 17, spinocerebellar ataxia 18, spinocerebellar ataxia 19 and 22, spinocerebellar ataxia 2, spinocerebellar ataxia 20, spinocerebellar ataxia 21, spinocerebellar ataxia 23, spinocerebellar ataxia 25, spinocerebellar ataxia 26, spinocerebellar ataxia 27, spinocerebellar ataxia 28, spinocerebellar ataxia 29, spinocerebellar ataxia 3, spinocerebellar ataxia 30, spinocerebellar ataxia 31, spinocerebellar ataxia 34, spinocerebellar ataxia 4, spinocerebellar ataxia 5, spinocerebellar ataxia 7, spinocerebellar ataxia 8, spinocerebellar ataxia 9, spinocerebellar ataxia autosomal recessive 3, spinocerebellar ataxia autosomal recessive 4, spinocerebellar ataxia autosomal recessive 5, spinocerebellar ataxia autosomal recessive 6, spinocerebellar ataxia autosomal recessive 7, spinocerebellar ataxia autosomal recessive 8, spinocerebellar ataxia type 6, spinocerebellar ataxia with axonal neuropathy type 1, spinocerebellar ataxia with dysmorphism, spinocerebellar ataxia x-linked type 2, spinocerebellar ataxia x-linked type 3, spinocerebellar ataxia x-linked type 4, spinocerebellar degeneration and corneal dystrophy, split hand urinary anomalies spina bifida, split spinal cord malformation, spondyloepiphyseal dysplasia congenita, SRD5A3-CDG (CDG-Iq), SSR4-CDG, STAC3 Disorder, Status epilepticus, Steinfeld syndrome, Stiff person syndrome, Stocco dos Santos syndrome, Striatonigral degeneration infantile, Sturge-Weber syndrome, subacute sclerosing panencephalitis, subcortical band heterotopia, subependymal giant cell astrocytoma, Subependymoma, Succinic semialdehyde dehydrogenase deficiency, Susac syndrome, Symmetrical thalamic calcifications, Syndromic X-linked intellectual disability 7, Tangier disease, TANGO2-Related Metabolic Encephalopathy and Arrhythmias, Tarlov cysts, Tay-Sachs disease, Tel Hashomer camptodactyly syndrome, Telfer Sugar Jaeger syndrome, Temple syndrome, Temple-Baraitser syndrome, Temporal epilepsy, Temtamy syndrome, Tethered cord syndrome, Thoracic dysplasia hydrocephalus syndrome, Thoracic outlet syndromes, Thyrotoxic periodic paralysis, TMEM165-CDG (CDG-IIk), Toriello-Carey syndrome, Tourette syndrome, Toxic neuropathies (e.g. alcoholic neuropathy, chemotherapy-induced neuropathy), Tranebjaerg Svejgaard syndrome, Transverse myelitis, Trichinosis, Trichorhinophalangeal syndrome type 2, Trigeminal neuralgia, Triosephosphate isomerase deficiency, Triple A syndrome, Troyer syndrome, Tuberous sclerosis complex, Tubular aggregate myopathy, Tumefactive multiple sclerosis, Typical congenital nemaline myopathy, Tyrosine hydroxylase deficiency, Tyrosinemia type 1, Ullrich congenital muscular dystrophy, Unverricht-Lundborg disease, Van Benthem-Driessen-Hanveld syndrome, Van Den Bosch syndrome, Variant Creutzfeldt-Jakob disease, Variegate porphyria, Vasculitis induced neuropathy, Vein of Galen aneurysm, Vici syndrome, Viljoen Kallis Voges syndrome, Vincristine induced neuropathy, Visual snow syndrome, Vitamin B6 induced neuropathy, VLCAD deficiency, Vogt-Koyanagi-Harada disease, Von Hippel-Lindau disease, Walker-Warburg syndrome, Weaver syndrome, Welander distal myopathy, Wernicke-Korsakoff syndrome, West syndrome, Whipple disease, White matter hypoplasia-corpus callosum agenesis-intellectual disability syndrome, Wiedemann Oldigs Oppermann syndrome, Williams syndrome, Wilson disease, Wilson-Turner syndrome, Wolf-Hirschhorn syndrome, Wolman disease, Woodhouse Sakati syndrome, Worster Drought syndrome, Wrinkly skin syndrome, Wyburn-Mason syndrome, Xeroderma pigmentosum, Xia-Gibbs syndrome, XK aprosencephaly, X-linked cerebral adrenoleukodystrophy, X-linked Charcot-Marie-Tooth disease type 1, X-linked Charcot-Marie-Tooth disease type 1A, X-linked Charcot-Marie-Tooth disease type 2, X-linked Charcot-Marie-Tooth disease type 3, X-linked Charcot-Marie-Tooth disease type 4, X-linked Charcot-Marie-Tooth disease type 5, X-linked Charcot-Marie-Tooth disease type 6, X-linked complicated corpus callosum agenesis, X-linked complicated spastic paraplegia type 1, X-linked creatine deficiency, X-linked dystonia-parkinsonism/Lubag, X-linked hereditary sensory and autonomic neuropathy with deafness, X-linked intellectual disability—corpus callosum agenesis—spastic quadriparesis, X-linked intellectual disability—short stature—obesity, X-linked intellectual disability, Najm type, X-linked intellectual disability, Schimke type, Siderius type X-linked intellectual disability, Turner type X-linked intellectual disability, X-linked intellectual disability-dysmorphism-cerebral atrophy syndrome, X-linked intellectual disability-plagiocephaly syndrome, X-linked lissencephaly with abnormal genitalia, X-linked myopathy with excessive autophagy, X-linked myotubular myopathy, X-linked non-specific intellectual disability, X-linked periventricular heterotopia, X-linked skeletal dysplasia-intellectual disability syndrome, Zechi Ceide syndrome, Zellweger syndrome, and ZTTK syndrome. In some embodiments, the disease or disorder of the nervous system is a psychiatric disorder. In some embodiments, the disease or disorder of the nervous system is a disease or disorder classified according to the DSM-V (American Psychiatric Association, & American Psychiatric Association, 2013, Diagnostic and statistical manual of mental disorders: DSM-5. Arlington, VA.). In some embodiments, the disease or disorder of the nervous system is a disease or disorder of the central nervous system. In some embodiments, the disease or disorder of the nervous system is an inflammatory disease or disorder of the nervous system. In some embodiments, the disease or disorder of the nervous system described herein is a disease or disorder selected from the group consisting of dementia, multiple sclerosis, amyotrophic lateral sclerosis, Alzheimer's disease, Parkinson's disease, Huntington's disease, frontotemporal lobar dementia, ataxia-teleangiectasia, multiple system atrophy, progressive supranuclear palsy, Krabbe's disease, agenesis of the corpus callosum associated with peripheral neuropathy, Duchenne muscular dystrophy, Guillain-Barre syndrome, Charcot-Marie-Tooth disease Type TA, hereditary neuropathy with liability to pressure palsies, diabetic neuropathy, toxic neuropathies, age-related peripheral neuropathy, epilepsy, sleep disorders, encephalopathy and neuropathic pain. In some embodiments, the disease or disorder of the nervous system is a neurodegenerative disease or disorder.

In some embodiments, the disease or disorder of the nervous system described herein, is a toxin and/or drug-induced neuropathy. In some embodiments, the drug-induced neuropathy described herein is induced by, partially induced by or suspected to be induced by at least one agent selected from the group consisting of chemotherapeutic agents, TNFα inhibitors, antiretroviral agents, cardiac medications, statins and antibiotics.

In some embodiments, the drug-induced neuropathy described herein is induced by, partially induced by or suspected to be induced by at least one agent selected from the group consisting of thalidomide, disulfiram, pyridoxine, colchicine, phenytoin, lithium, chloroquine, hydroxychloroquine, cisplatin, oxaliplatin, taxane, vinca alkaloids, bortezomib, suramin, misonidazole, einfliximab, etanercept, zalcitabine, didanosine, stavudine, amiodarone, perhexiline, metronidazole, dapsone, podophyllin, fluoroquinolones, isoniazid and nitrofurantoin.

In certain embodiments, the disease or disorder of the nervous system described herein is a disease or disorder of the peripheral nervous system.

In some embodiments, the toxin-induced neuropathy described herein is induced by, partially induced by or suspected to be induced by at least one agent selected from the group consisting of organic solvents, heavy metals and organophosphates.

In some embodiments, the toxin and/or drug-induced neuropathy described herein is induced by, partially induced by or suspected to be induced by alcohol and/or cigarette smoke.

In some embodiments, the toxin and/or drug-induced neuropathy described herein is characterized by at least one selected from the group of dorsal root ganglion toxicity, microtubular axon transport function abnormalities, voltage gated abnormalities, sodium channel abnormalities and demyelination.

The inventors found that AAT can reduce neuronal pathology pathways (FIG. 2A-4C, Table 1-10). This reduction of neuronal pathology pathways was observed in resting cells (FIG. 4B) and stimulated cells (FIG. 4C) and is therefore useful in preventing and/or treating diseases or disorders of the nervous system and symptoms thereof.

As such, the effect of the combination of AAT and IgG antibodies in providing immunomodulatory, anti-inflammatory, and neuroprotective/regenerative effects in diseases and disorders of the nervous system that go beyond the expectation of the person skilled in the art.

Accordingly, the invention is at least in part based on the finding that combining AAT activity with IgG antibody activity is useful in treating diseases or disorders of the nervous system as described herein.

In one embodiment, the invention relates to a pharmaceutical composition for use in treatment of a disease or disorder of the nervous system, the composition comprising: i) a) alpha1-antitrypsin (AAT) protein, a variant, an isoform and/or a fragment thereof having protease inhibitory activity; and/or b) a nucleic acid encoding AAT, a variant, an isoform and/or a fragment thereof having protease inhibitory activity; iii) a plurality of IgG antibodies, isoforms thereof, fragments thereof, and/or IgG variants; and ii) at least one pharmaceutically acceptable carrier.

In some embodiments the pharmaceutical acceptable carrier described herein is a pharmaceutically acceptable diluent or carrier.

“Pharmaceutically acceptable diluent or carrier” means a carrier or diluent that is useful in preparing a pharmaceutical composition that is generally safe, non-toxic, and desirable, and includes carriers or diluents that are acceptable for human pharmaceutical use.

Such pharmaceutically acceptable carriers can be sterile liquids, such as water and oils, including those of petroleum, animal, vegetable or synthetic origin, such as peanut oil, soybean oil, mineral oil, sesame oil and the like. Water is a preferred carrier when the pharmaceutical composition is administered intravenously. Saline solutions and aqueous dextrose and glycerol solutions can also be employed as liquid carriers, particularly for injectable solutions.

Pharmaceutically acceptable diluent or carrier include starch, glucose, lactose, sucrose, sodium stearate, glycerol monostearate, talc, sodium chloride, dried skim milk, glycerol, propylene glycol, water, ethanol and the like.

The pharmaceutical compositions may further contain one or more pharmaceutically acceptable salts such as, for example, a mineral acid salt such as a hydrochloride, a hydrobromide, a phosphate, a sulfate, etc.; and the salts of organic acids such as acetates, propionates, malonates, benzoates, etc. Additionally, auxiliary substances, such as wetting or emulsifying agents, pH buffering substances, gels or gelling materials, flavorings, colorants, microspheres, polymers, suspension agents, etc. may also be present herein. In addition, one or more other conventional pharmaceutical ingredients, such as preservatives, humectants, suspending agents, surfactants, antioxidants, anticaking agents, fillers, chelating agents, coating agents, chemical stabilizers, etc. may also be present, especially if the dosage form is a reconstitutable form. Suitable exemplary ingredients include macrocrystalline cellulose, carboxymethyf cellulose sodium, polysorbate 80, phenyletbyl alcohol, chiorobutanol, potassium sorbate, sorbic acid, sulfur dioxide, propyl gallate, the parabens, ethyl vanillin, glycerin, phenol, parachlorophenol, gelatin, albumin and a combination thereof. A thorough discussion of pharmaceutically acceptable excipients is available in REMINGTON'S PHARMACEUTICAL SCIENCES (Mack Pub. Co., N.J. 1991) which is incorporated by reference herein.

In some embodiments, the pharmaceutical acceptable carrier described herein is an agent (e.g. a molecule or a cell) that improves drug delivery properties of the agents for use of the invention. In some embodiments the drug delivery property describe herein comprises at least one property selected from the group of penetration ability (e.g. cell-membrane and/or blood brain barrier), site specific delivery (e.g. brain specific delivery), controlled release delivery and stability (e.g., reduction of enzymatic degradation). In some embodiments, the pharmaceutical carrier described herein is an agent selected from the group of delivery cell, liposome, nanoparticle, fusion protein, niosome, nanosphere, micelle, nanocapsule, nanoshell, lipid particle and dendrimer.

In one embodiment, the invention relates to a method of treatment comprising administering an effective amount of a pharmaceutical composition comprising AAT protein and/or a nucleic acid encoding AAT to a subject, wherein the subject is suffering from a disease or disorder of the nervous system and wherein the subject is undergoing a therapy comprising administration of a plurality of IgG antibodies, isoforms thereof, fragments thereof, and/or IgG variants.

An “effective amount” of an agent, e.g., a therapeutic agent, refers to an amount effective, at dosages and for periods of time necessary, to achieve the desired therapeutic or prophylactic result. Furthermore, the effective amount may depend on the individual patient's history, age, weight, family history, genetic makeup, stage of the thyroid-related autoimmune disease, the types of preceding or concomitant treatments, if any, and other individual characteristics of the subject to be treated.

In some cases, an effective amount of the compositions/compounds/products described herein can be any amount that reduces the severity, or occurrence, of symptoms of the disease, disorder and/or condition to be treated without producing significant toxicity to the subject. In some cases, an effective amount of the compositions/compounds/products described herein can be any amount that reduces the number of diseased cells, autoantibodies, and/or other disease markers (e.g. cytokines) without producing significant toxicity to the subject.

As used herein the terms “subject”/“subject in need thereof”, or “patient”/“patient in need thereof” are well-recognized in the art, and, are used interchangeably herein to refer to a mammal, including dog, cat, rat, mouse, monkey, cow, horse, goat, sheep, pig, camel, and, most preferably, a human. In some cases, the subject is a subject in need of treatment or a subject with a disease or disorder. In some embodiments, the subject to be treated is a subject above the age of 1, 2, 3, 4, 5, 6, 7, 8, 9, 10, or 12, preferably above the age of 1. According to certain embodiments, the subject is selected from the group consisting of a pre-pubertal child, a pre-pubertal adolescent, an adolescent and an adult. In some embodiments, the subject to be treated is female.

In one embodiment, the invention relates to a method of treatment comprising administering an effective amount of a pharmaceutical compound comprising a plurality of IgG antibodies, isoforms thereof, fragments thereof, and/or IgG variants to a subject, wherein the subject is suffering from a disease or disorder of the nervous system and wherein the subject is undergoing a therapy comprising administration of AAT protein and/or a nucleic acid encoding AAT.

In certain embodiments, the invention relates to the kit of parts for use of the invention, wherein the disease or disorder of the nervous system is chronic inflammatory demyelinating polyneuropathy.

In certain embodiments, the invention relates to the pharmaceutical composition for use of the invention, wherein the disease or disorder of the nervous system is chronic inflammatory demyelinating polyneuropathy.

In certain embodiments, the invention relates to the method of treatment of the invention, wherein the disease or disorder of the nervous system is chronic inflammatory demyelinating polyneuropathy.

In certain embodiments, the invention relates to the pharmaceutical product for use of the invention, wherein a subject to be treated has at least one symptom of chronic inflammatory demyelinating polyneuropathy or a history of at least one symptom of chronic inflammatory demyelinating polyneuropathy.

In certain embodiments, the invention relates to the kit of parts for use of the invention, wherein a subject to be treated has at least one symptom of chronic inflammatory demyelinating polyneuropathy or a history of at least one symptom of chronic inflammatory demyelinating polyneuropathy.

In certain embodiments, the invention relates to the pharmaceutical composition for use of the invention, wherein a subject to be treated has at least one symptom of chronic inflammatory demyelinating polyneuropathy or a history of at least one symptom of chronic inflammatory demyelinating polyneuropathy.

In certain embodiments, the invention relates to the method of treatment of the invention, wherein a subject to be treated has at least one symptom of chronic inflammatory demyelinating polyneuropathy or a history of at least one symptom of chronic inflammatory demyelinating polyneuropathy.

The term “symptom of chronic inflammatory demyelinating polyneuropathy”, as used herein, refers to at least one symptom selected from the group consisting of diminished or absent deep-tendon reflexes, sensory ataxia, weakness, numbness, tingling, pain, difficulty in walking, proximal muscle weakness in the limbs and distal muscle weakness in the limbs.

In certain embodiments, the invention relates to the kit of parts for use of the invention, wherein the plurality of IgG antibodies, isoforms thereof, fragments thereof, and/or IgG variants are recombinant IgG antibodies, isoforms thereof, fragments thereof, and/or IgG variants.

In certain embodiments, the invention relates to the pharmaceutical composition for use of the invention, wherein the plurality of IgG antibodies, isoforms thereof, fragments thereof, and/or IgG variants are recombinant IgG antibodies, isoforms thereof, fragments thereof, and/or IgG variants.

In certain embodiments, the invention relates to the method of treatment of the invention, wherein the plurality of IgG antibodies, isoforms thereof, fragments thereof, and/or IgG variants are plasma derived IgG antibodies, isoforms thereof, fragments thereof, and/or IgG variants.

In certain embodiments, the invention relates to the kit of parts for use of the invention, wherein the plurality of IgG antibodies, isoforms thereof, fragments thereof, and/or IgG variants are recombinant IgG antibodies, isoforms thereof, fragments thereof, and/or IgG variants.

In certain embodiments, the invention relates to the pharmaceutical composition for use of the invention, wherein the plurality of IgG antibodies, isoforms thereof, fragments thereof, and/or IgG variants are recombinant IgG antibodies, isoforms thereof, fragments thereof, and/or IgG variants.

In certain embodiments, the invention relates to the method of treatment of the invention, wherein the plurality of IgG antibodies, isoforms thereof, fragments thereof, and/or IgG variants are recombinant IgG antibodies, isoforms thereof, fragments thereof, and/or IgG variants.

In certain embodiments, the invention relates to the kit of parts for use of the invention, wherein the IgG antibodies, isoforms thereof, fragments thereof, and/or IgG variants are formulated for intravenous administration.

In certain embodiments, the invention relates to the pharmaceutical composition for use of the invention, wherein the IgG antibodies, isoforms thereof, fragments thereof, and/or IgG variants are formulated for intravenous administration.

In certain embodiments, the invention relates to the method of treatment of the invention, wherein the IgG antibodies, isoforms thereof, fragments thereof, and/or IgG variants are formulated for intravenous administration.

In certain embodiments, the invention relates to the kit of parts for use of the invention, wherein the IgG antibodies, isoforms thereof, fragments thereof, and/or IgG variants are formulated for subcutaneous administration.

In certain embodiments, the invention relates to the pharmaceutical composition for use of the invention, wherein the IgG antibodies, isoforms thereof, fragments thereof, and/or IgG variants are formulated for subcutaneous administration.

In certain embodiments, the invention relates to the method of treatment of the invention, wherein the IgG antibodies, isoforms thereof, fragments thereof, and/or IgG variants are formulated for subcutaneous administration.

In certain embodiments, the invention relates to the pharmaceutical product for use of the invention, wherein the AAT protein is recombinant AAT.

In certain embodiments, the invention relates to the kit of parts for use of the invention, wherein the AAT protein is recombinant AAT.

In certain embodiments, the invention relates to the pharmaceutical composition for use of the invention, wherein the AAT protein is recombinant AAT.

In certain embodiments, the invention relates to the method of treatment of the invention, wherein the AAT protein is recombinant AAT.

In certain embodiments, the invention relates to the pharmaceutical product for use of the invention, wherein the AAT protein is plasma derived AAT.

In certain embodiments, the invention relates to the kit of parts for use of the invention, wherein the AAT protein is plasma derived AAT.

In certain embodiments, the invention relates to the pharmaceutical composition for use of the invention, wherein the AAT protein is plasma derived AAT.

In certain embodiments, the invention relates to the method of treatment of the invention, wherein the AAT protein is plasma derived AAT.

In certain embodiments, the invention relates to the pharmaceutical product for use of the invention, wherein a) the alpha1-antitrypsin (AAT) protein, variant, isoform and/or a fragment thereof, wherein said variant, isoform and/or fragment has inhibitory activity; and/or b) the nucleic acid encoding AAT, variant, isoform and/or fragment thereof having ADAM17 inhibitory activity is/are formulated for intravenous administration.

In certain embodiments, the invention relates to the kit of parts for use of the invention, wherein a) the alpha1-antitrypsin (AAT) protein, variant, isoform and/or a fragment thereof, wherein said variant, isoform and/or fragment has inhibitory activity; and/or b) the nucleic acid encoding AAT, variant, isoform and/or fragment thereof having ADAM17 inhibitory activity is/are formulated for intravenous administration.

In certain embodiments, the invention relates to the pharmaceutical composition for use of the invention, wherein a) the alpha1-antitrypsin (AAT) protein, variant, isoform and/or a fragment thereof, wherein said variant, isoform and/or fragment has inhibitory activity; and/or b) the nucleic acid encoding AAT, variant, isoform and/or fragment thereof having ADAM17 inhibitory activity is/are formulated for intravenous administration.

In certain embodiments, the invention relates to the method of treatment of the invention, wherein a) the alpha1-antitrypsin (AAT) protein, variant, isoform and/or a fragment thereof, wherein said variant, isoform and/or fragment has inhibitory activity; and/or b) the nucleic acid encoding AAT, variant, isoform and/or fragment thereof having ADAM17 inhibitory activity is/are formulated for intravenous administration.

In certain embodiments, the invention relates to the pharmaceutical product for use of the invention, wherein a) the alpha1-antitrypsin (AAT) protein, variant, isoform and/or a fragment thereof, wherein said variant, isoform and/or fragment has inhibitory activity; and/or b) the nucleic acid encoding AAT, variant, isoform and/or fragment thereof having ADAM17 inhibitory activity is/are formulated for subcutaneous administration.

In certain embodiments, the invention relates to the kit of parts for use of the invention, wherein a) the alpha1-antitrypsin (AAT) protein, variant, isoform and/or a fragment thereof, wherein said variant, isoform and/or fragment has inhibitory activity; and/or b) the nucleic acid encoding AAT, variant, isoform and/or fragment thereof having ADAM17 inhibitory activity is/are formulated for subcutaneous administration.

In certain embodiments, the invention relates to the pharmaceutical composition for use of the invention, wherein a) the alpha1-antitrypsin (AAT) protein, variant, isoform and/or a fragment thereof, wherein said variant, isoform and/or fragment has inhibitory activity; and/or b) the nucleic acid encoding AAT, variant, isoform and/or fragment thereof having ADAM17 inhibitory activity is/are formulated for subcutaneous administration.

In certain embodiments, the invention relates to the method of treatment of the invention, wherein a) the alpha1-antitrypsin (AAT) protein, variant, isoform and/or a fragment thereof, wherein said variant, isoform and/or fragment has inhibitory activity; and/or b) the nucleic acid encoding AAT, variant, isoform and/or fragment thereof having ADAM17 inhibitory activity is/are formulated for subcutaneous administration.

The present invention also contemplates a gene delivery vector encoding AAT (or an isoform, a fragment or variant thereof) and pharmaceutical compositions containing the same. The gene delivery vector may be used for any of the therapeutical uses described herein. Preferably, the gene delivery vector is in the form of a plasmid or a vector that comprises one or more nucleic acid encoding the AAT protein, a variant, an isoform and/or a fragment thereof of the invention. Examples of gene delivery vectors comprise e.g., viral vectors, non-viral vectors, particulate carriers, and liposomes. The gene deliver is preferably performed in vitro or ex vivo.

The kits, pharmaceutical compositions and/or products of the present invention may be administered to a subject by different routes including orally, parenterally, sublingually, transdermally, rectally, transmucosally, topically, via inhalation, via buccal administration, intrapleurally, intravenous, intraarterial, intraperitoneal, subcutaneous, intramuscular, intranasal intrathecal, and intraarticular or combinations thereof. For human use, the composition may be administered as a suitably acceptable formulation in accordance with normal human practice. The person skilled in the art will readily determine the dosing regimen and route of administration that is most appropriate for a particular patient. The kits, pharmaceutical compositions and/or products of the invention may be administered by traditional syringes, needleless injection devices, “microprojectile bombardment gone guns”, or other physical methods such as electroporation (“EP”), “hydrodynamic method”, or ultrasound. The composition can also be administered by intravenous injection, intravenous infusion, infusion with a dosator pump, inhalation nasal-spray, eye-drops, skin-patches, slow release formulations, ex vivo gene therapy or ex vivo cell-therapy, preferably by intravenous injection.

The pharmaceutical compositions of the present invention may also be delivered to the patient, by several technologies including DNA injection of nucleic acid encoding the AAT protein, a variant, an isoform and/or a fragment thereof of the invention (also referred to as DNA vaccination) with and without in vivo electroporation, liposome mediated, nanoparticle facilitated, recombinant vectors such as recombinant lentivirus, recombinant adenovirus, and recombinant adenovirus associated virus as described herein.

All definitions and combinations provided herein apply to these embodiments, if applicable and unless indicated otherwise.

Although methods and materials similar or equivalent to those described herein can be used in the practice or testing of the present invention, suitable methods and materials are described below. All publications, patent applications, patents, and other references mentioned herein are incorporated by reference in their entirety. The publications and applications discussed herein are provided solely for their disclosure prior to the filing date of the present application. Nothing herein is to be construed as an admission that the present invention is not entitled to antedate such publication by virtue of prior invention. In addition, the materials, methods, and examples are illustrative only and are not intended to be limiting.

In the case of conflict, the present specification, including definitions, will control. Unless defined otherwise, all technical and scientific terms used herein have the same meaning as is commonly understood by one of skill in art to which the subject matter herein belongs. As used herein, the following definitions are supplied in order to facilitate the understanding of the present invention.

The term “about” particularly in reference to a given quantity, is meant to encompass deviations of plus or minus 20 percent, preferably 10 percent, preferably 5 percent, even more preferably 2 percent and most preferably 1 percent.

As used in the specification and claims, the singular form “a”, “an” and “the” include plural references unless the context clearly dictates otherwise.

As used herein, “at least one” means “one or more”, “two or more”, “three or more”, etc. “or” should be understood to mean either one, both, or any combination thereof of the alternatives.

“and/or” should be understood to mean either one, or both of the alternatives.

Throughout this specification, unless the context requires otherwise, the words “comprise”, “comprises” and “comprising” will be understood to imply the inclusion of a stated step or element or group of steps or elements but not the exclusion of any other step or element or group of steps or elements.

The terms “include” and “comprise” are used synonymously. “preferably” means one option out of a series of options not excluding other options. “e.g.” means one example without restriction to the mentioned example. By “consisting of” is meant including, and limited to, whatever follows the phrase “consisting of.”

Reference throughout this specification to “one embodiment”, “an embodiment”, “a particular embodiment”, “a related embodiment”, “a certain embodiment”, “an additional embodiment”, “some embodiments”, “a specific embodiment” or “a further embodiment” or combinations thereof means that a particular feature, structure or characteristic described in connection with the embodiment is included in at least one embodiment of the present invention. Thus, the appearances of the foregoing phrases in various places throughout this specification are not necessarily all referring to the same embodiment. Furthermore, the particular features, structures, or characteristics may be combined in any suitable manner in one or more embodiments. It is also understood that the positive recitation of a feature in one embodiment, serves as a basis for excluding the feature in a particular embodiment.

Unless otherwise defined, all technical and scientific terms used herein have the same meaning as commonly understood by one of ordinary skill in the art to which this invention pertains.

Although methods and materials similar or equivalent to those described herein can be used in the practice or testing of the present invention, suitable methods and materials are described below. In case of conflict, the present specification, including definitions, will control. In addition, the materials, methods, and examples are illustrative only and not intended to be limiting.

The general methods and techniques described herein may be performed according to conventional methods well known in the art and as described in various general and more specific references that are cited and discussed throughout the present specification unless otherwise indicated. See, e.g., Sambrook et al., Molecular Cloning: A Laboratory Manual, 2d ed., Cold Spring Harbor Laboratory Press, Cold Spring Harbor, N.Y. (1989) and Ausubel et al., Current Protocols in Molecular Biology, Greene Publishing Associates (1992), and Harlow and Lane Antibodies: A Laboratory Manual, Cold Spring Harbor Laboratory Press, Cold Spring Harbor, N.Y. (1990).

While embodiments of the invention are illustrated and described in detail in the figures and foregoing description, such illustration and description are to be considered illustrative or exemplary and not restrictive. It will be understood that changes and modifications may be made by those of ordinary skill within the scope and spirit of the following claims. In particular, the present invention covers further embodiments with any combination of features from different embodiments described above and below.

### BRIEF DESCRIPTION OF THE DRAWINGS

FIGS. 1A and 1B: IFNγ-mediated microglial activation

FIGS. 2A and 2B: AAT decreases IFNγ-mediated microglial activation

FIGS. 3A, 3B and 3C: Validation of AAT anti-inflammatory effect on the experiment used for RNAseq

FIGS. 4A, 4B and 4C: Validation of microglial activation and AAT anti-inflammatory effect by GSEA analysis

FIGS. 5A, 5B and 5C: AAT (Sigma Aldrich, batch A6150) inhibits TACE activity in a cell-free assay shown as relative fluorescence unit (FIG. 5A) or as percentage of the control activity (FIG. 5B) with an IC₅₀ of 15.3 μM (FIG. 5C)

FIGS. 6A and 6B: Results of the sciatic nerve electrophysiology (EMG) (FIG. 6A) Amplitude (FIG. 6A B) conduction velocity

FIGS. 7A and 7B: Results of the grip strength test (FIG. 7A) absolute values, (FIG. 7B) % of 6 week timepoint

FIGS. 8A and 8B: Results of the rotarod test (FIG. 8A) absolute values, (FIG. 8B) % of 6 week timepoint

FIG. 9: DNAJB9 and PLA2G4B gene expression in response to AAT treatment

FIG. 10: Number of axons

FIG. 11: Axonal diameter

FIG. 12: g ratio

FIGS. 13A,13B and 13C: Individual histology images of sciatic nerve semithin cross sections. Scale bar 10 μm FIG. 13A) Group 1: WT control, FIG. 13B) CMT1A+vehicle, FIG. 13C) CMT1A+AAT

FIG. 14: Plasma IL-6 concentration

FIG. 15: Plasma TNFα concentration

FIG. 16: Study scheme for CMT1A mouse model and AAT administration

FIGS. 17A and 17B: Cell morphology and count after treatments: SH-SY5Y morphological analysis and cell count after 6-OHDA administration and AAT treatment. Brightfield pictures (20×) and cell count (D0 vs D4 of culture) showing the effect of treatments on SH-SY5Y cell phenotype and proliferation and AAT positive action. FIG. 17A) Example images FIG. 17B) Quantification

FIG. 18: Cell viability: Graph represents cell viability measured through absorbance (450 nm) for control and samples

FIG. 19: IL-6 quantification in cell supernatant

FIG. 20: Study scheme

FIGS. 21A, 21B and 21C: Neuromuscular tests: FIG. 21A Rotarod latency; FIG. 21B Grip strength; FIG. 21C Von Frey test

FIGS. 22A, and 22B: Sciatic nerve electrophysiology: FIG. 22A: Compound muscle action potential; FIG. 22B: Nerve conduction velocity

FIGS. 23A and 23B: FIG. 23A) Graphical representation of plasma TMPRSS5 FIG. 23B) Graphical representation of plasma NfL conc

FIG. 24: Graphical representation of plasma TNFα concentration

FIG. 25: Rotarod latency to fall of CMT mice treated with AAT (180 mg/kg sc)

FIG. 26: Grip strength (Newton) of CMT mice treated with AAT (180 mg/kg sc)

FIG. 27: CMAP (mV) of CMT mice treated with AAT (180 mg/kg sc)

FIG. 28: NCV (m/s) of CMT mice treated with AAT (180 mg/kg sc)

FIG. 29: Plasma IL-6 concentration

FIG. 30: Plasma TNFα concentration

FIGS. 31A and 31B: Effect of peptide 8 on ADAM17 activity. (***, ****: P<0.001, P<0.0001, one-way ANOVA (multiple comparison); ns=not significant vs DMSO

FIG. 32: Effect of AAT on the gene expression of the NF-κB pathway following TNFα-dependent activation of Schwann Cells

FIG. 33: Effect of AAT on the gene expression related to the oxidative stress response following TNFα-dependent activation of Schwann Cells

FIG. 34: Effect of AAT on the gene expression related to the NRF2-related oxidative stress response following TNFα-dependent activation of Schwann Cells

FIG. 35: Effect of AAT and its derivative peptides on TNF induced Schwann cells.

FIG. 36: Number of axons/100 μm²

FIG. 37: axonal diameter

FIG. 38: g-ratio

FIGS. 39A, 39B and 39C: FIG. 39A) Number of axons/100 μm² FIG. 39B) Axonal diameter and FIG. 39C) g-ratio

FIG. 40: Plasma NfL concentration

FIG. 41: Effect of AAT and its derivative peptides in IFNγ-induced microglia

FIG. 42: Synthesis of peptide #8 and #9 (Synthesis: Syro-1 automatic peptide synthesizer; Double coupling conditions: DIC (4 eq)/Oxyma (4 eq), 40 min; HATU (4 eq)/DIPEA (8 eq), 30 min; Deprotection: 40% piperidine in DMF, 3 min and 20% piperidine in DMF, 12 min)

FIG. 43: Synthesis peptidomimetic #14 (Synthesis: Manual Synthesis; Coupling condition: DIC (3 eq)/Oxyma (3 eq), 2 h; Deprotection: 20% piperidine in DMF, 5 min; 20% piperidine in DMF, 20 min)

### DETAILED DESCRIPTION OF THE INVENTION

### Examples

Those skilled in the art will appreciate that the invention described herein is susceptible to variations and modifications other than those specifically described. It is to be understood that the invention includes all such variations and modifications without departing from the spirit or essential characteristics thereof. The invention also includes all of the steps, features, compositions and compounds referred to or indicated in this specification, individually or collectively, and any and all combinations or any two or more of said steps or features. The present disclosure is therefore to be considered as in all embodiments illustrated and not restrictive, the scope of the invention being indicated by the appended claims, and all changes which come within the meaning and range of equivalency are intended to be embraced therein. Various references are cited throughout this Specification, each of which is incorporated herein by reference in its entirety. The foregoing description will be more fully understood with reference to the following Examples.

### Example 1

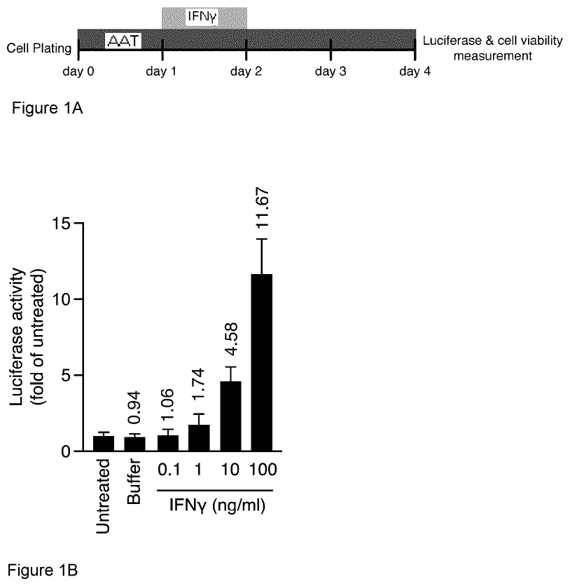

A) Human microglial cells HMC3-MHCII^{Luc} cells were plated at day 0, activated with IFNγ at day1 until day 2 and measurement of the luciferase activity and cell viability were done at day 4.
B) Activation was measured by the activity of MHCII-driven luciferase and normalized to cell viability. Luciferase activity for all conditions is represented as fold of the untreated control. A potential effect of the highest concentration of the buffer used for drug presentation (IFNγ, AATs) was precluded. All conditions were performed in triplicates, error bars represent standard deviation (FIGS. 1A and 1B).

### Example 2

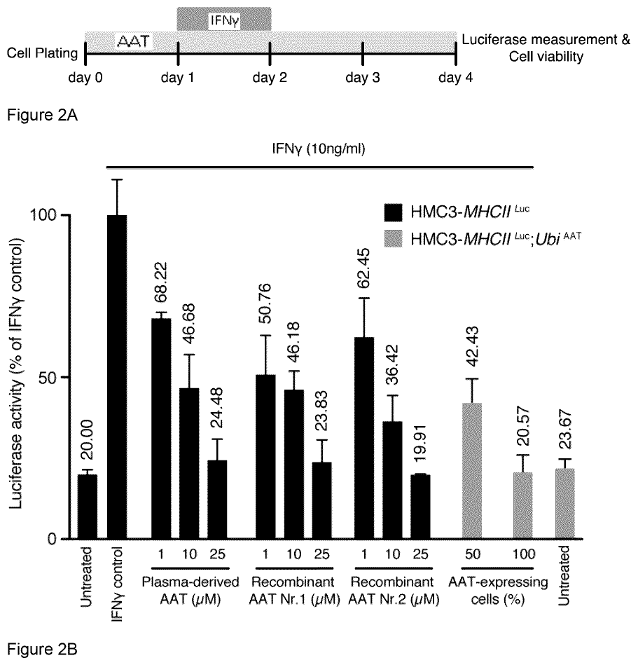

A) Human microglial HMC3-MHCII^{Luc} and HMC3MHCII^{Luc}; Ubi^{AAT} cells were plated at day 0, presented IFNγ at day 1 until day 2 and measurement of the luciferase activity and cell viability were done at day 4.
B) AATs were applied from day 0 to day 4 on HMC3-MHCII^{Luc} cells. Activation was measured by the activity of MHCII-driven luciferase and normalized to cell viability. Luciferase activity for all conditions is represented as percentage of IFNγ control. All conditions were performed in triplicates, error bars represent standard deviation. (FIGS. 2A and 2B)

### Example 3

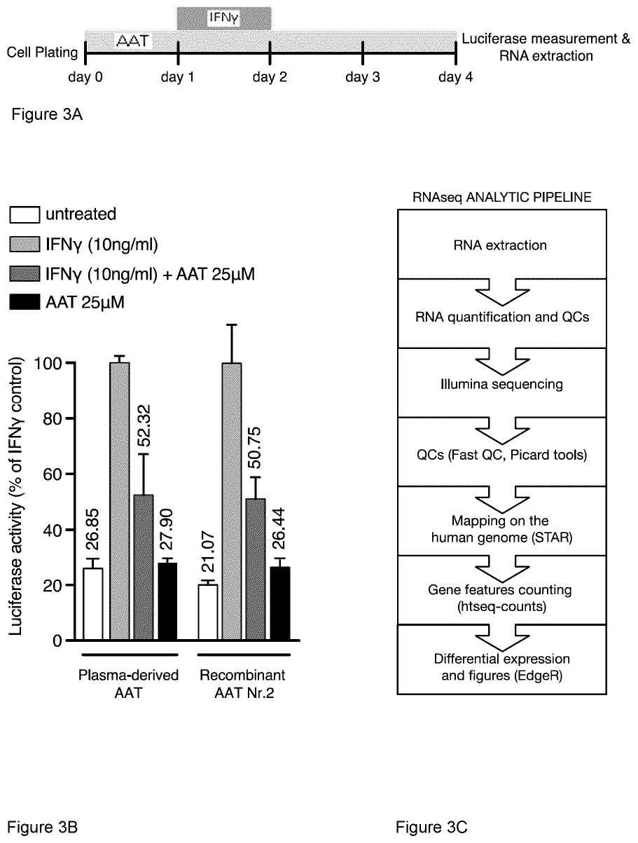

A) Human microglial HMC3-MHCII^{Luc} cells were plated at day 0, presented IFNγ at day 1 until day 2 and measurement of the luciferase activity and cell viability were done at day 4.
B) AATs were applied from day 0 to day 4 on HMC3-MHCII^{Luc} cells. Activation was measured by the activity of MHCII-driven luciferase and represented as percentage of IFNγ control for all conditions. All conditions were performed in triplicates, error bars represent standard deviation.
C) Bulk RNA extraction was performed on the same HMC3-MHCII^{Luc} cultures. Quality control (QC) were applied to RNA before sequencing. QC of the sequencing was done prior to mapping on the human genome. Mapped reads were counted, and differential gene expression was measured between the conditions (see Table 1-10). (FIGS. 3A, 3B and 3C)

### Example 4

RNAseq data from plasma-derived and recombinant AATs were pooled, normalized and analyzed through Gene Set Enrichment Analysis (GSEA) (https://www.gsea-msigdb.org/gsea/index.jsp; Subramanian, A., et al. Proc. Natl. Acad. Sci. USA, 102 (43): 15545-15550, 2005.). Upregulated gene families (grey bars) and downregulated ones (black bars) are shown according to normalized enrichment score.
A) The inflammatory profile induced by IFNγ was confirmed by upregulation of several processes related to inflammation (bold italic).
B-C) AAT treatment, in absence (B) or presence (C) of IFNγ was able to significantly downregulate some of these pathways (bold italic). Importantly, both the IFNγ and inflammatory responses were dampened by AAT. However, the downregulation of the inflammatory response genes in (C) was just under significance.

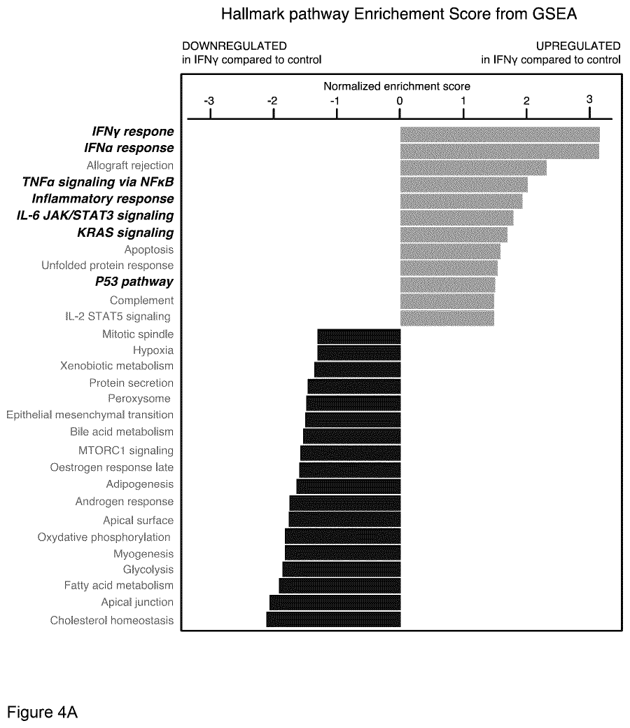

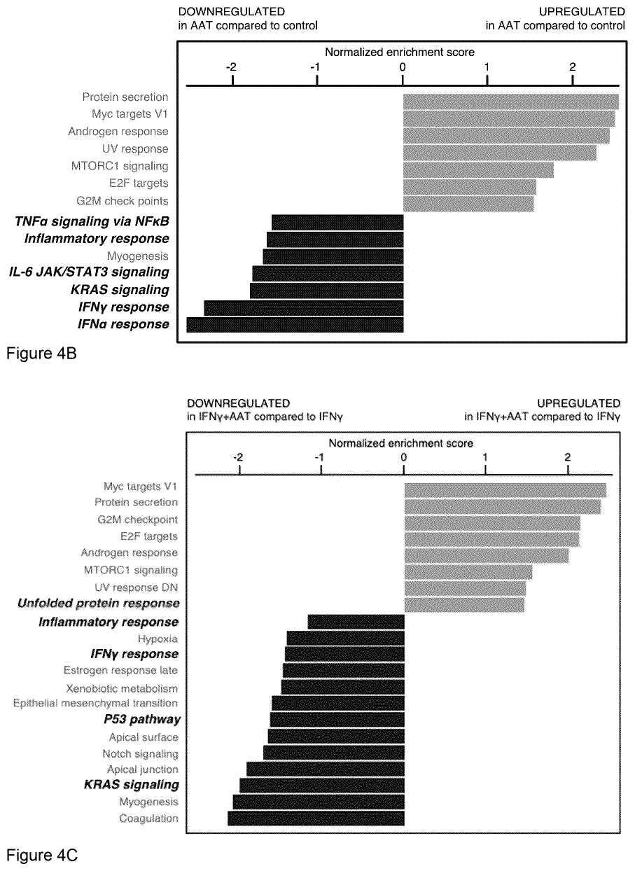

Regarding other gene families, KRAS signaling is of importance since it has been involved in oncogenic process and immunomodulation (Dias Carvalho, P., et al., 2018, Cancer Res, 78: 7-14.). As well, p53 pathway is of note as a mediator of response to stress. (FIGS. 4A, 4B and 4C)

### Example 5

### AAT Treatment Counterbalances the Expression of Inflammatory Genes

Tables of genes related to antigen presentation (Table 1), cytokine signaling (Table 2), interferon signaling (Table 3) and complement activation (Table 4). Inflammatory top gene (FC>2; p-value<0.05; left column), were defined by differential expression between untreated and IFNγ-treated cells (inflammation; middle column). Top genes that were significantly and oppositely regulated by AAT treatment are highlighted (right column; bold underline).

A substantial number (˜35%) of inflammatory top genes were found affected by AAT treatment (6 of 23 genes related to antigen presentation; 14 of 33 genes related to cytokine signaling; 1 of 7 genes related to complement activation; 5/13 genes related to interferon signaling) showing its anti-inflammatory potential. For example, the promoter of HLA-DRA gene which was used as a driver for luciferase expression in the HMC3-MHCII^{Luc} line was consistently up- and down-regulated in inflammation and with AAT treatment, respectively (bold italic). Of note, FCs shown in AAT-treated inflammation condition should not be directly compared to those found in inflammation since for the former, a FC=2 represents already a 50% counterbalance on inflammation induced/repressed genes.

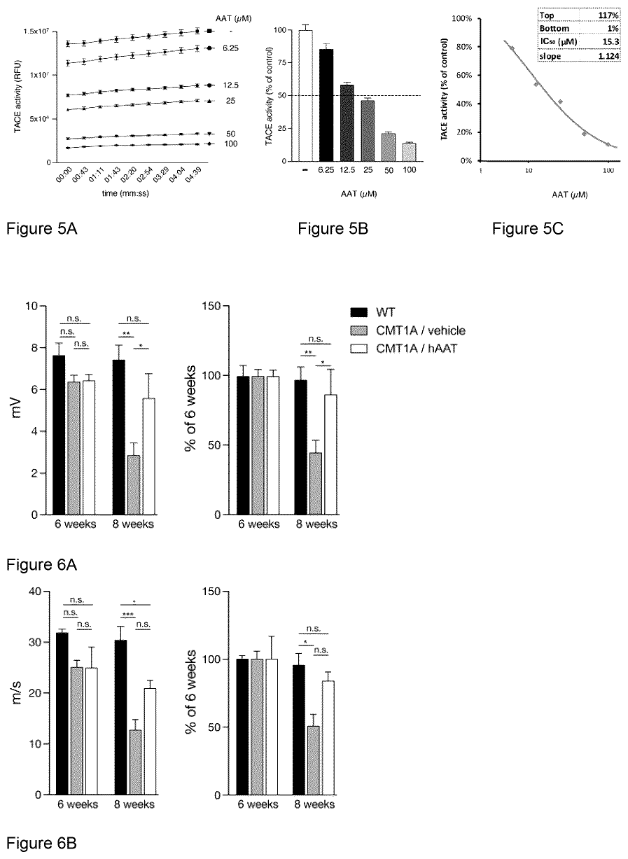

Second order inflammatory genes (p-value<0.05; FC<2) significantly and oppositely regulated by AAT treatment in inflammatory condition or resting condition (regulated in AAT) are presented at the bottom of the table. (FIGS. 5A, 5B and 5C)

TABLE 1
Antigen presentation

| Top Gene | p-value | FC | p-value | FC |
|---|---|---|---|---|
| | | | DOWN in | |
| | UP in inflammation | | inflammation + AAT | |
| CD74 | 4.15E−14 | 75.81 | | |
| LAMP3 | 9.39E−12 | 7.94 | | |
| TAP1 | 5.99E−11 | 6.12 | | |
| PSMB9 | 5.73E−11 | 5.96 | 3.11E−02 | −1.23 |
| HLA-DOB | 5.11E−04 | 4.48 | 6.82E−03 | −4.49 |
| PSMB8 | 5.18E−11 | 4.22 | | |
| HLA-DRA | 4.99E−10 | 4.76 | 4.68E−02 | −2.22 |
| SOCS1 | 1.21E−05 | 3.99 | | |
| TAP2 | 8.97E−13 | 3.54 | | |
| NCF2 | 2.57E−05 | 2.88 | | |
| CTSS | 2.29E−05 | 3.71 | | |
| HLA-DPA1 | 8.31E−09 | 3.22 | | |
| BATF3 | 1.45E−06 | 2.55 | | |
| NFKB1 | 6.21E−10 | 2.41 | | |
| IL1B | 5.54E−04 | 2.08 | 1.11E−03 | −2.07 |
| FYN | 2.18E−07 | 1.80 | | |
| BCL10 | 3.21E−05 | 1.65 | | |
| MALT1 | 1.75E−04 | 1.62 | | |
| | DOWN in inflammation | | UP in inflammation + AAT | |
| TUBA1A | 2.29E−12 | −5.00 | | |
| KIF20A | 7.29E−12 | −3.75 | | |
| CALR | 6.15E−11 | −3.53 | 4.74E−04 | 1.35 |
| PYCARD | 6.92E−03 | −2.86 | 1.53E−02 | 2.20 |
| CDC20 | 1.05E−08 | −2.80 | | |

| | p-value | FC | p-value | FC |
|---|---|---|---|---|
| Other Gene | DOWN in inflammation | | UP in inflammation + AAT | |
| NFATC2 | <0.05 | −1.37 | <0.05 | 1.63 |
| MRC2 | <0.05 | −1.61 | <0.05 | 1.47 |
| CTSD | <0.05 | −1.34 | <0.05 | 1.47 |
| HLA-DPB1 | <0.05 | −1.23 | <0.05 | 1.31 |
| TAPBP | <0.05 | −1.90 | <0.05 | 1.23 |
| SOCS3 | <0.05 | −1.99 | <0.05 | 1.20 |
| CTSA | <0.05 | −1.31 | <0.05 | 1.17 |
| | Regulated in inflammation | | Regulated in AAT | |
| NFATC1 | <0.05 | −1.29 | <0.05 | 1.44 |
| TRAF6 | <0.05 | 1.08 | <0.05 | −1.26 |

TABLE 2
Cytokine signalling

| Top Gene | p-value | FC | p-value | FC |
|---|---|---|---|---|
| | | | DOWN in | |
| | UP in inflammation | | inflammation + AAT | |
| IL6 | 3.53E−10 | 17.139 | | |
| TNFSF10 | 2.53E−07 | 10.595 | | |
| TRAF1 | 4.39E−07 | 7.789 | | |
| CCL5 | 5.17E−09 | 7.540 | | |
| ATF3 | 6.86E−13 | 7.218 | 1.85E−03 | −1.26 |
| PSMB9 | 6.09E−10 | 5.963 | 3.11E−02 | −1.23 |
| CEBPB | 2.57E−10 | 5.808 | | |
| CSF1 | 3.52E−09 | 5.455 | | |
| PSMB8 | 1.63E−10 | 4.219 | | |
| IL23A | 6.49E−03 | 4.218 | | |
| STAT1 | 2.04E−09 | 4.110 | | |
| IL15RA | 4.63E−07 | 3.660 | | |
| FGF2 | 1.66E−06 | 3.608 | 9.13E−03 | −1.57 |
| CXCL2 | 6.42E−05 | 3.467 | | |
| IL21R | 1.16E−05 | 3.235 | | |
| CEBPG | 3.19E−09 | 2.909 | | |
| CXCL1 | 1.36E−04 | 2.82 | 7.22E−03 | −2.09 |
| CASP1 | 1.07E−05 | 2.740 | | |
| TNFRSF9 | 2.39E−04 | 2.434 | 1.23E−03 | −2.35 |
| CXCL5 | 5.07E−05 | 2.36 | 5.98E−03 | −2.15 |
| IL1B | 2.14E−03 | 2.08 | 1.11E−03 | −2.07 |
| | DOWN in inflammation | | UP in inflammation + AAT | |
| FSCN1 | 9.42E−08 | −6.312 | 8.09E−04 | 1.48 |
| CCL2 | 2.95E−03 | −6.086 | | |
| IL411 | 3.22E−03 | −4.19 | 3.22E−03 | 1.95 |
| IL27RA | 2.22E−07 | −3.848 | 2.67E−02 | 1.36 |
| STAT5A | 7.66E−04 | −3.53 | 9.78E−03 | 1.45 |
| CEBPD | 5.07E−05 | −3.102 | | |
| EGF | 6.13E−03 | −2.973 | | |
| FGF18 | 2.11E−05 | −2.754 | | |
| IL6R | 6.00E−08 | −2.630 | | |
| IL1R1 | 1.27E−07 | −2.566 | 3.94E−02 | 1.18 |
| JAK3 | 3.03E−08 | −2.43 | 3.20E−03 | 1.43 |
| RASAL1 | 1.36E−06 | −2.34 | 4.65E−03 | 1.52 |

| Other Gene | p-value | FC | p-value | FC |
|---|---|---|---|---|
| | | | DOWN in | |
| | UP in inflammation | | inflammation + AAT | |
| IL18 | <0.05 | 1.83 | <0.05 | −1.75 |
| NFKBIZ | <0.05 | 1.94 | <0.05 | −1.37 |
| ANXA1 | <0.05 | 1.17 | <0.05 | −1.33 |
| HBEGF | <0.05 | 1.36 | <0.05 | −1.30 |
| DUSP6 | <0.05 | 1.96 | <0.05 | −1.26 |
| | DOWN in inflammation | | UP in inflammation + AAT | |
| CX3CL1 | <0.05 | −1.38 | <0.05 | 1.61 |
| MAP2K2 | <0.05 | −1.28 | <0.05 | 1.51 |
| MAPK11 | <0.05 | −1.22 | <0.05 | 1.44 |
| TRAF2 | <0.05 | −1.66 | <0.05 | 1.42 |
| MAP2K3 | <0.05 | −1.56 | <0.05 | 1.37 |
| MAPK12 | <0.05 | −1.14 | <0.05 | 1.33 |
| MAPK13 | <0.05 | −1.27 | <0.05 | 1.23 |
| SOCS3 | <0.05 | −1.99 | <0.05 | 1.20 |
| | Regulated in inflammation | | Regulated in AAT | |
| BIRC2 | <0.05 | 1.53 | | −1.53 |
| STAT1 | <0.05 | 4.11 | | −1.47 |
| RASGRF2 | <0.05 | 1.32 | | −1.45 |
| MYD88 | <0.05 | 1.09 | | −1.35 |
| CEBPG | <0.05 | 2.91 | | −1.31 |
| RIPK2 | <0.05 | 2.03 | | −1.30 |
| TRAF6 | <0.05 | 1.14 | | −1.26 |
| SPTBN1 | <0.05 | 1.13 | | −1.21 |
| TM7SF3 | <0.05 | 1.15 | | −1.18 |
| TNFSF9 | <0.05 | −1.92 | | 1.26 |

TABLE 3
Interferon signaling

| | | | | |
|---|---|---|---|---|
| Top Gene | p-value | FC | p-value | FC |

| | DOWN in |
|---|---|
| UP in inflammation | inflammation + AAT |

| | | | | |
|---|---|---|---|---|
| MX2 | 7.26E−09 | 21.98 | 3.69E−03 | −2.14 |
| MX1 | 1.09E−09 | 19.39 | 3.90E−03 | −2.00 |
| IRF1 | 8.56E−12 | 8.63 | | |
| IFIT1 | 9.65E−11 | 6.73 | 3.11E−03 | −1.78 |
| ISG15 | 4.97E−06 | 5.27 | | |
| HLA-DRA | 5.04E−07 | 4.76 | 4.68E−02 | −2.22 |
| USP18 | 5.44E−08 | 4.68 | 3.59E−03 | −1.76 |
| PSMB8 | 1.63E−10 | 4.22 | | |
| STAT1 | 2.04E−09 | 4.11 | | |
| SOCS1 | 8.94E−06 | 3.99 | | |
| HLA-DPA1 | 7.92E−07 | 3.22 | | |
| IRF7 | 1.84E−04 | 2.83 | | |
| IRF5 | 2.36E−04 | 2.11 | | |

| Other Gene | p-value | FC | p-value | FC |
|---|---|---|---|---|
| | | | DOWN in | |
| | UP in inflammation | | inflammation + AAT | |
| IFNAR1 | <0.05 | 1.47 | <0.05 | −1.63 |
| FLNB | <0.05 | 1.25 | <0.05 | −1.20 |
| | DOWN in inflammation | | UP in inflammation + AAT | |
| IRF3 | <0.05 | −1.45 | <0.05 | 1.56 |
| HLA-DPB1 | <0.05 | −1.23 | <0.05 | 1.31 |
| SOCS3 | <0.05 | −1.99 | <0.05 | 1.20 |
| CD44 | <0.05 | −1.14 | <0.05 | 1.14 |
| | Regulated in inflammation | | Regulated in AAT | |
| STAT1 | <0.05 | 4.11 | <0.05 | −1.47 |
| IRF2 | <0.05 | 1.24 | <0.05 | −1.17 |

TABLE 4
Complement activation

| Top Gene | p-value | FC | p-value | FC |
|---|---|---|---|---|
| | | | DOWN in | |
| | UP in inflammation | | inflammation + AAT | |
| SERPINB2 | 1.47E−05 | 25.49 | 6.63E−06 | −4.63 |
| C5AR1 | 1.78E−10 | 22.55 | | |
| C5 | 2.05E−07 | 4.22 | | |
| THBD | 1.89E−04 | 2.81 | | |
| C3 | 1.97E−06 | 2.34 | | |
| PLAU | 5.48E−09 | 2.17 | | |
| | DOWN in inflammation | | UP in inflammation + AAT | |
| PROS1 | 2.84E−08 | −3.43 | | |

| Other Gene | p-value | FC | p-value | FC |
|---|---|---|---|---|
| | | | DOWN in | |
| | UP in inflammation | | inflammation + AAT | |
| C3AR1 | <0.05 | 2.44 | <0.05 | −1.50 |
| | DOWN in inflammation | | UP in inflammation + AAT | |
| PLAUR | <0.05 | −1.84 | <0.05 | 1.32 |
| ITGAX | <0.05 | −1.60 | <0.05 | 1.41 |
| | Regulated in inflammation | | Regulated in AAT | |
| PLAU | <0.05 | 2.17 | <0.05 | −1.27 |

### Example 6—AAT Treatment Enhances the Expression of Hallmark Genes Related to M2 Anti-Inflammatory Microglia

M2 microglia gene expression is promoted following a M2-type induction (FC study; Satoh 2017). AAT treatment in resting microglia (FC AAT) and in activated microglia (FC inflammation+AAT) was able to similarly enhance expression of M2 genes, while at lower magnitude. ˜60 of the modified genes were common among AAT treatment conditions (Table 5 in bold).

TABLE 5

| | | | | | FC |
|---|---|---|---|---|---|
| | FC | FC | M2 | FC | inflammation + |
| M2 Gene | study | AAT | Gene | study | AAT |
| MMP1 | 7.36 | 2.51 | HLA-DRB5 | 53.99 | 2.09 |
| ITGA11 | 4.78 | 1.65 | ANPEP | 8.90 | 1.25 |
| COL1A1 | 4.38 | 1.58 | THBS1 | 8.16 | 1.34 |
| TAGLN | 2.79 | 1.37 | MMP1 | 7.36 | 2.39 |
| GUK1 | 2.76 | 1.57 | BGN | 6.68 | 1.27 |
| PAPPA | 2.74 | 1.24 | ITGA11 | 4.78 | 1.54 |
| PLTP | 2.52 | 1.28 | OLFML2B | 4.78 | 1.44 |
| NTN4 | 2.38 | 1.25 | GUK1 | 2.76 | 1.31 |
| FOXC1 | 2.37 | 1.26 | FAT1 | 2.75 | 1.34 |
| PTPRS | 2.34 | 1.27 | PAPPA | 2.74 | 1.31 |
| COL11A1 | 2.30 | 1.14 | KDM2B | 2.69 | 1.17 |
| STC2 | 2.29 | 1.42 | CLMP | 2.46 | 1.22 |
| MXRA8 | 2.28 | 1.40 | PTPRS | 2.34 | 1.23 |
| TEKT4P2 | 2.17 | 2.46 | COL11A1 | 2.30 | 1.16 |
| SART1 | 2.11 | 1.44 | STC2 | 2.29 | 1.32 |
| FZD2 | 2.09 | 1.39 | MXRA8 | 2.28 | 1.49 |
| AP2A2 | 2.04 | 1.26 | TGFBI | 2.19 | 1.18 |
| FEZ1 | 1.99 | 1.45 | LOXL2 | 2.10 | 1.25 |
| ACAN | 1.98 | 1.37 | FZD2 | 2.09 | 1.37 |
| TGFB1I1 | 1.94 | 1.31 | FEZ1 | 1.99 | 1.34 |
| NID1 | 1.90 | 1.16 | AHNAK2 | 1.96 | 1.40 |
| RASA4B | 1.87 | 1.46 | TGFB1I1 | 1.94 | 1.26 |
| GREM1 | 1.82 | 1.69 | CLIP3 | 1.89 | 1.25 |
| FGD3 | 1.82 | 1.76 | SH3PXD2B | 1.88 | 1.19 |
| GADD45GIP1 | 1.70 | 1.55 | IL1R1 | 1.84 | 1.18 |
| MOSPD3 | 1.69 | 1.51 | GREM1 | 1.82 | 1.56 |
| APRT | 1.69 | 1.16 | FGD3 | 1.82 | 2.31 |
| RAB3IL1 | 1.68 | 1.55 | ARHGDIB | 1.79 | 1.28 |
| TNFRSF1B | 1.67 | 1.46 | GATA6 | 1.78 | 1.47 |
| SLC22A5 | 1.66 | 1.22 | PRSS23 | 1.78 | 1.16 |
| ENG | 1.66 | 1.29 | KANK2 | 1.74 | 1.25 |
| SEMA3B | 1.65 | 1.50 | GADD45GIP1 | 1.70 | 1.51 |
| DBN1 | 1.65 | 1.42 | LDHA | 1.67 | 1.21 |
| IGFBP2 | 1.63 | 1.63 | TNFRSF1B | 1.67 | 1.23 |
| TBCD | 1.63 | 1.26 | ENG | 1.66 | 1.35 |
| METRN | 1.63 | 2.23 | SEMA3B | 1.65 | 1.39 |
| SERPINH1 | 1.62 | 1.21 | DBN1 | 1.65 | 1.31 |
| MBOAT7 | 1.61 | 1.44 | IGFBP2 | 1.63 | 1.34 |
| PREX1 | 1.61 | 1.25 | KIAA1549L | 1.63 | 1.44 |
| TTC9 | 1.61 | 1.65 | TBCD | 1.63 | 1.26 |
| RASAL1 | 1.60 | 1.52 | METRN | 1.63 | 1.64 |
| ZIC2 | 1.60 | 2.42 | PLLP | 1.62 | 1.46 |
| CHPF | 1.58 | 1.80 | SERPINH1 | 1.62 | 1.20 |
| PLEC | 1.58 | 1.69 | MBOAT7 | 1.61 | 1.37 |
| APLP1 | 1.58 | 1.41 | PREX1 | 1.61 | 1.28 |
| HSPG2 | 1.57 | 1.58 | RASAL1 | 1.60 | 1.45 |
| BAIAP2 | 1.55 | 1.45 | ZIC2 | 1.60 | 2.77 |
| MIF | 1.55 | 1.83 | ZNF319 | 1.59 | 1.25 |
| FZD7 | 1.55 | 1.24 | CHPF | 1.58 | 1.58 |
| MARCH4 | 1.54 | 1.28 | PLEC | 1.58 | 1.54 |
| PRSS36 | 1.52 | 1.58 | HSPG2 | 1.57 | 1.41 |
| C16orf45 | 1.51 | 1.31 | TCEAL2 | 1.56 | 1.30 |
| EXTL1 | 1.50 | 2.19 | BAIAP2 | 1.55 | 1.24 |
| | | | MIF | 1.55 | 1.72 |
| | | | FZD7 | 1.55 | 1.36 |
| | | | FADS2 | 1.54 | 1.20 |
| | | | HCK | 1.51 | 1.44 |
| | | | EXTL1 | 1.50 | 1.78 |
| | | | SLC16A3 | 1.50 | 1.65 |

### Example 7—AAT Treatment Affect the Expression of Neurodegenerative Diseases Risk Genes

Risk genes related to PD (21 genes), AD (15 genes), MS (53 genes), MCT (50 genes) PN (88 genes) and GBS (30 genes; FC) were extracted from public libraries (Timmerman, V., A. V. Strickland, and S. Zuchner. 2014., Genes (Basel), 5: 13-32; Parnell, G. P., and D. R. Booth. 2017. Front Immunol, 8: 425.; Nikolac Perkovic, M., and N. Pivac. 2019. Adv Exp Med Biol, 1192: 27-52.; Blauwendraat, C., M. A. Nalls, and A. B. Singleton. 2020. Lancet Neurol, 19: 170-78.) and their expression in AAT-treated resting microglia (FC AAT) and activated microglia (FC inflammation+AAT) was assessed. Common modified genes among AAT treatment conditions are highlighted (bold underline). Of note, the expression of several risk genes in all abovementioned diseases were modified by AAT.

TABLE 6

| Risk gene | AAT | | Risk gene | Inflammation + AAT | |
|---|---|---|---|---|---|
| PD | p-value | FC | PD | p-value | FC |
| PLA2G6 | 9.48E−03 | 1.55 | ATP13A2 | 6.94E−03 | 1.49 |
| ATP13A2 | 1.92E−02 | 1.42 | PLA2G6 | 4.87E−02 | 1.37 |
| LRP10 | 4.49E−02 | 1.29 | LRP10 | 3.15E−02 | 1.23 |
| DNAJC6 | 1.23E−02 | −1.14 | PINK1 | 4.12E−02 | 1.20 |
| GIGYF2 | 1.29E−02 | −1.16 | GBA | 4.81E−02 | 1.15 |
| SYNJ1 | 1.08E−02 | −1.33 | UCHL1 | 3.66E−02 | −1.10 |
| DNAJC13 | 3.07E−03 | −1.37 | DNAJC6 | 3.43E−02 | −1.12 |
| VPS13C | 1.84E−02 | −1.41 | SYNJ1 | 4.94E−02 | −1.23 |
| TMEM230 | 3.43E−03 | −1.42 | | | |
| VPS35 | 2.46E−03 | −1.42 | | | |
| LRRK2 | 1.98E−02 | −3.98 | | | |

TABLE 7

| Risk gene | AAT | | Risk gene | Inflammation + AAT | |
|---|---|---|---|---|---|
| AD | p-value | FC | AD | p-value | FC |
| TREM2 | 2.50E−03 | 2.22 | APOE | 3.13E−02 | 1.55 |
| ABCA7 | 3.77E−04 | 1.75 | TREM2 | 4.38E−02 | 1.47 |
| APOE | 1.05E−02 | 1.74 | BIN1 | 4.26E−03 | 1.43 |
| PSEN2 | 3.51E−03 | 1.45 | ABCA7 | 2.26E−02 | 1.36 |
| BIN1 | 3.90E−02 | 1.33 | PSEN2 | 1.00E−02 | 1.28 |
| CLU | 5.24E−03 | −1.30 | PICALM | 3.38E−02 | −1.29 |
| CD2AP | 1.25E−02 | −1.39 | | | |
| PICALM | 4.42E−03 | −1.48 | | | |

TABLE 8

| Risk gene | AAT | | Risk gene | Inflammation + AAT | |
|---|---|---|---|---|---|
| MS | p-value | FC | MS | p-value | FC |
| CYP24A1 | 8.25E−06 | 2.55 | DKKL1 | 4.05E−02 | 2.12 |
| HLA-A | 1.88E−03 | 1.48 | HLA-A | 2.42E−05 | 1.57 |
| MPV17L2 | 3.93E−02 | 1.31 | HLA-DRB1 | 1.41E−02 | 1.51 |
| AGAP2 | 3.00E−02 | 1.30 | ZMIZ1 | 8.06E−04 | 1.47 |
| CDC37 | 4.54E−02 | 1.26 | AGAP2 | 1.92E−02 | 1.45 |
| METTL1 | 3.26E−02 | 1.22 | CYP24A1 | 1.66E−02 | 1.34 |
| CD40 | 2.67E−02 | 1.17 | MPHOSPH9 | 2.92E−02 | −1.19 |
| RPS6KB1 | 4.07E−02 | −1.23 | NFKBIZ | 4.75E−02 | −1.37 |
| CBLB | 8.31E−03 | −1.24 | SLC30A7 | 3.20E−02 | −1.56 |
| PTGER4 | 3.38E−02 | −1.29 | | | |
| PTPRK | 1.93E−04 | −1.38 | | | |
| AHI1 | 6.58E−03 | −1.46 | | | |
| HLA-DRB1 | 3.40E−02 | −1.47 | | | |
| CD58 | 4.85E−02 | −1.61 | | | |
| EXTL2 | 2.52E−03 | −1.76 | | | |
| SLC30A7 | 9.06E−03 | −1.77 | | | |

TABLE 9

| Risk gene | AAT | | Risk gene | Inflammation + AAT | |
|---|---|---|---|---|---|
| MCT*/PN | p-value | FC | MCT*/PN | p-value | FC |
| SCN11A | 2.65E−02 | 4.49 | TRPV4* | 2.02E−02 | 1.72 |
| KIF1A | 1.99E−02 | 2.76 | HSPB1* | 1.06E−02 | 1.56 |
| TRPV4* | 1.14E−03 | 1.92 | TUBB3 | 2.33E−02 | 1.44 |
| HSPB1* | 2.22E−02 | 1.67 | TRPV4* | 4.70E−02 | 1.40 |
| PLEKHG5* | 4.11E−03 | 1.49 | LMNA* | 9.07E−03 | 1.39 |
| IGHMBP2* | 9.58E−03 | 1.39 | MED25* | 2.22E−02 | 1.38 |
| DNAJB2* | 9.15E−03 | 1.39 | INF2 | 4.80E−02 | 1.35 |
| LRSAM1* | 9.19E−03 | 1.36 | NDRG1* | 3.75E−03 | 1.30 |
| IFRD1 | 2.59E−02 | 1.23 | DST | 4.71E−02 | 1.30 |
| ARHGEF10 | 3.57E−03 | 1.19 | SEPT9 | 1.36E−02 | 1.29 |
| WNK1 | 4.34E−02 | −1.14 | IGHMBP2* | 3.80E−02 | 1.21 |
| CCT5 | 1.18E−02 | −1.17 | DNAJB2* | 2.76E−02 | 1.20 |
| SBF2 | 3.60E−03 | −1.20 | CCT5 | 1.91E−02 | −1.13 |
| KIF1B* | 5.00E−03 | −1.21 | KARS* | 2.35E−02 | −1.13 |
| GARS* | 1.91E−03 | −1.21 | TFG* | 1.91E−02 | −1.19 |
| GAN* | 2.14E−02 | −1.21 | GARS* | 1.12E−03 | −1.24 |
| SETX | 2.68E−02 | −1.23 | GDAP1* | 1.32E−02 | −1.24 |
| SPTLC1 | 3.73E−02 | −1.23 | SCN9A | 1.99E−03 | −1.66 |
| TFG* | 2.73E−03 | −1.28 | | | |
| DST | 2.99E−02 | −1.35 | | | |
| MTMR2* | 3.72E−03 | −1.36 | | | |
| TRIM2* | 2.09E−03 | −1.39 | | | |
| DYNC1H1* | 1.10E−02 | −1.40 | | | |
| FIG4* | 1.84E−03 | −1.42 | | | |
| SCN9A | 1.08E−02 | −1.50 | | | |
| GDAP1* | 5.03E−05 | −1.54 | | | |
| GNB4* | 6.11E−03 | −1.56 | | | |

TABLE 10

| Risk gene | | AAT | | Risk gene | | AAT | |
|---|---|---|---|---|---|---|---|
| | FC | | FC | | FC | | FC |
| GBS | GBS | p-value | AAT | GBS | GBS | p-value | AAT |
| GUK1 | −2.13 | 1.38E−02 | 1.57 | GUK1 | −2.13 | 3.31E−02 | 1.31 |
| HAGH | −2.14 | 1.07E−02 | 1.40 | NFIL3 | 2.83 | 6.38E−03 | −1.22 |
| GLRX5 | −2.48 | 4.32E−02 | 1.12 | ZNF12 | 2.79 | 4.52E−02 | −1.23 |
| HMGB2 | 3.63 | 7.91E−03 | −1.20 | FOS | 4.02 | 1.19E−02 | −1.54 |
| ZNF12 | 2.79 | 3.05E−02 | −1.26 | MARCKS | 2.81 | 2.06E−03 | −1.84 |
| CDC42 | 2.88 | 8.15E−03 | −1.35 | LY96 | 3.27 | 1.72E−02 | −2.31 |
| SMCHD1 | 2.81 | 3.33E−02 | −1.39 | — | | — | — |
| PKN2 | 2.77 | 1.66E−02 | −1.47 | | | | |
| SENP6 | 2.76 | 1.91E−02 | −1.47 | | | | |
| MARCKS | 2.81 | 5.40E−04 | −2.06 | | | | |
| FOS | 4.02 | 6.60E−04 | −2.31 | | | | |

### Example 8

Further experiments include CSF-1 treatment for (MΦ) to M1 macrophage transition optimization, Dose dependance of IFNγ for M1 macrophage activation, AAT treatment on resting and IFNγ-activated macrophage and RNA extraction and RNAseq and analysis. Whereby the cells are human primary resting (MΦ) or M1-differentiated macrophages. The treatment includes pre-treatment for 24 h with AATs, followed by cell activation (or not) with CSF-1 or IFN in presence of AAT for 24h. Cells are further treated 48h in AAT and finally tested for pro-inflammatory cytokine release (IL-6, TNFα, IL-1β, IL-8; multiplexing readout) using the culture supernatant or alternatively test for Luciferase activity (MHCII^{Luc} line) and concomitantly extract RNA for microarray analysis. Cultures with consistent readouts for the cytokine release or the luciferase assay will be used as samples for RNA extraction and microarray analysis.

### Experimental Conditions: Every Condition is Performed in Triplicate

Untreated (no AAT, no CSF-1) Resting macrophage control
IFNγ: activated macrophage control
AAT (Plasma-derived; Sigma or recombinant; Lonza): AAT effect on resting macrophage
IFNγ and AAT (Plasma-derived; Sigma or Lonza): AAT anti-inflammatory effect on activated macrophage

### Output:

Resting state macrophage gene expression
Pro-inflammatory differentially regulated genes (fold of untreated; significant p-value; fold up/down-regulation threshold)
AAT-driven gene expression change on resting and activated macrophage
Difference between recombinant and plasma-derived AAT gene regulation

### Methods

### Human Microglial Cell Line Culture

HMC3-MHCII^{Luc} cell line coding for Renilla luciferase under major histocompatibility complex II promoter (HLA-DRA) has been described as a valuable tool to study human microglial activation by and was obtained from Prof Karl-Heinz Krause, University of Geneva. It was transduced with a lentiviral vector to obtain the HMC3-MHCII^{Luc}; Ubi^{AAT} cell line (See FIGS. 3A, 3B and 3C). Both HMC3-MHCII^{Luc} and HMC3-MHCII^{Luc}; Ubi^{AAT} cell lines were cultured on TC treated cell culture dishes (CELLSTAR®, Greiner, 7.664160) in DMEM high glucose+glutamine (Gibco, 41965039) supplemented with 10% (v/v) fetal bovine serum (FBS, Gibco, 10270106) and 100 μg/ml penicillin/streptomycin (Pen/Strep, ThermoFisher, 15070063). Cultures were maintained at 37° C. in a 5% CO2 atmosphere. Passage was done by quickly rinsing the cells in PBS 1×, 3 min trypsinization at RT (Tryple Express, ThermoFisher, 12604021) followed by centrifugation (5 min, 1000 RPM) and resuspension in the abovementioned supplemented DMEM. Cells were counted and plated at desired concentration.

### Human Microglial Cell Line Transduction

The lentivirus coding for human AAT under ubiquitin promoter and GFP under human PGK promoter was obtained according to the protocol described in Marc Giry-Laterrière, Els Verhoeyen, and Patrick Salmon, 2011, Methods in molecular biology. In brief, 4.5×10⁶ HEK cells were plated in a Ø100 mm dish and transfected 16h later with 15 μg of pCWXPG-UBI-SP::AAT, 10 μg of packaging plasmid (psPAX2, gift from Didier Trono [Addgene plasmid 12260]), and 5 μg of envelope (pMD2G, gift from Didier Trono [Addgene plasmid 12259]). The medium was changed 8h post-transfection. After 48h, viral supernatant was collected and filtered using 45 μm PVDF filters and stored at −80° C. Titer of the virus was done and HMC3-MHCII^{Luc}; Ubi^{AAT} cell lines with approximately 100% and 50% of cells expressing the AAT were selected for experimental conditions.

### IFNγ-Mediated Human Microglial Activation

HMC3-MHCII^{Luc} cell line was seeded into 96-well plates at a density of approximately 2500 cells/well. 24h after, their activation was induced with a 24h-long IFNγ (Sigma, SRP3058) presentation at ranged concentrations (0.1; 1; 10 or 100 ng/ml). IFNγ was then removed and cells cultured for 48h before beeing assessed for cell viability and activation (see FIGS. 2A and 2B).

### Exogenous/Endogenous AATs Treatment on IFNγ-Activated Human Microglia

HMC3-MHCII^{Luc} and HMC3-MHCII^{Luc}; Ubi^{AAT} (endogenous AAT) cell lines were seeded into 96-well plates at a density of approximately 2500 cells/well. HMC3-MHCII^{Luc} cell line was plated and was added plasma-derived AAT and recombinant AATs (produced in CHO cells, AAT 1 and AAT 2) 3h later and at ranged concentration (1; 10 or 25 μM). 24h after, still in the presence of exogenous or endogenous AAT, microglial activation was induced with a 24h-long IFNγ presentation (10 ng/ml). IFNγ was then removed and both HMC3-MHCII^{Luc} and HMC3-MHCII^{Luc}; Ubi^{AAT} cells were cultured for 48h in exogenous or endogenous presence of AAT before cell cultures were beeing assessed for cell viability and activation (See FIGS. 3A, 3B, 3C and 4A,4B, 4C).

### Human Microglia Cell Viability and Activation Measurement

Viability (Cell Counting Kit-8, Sigma, 96992) and activation (Renilla-Glo® Luciferase Assay System, Promega, E2710) of HMC3-MHCII^{Luc} and HMC3-MHCII^{Luc}; Ubi^{AAT} cell cultures were measured according to the manufacturers' protocols.

### RNA Collection, Sequencing and Differential Expression Analysis

RNA extraction was achieved with RNeasy Mini kit (Qiagen) according to manufacturer's protocol. RNA samples from plasma-derived AAT and recombinant AAT Nr. 2 were checked for quality (2100 Bioanalyzer, Agilent) and libraries prepared with Truseq RNA Library Kit (Illumina, RS-122-2001). Libraries were sequenced (HiSeq 4000, Illumina) controlled for the quality of sequencing (FastQC), mapped on the human genome (STAR v.2.7.0f; UCSC hg38), reads were counted (HTSeq v0.9.1) and the differential expression analysis was performed with the R/Bioconductor package (edgeR 1.30.1.).

### RNA Collection, Sequencing and Differential Expression Analysis

Human monocyte-derived M1 macrophage (GM-CSF, PromoCell, C-12916) were cultured on fibronectin-coated cell culture dishes in M1-Macrophage Generation Medium XF and activated with CSF-1 (50 ng/ml, Sigma, SRP3058) according to the manufacturer's protocol, Cultures were maintained at 37° C. in a 5% CO2 atmosphere.

### Cytokine Multiplex Assay

Cell culture supernatant was collected and measure for IL-6, TNFα, IL-1β and IL-8 with bead based Luminex assay according to the manufacturer's protocol.

### Cell Free TACE/ADAM17 Activity

TACE activity and its inhibition by human AAT (AAT) was performed with Recombinant Human TACE/ADAM17 kit (930-ADB and ES003, R&D Systems) in black 96-well immuno plates (437111, ThermoFisher Scientific). The enzymatic activity of TACE/ADAM17 was measured by mixing 0.005 μg of rhTACE with 10 μM of Mca-PLAQAV-Dpa-RSSSR-NH2 fluorogenic peptide substrate III in assay buffer (25 mM Tris, 2.5 μM ZnCl2, 0.005% Brij-35 (w/v), pH 9.0) to a final volume of 100 μl. AAT (Sigma Aldrich, batch A6150) was resuspended in water (vehicle), control TACE/ADAM17 activity was assessed in presence of the vehicle (amount used for AAT 100 μM). AAT was added at different concentration (0, 6.25, 12.5, 25, 50 and 100 μM) to assess its dose-dependent inhibition of TACE/ADAM17. All conditions were performed in triplicates. Activity was measured as relative fluorescent unit (RFU) in a kinetic mode (9 time points over 5 min) with a SpectraMax iD3 Microplate Reader (low PMT gain, is exposition, top read at 1 mm, wavelength: excitation 320 nm, emission 405 nm). Bar graph as percentage of control activity was obtained by averaging the values obtained over the 5 min for each of the condition.

### Animals

As a murine model of CMT1A we used C3-PMP22 transgenic mice (B6.Cg-Tg(PMP22)C3Fbas/J, The Jackson Laboratory) which express three copies of a wild-type human peripheral myelin protein 22 (PMP22) gene (Verhamme, Camiel, et al. Journal of Neuropathology & Experimental Neurology 70.5 (2011): 386-398). Mice were housed in macrolon cages with filter hoods, in a continuously air-filtered room, thereby avoiding contamination. During experiments, paired animals were caged at a constant temperature with a day/night cycle of 12/12 hours. Animals were fed (control tap water and nutrition) ad libitum. Animal protocol is approved by the Animal Studies Committee of Languedoc Roussillon. This protocol and the laboratory procedures comply with French legislation, which implements the European Directives (Reference Number: D3417223, APAFIS #23920-2020020320279696 v3). Animal health was followed on a daily basis to ensure that only animals in good health enter the testing procedures and follow up the study.

### In Vivo Study Paradigm

Animals were split in 3 groups (wild-type control (subcutaneous 0.9% NaCl), CMT1A-vehicle (subcutaneous 0.9% NaCl), CMT1A-human alpha-1 antitrypsin (subcutaneous, twice daily, 50 mg/kg per injection)) of 3 mice each (3 weeks old males of 18±2.5 g at the beginning of study) and all went through the following protocol after 7 days of acclimation on site.

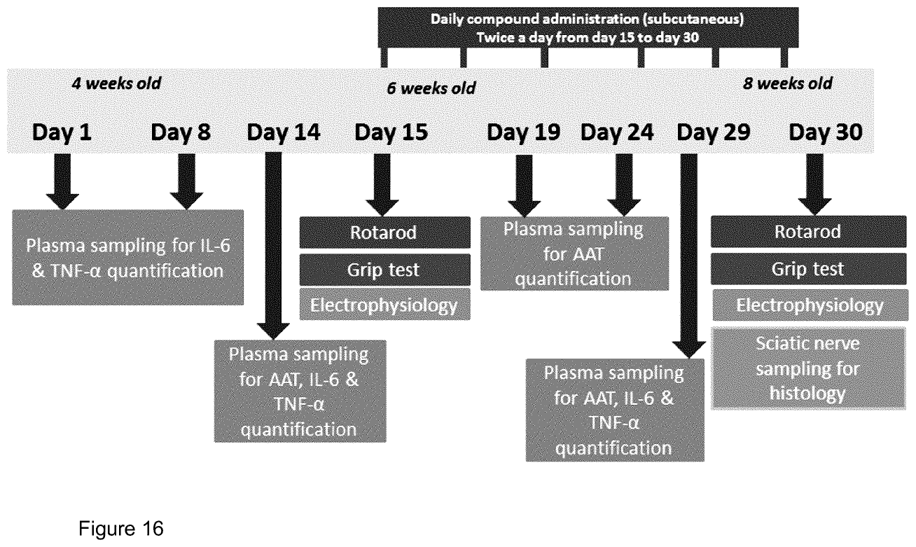

Starting from the age of 4 weeks, animals were subjected to blood samplings for the determination of interleukin-6 (IL-6) and tumor necrosing factor alpha (TNFα) levels, as described in FIG. 16.

Plasma levels of AAT were evaluated every 5 days from the first day of treatment to the last treatment day.

On the first and last day of treatment, the neuromuscular performance of animals was tested with a rotarod test, grip test and sciatic nerve electrophysiology test. After the last treatment at 8 weeks, these tests were repeated, and animals were sacrificed and the left sciatic nerve was sampled for histological evaluation of the number and size of neurons.

### Example 9

TACE activity is assessed according to the manufacturer's instruction (Recombinant Human TACE/ADAM17 kit, 930-ADB, R&D Systems) in kinetic mode, without or with different AAT concentration. All conditions were done in triplicates and are shown as mean±SD (FIGS. 5A, 5B and 5C).

### Example 10

The most common type of CMT is CMT1A, characterized by a duplication of the PMP22 gene leading to an accumulation of the pmp22 protein in the Schwann cell and progressive demyelination. PMP22 is a tetraspan glycoprotein contained in compact myelin of the peripheral nervous system. Duplication of PMP22 has been associated with the onset of Charcot-Marie-Tooth disease type 1A (CMT1A). The C3-PMP22 transgenic mice (B6.Cg-Tg(PMP22)C3Fbas/J) express three copies of a wildtype human peripheral myelin protein 22 (PMP22) gene. The cause and effect between the additional PMP22 gene and CMT1A are still not well understood and remain elusive to this day. Several plausible hypotheses are, nevertheless, available to link the genetic abnormality, that is the duplication of the PMP22 gene, to the pathology's manifestation. Without being bound to theory, PMP22 overexpression may exert a negative effect on the formation of myelin sheaths in the peripheral nervous system (PNS). These mice present an age-dependent demyelinating neuropathy characterized by predominantly distal loss of strength and sensation. C3-PMP mice show no overt clinical signs at 3 weeks and develop progressive and observable neuromuscular impairment after 4 weeks. The mice have stable, low nerve conduction velocities the same way as in adults with human CMT1A. Myelination is delayed in these mice, and they contain reduced numbers of myelinated axons at 3 weeks of age. This mouse model was used to study the effect of AAT in different paradigms.

Positive efficacy of AAT administration was observed after two weeks in the CMT1A mice by increasing rotarod latency, grip strength and nerve conduction performances compared to an untreated control group. Moreover, there is no observable body weight loss in the AAT treated group compared to the vehicle group suggesting the absence of systemic toxicology of the compound at these experimental conditions (Table 11).

TABLE 11
Body weight
WT control

| | 4 weeks | 5 weeks | 6 weeks | 7 weeks | 8 weeks |
|---|---|---|---|---|---|
| | old | old | old | old | old |
| Mouse 1.1 | 18.6 | 19.5 | 19.8 | 20.2 | 21.1 |
| Mouse 1.2 | 19.1 | 19.7 | 19.8 | 20.3 | 20.9 |
| Mouse 1.3 | 18.4 | 18.9 | 19.4 | 19.8 | 20.6 |
| MEAN | 18.70 | 19.37 | 19.66 | 20.12 | 20.90 |
| SD | 0.36 | 0.40 | 0.20 | 0.25 | 0.25 |
| SEM | 0.21 | 0.23 | 0.12 | 0.14 | 0.14 |

CMT1A + vehicle

| | 6 weeks | 8 weeks | 6 weeks | 8 weeks | 6 weeks |
|---|---|---|---|---|---|
| | old | old | old | old | old |
| Mouse 2.1 | 19.5 | 20.4 | 20.8 | 21.3 | 21.4 |
| Mouse 2.2 | 19.2 | 19.8 | 20.4 | 20.6 | 21.2 |
| Mouse 2.3 | 18.1 | 18.4 | 19.0 | 19.7 | 20.5 |
| MEAN | 18.93 | 19.57 | 20.08 | 20.51 | 21.05 |
| SD | 0.74 | 1.03 | 0.98 | 0.79 | 0.49 |
| SEM | 0.43 | 0.60 | 0.57 | 0.46 | 0.29 |

CMT1A + hAAT

| | 6 weeks | 8 weeks | 6 weeks | 8 weeks | 6 weeks |
|---|---|---|---|---|---|
| | old | old | old | old | old |
| Mouse 3.1 | 18.7 | 19.2 | 19.6 | 20.1 | 20.8 |
| Mouse 3.2 | 19.2 | 20.1 | 20.5 | 21.1 | 21.8 |
| Mouse 3.3 | 18.6 | 18.6 | 19.1 | 19.9 | 20.6 |
| MEAN | 18.83 | 19.31 | 19.73 | 20.33 | 21.06 |
| SD | 0.32 | 0.78 | 0.74 | 0.63 | 0.62 |
| SEM | 0.19 | 0.45 | 0.43 | 0.36 | 0.36 |

Sciatic nerve electrophysiology (EMG) provides sensitive and quantitative approach to measure compound muscle action potential and nerve conduction velocity amplitude in the animals and was done by stimulation of the sciatic nerve. Similar compound muscle action potential (CMAP) amplitudes were observed between groups at the baseline (6 weeks old). As expected, a strong and significant decrease of CMAP amplitude was observed in the CMT1A+vehicle group compared to the wild type control group at 8 weeks old. Results show improvement of the EMG parameters for CMT1A mice treated with AAT compared to the control (FIG. 6A, 6B and Table 12, 13, 14) suggesting a positive efficacy of AAT on axonal degeneration induced by CMT1A disorder.

Lower nerve conduction velocities (NCV) were observed in both CMT1A groups compared to the wild type control group at the baseline (6 weeks old). At the baseline, the differences of NCVs between groups were not statistically significant. As expected, a strong and significant decrease of NCV was observed in the CMT1A+vehicle group compared to the wild type control group at 8 weeks old. The CMT1A+AAT treated group presented an increase of the NCV compared to the vehicle treated group. Because the nerve conduction velocity depends on the myelin sheath integrity, these data also suggest a positive efficacy of AAT on the Schwann cell demyelination induced by CMT disorder.

TABLE 12
Sciatic nerve electrophysiology

| Amplitude (mV) | | T1 distal (s) | | T2 proximal (s) | | Conduction velocity (m/s) | |
|---|---|---|---|---|---|---|---|
| 6 weeks old | 8 weeks old | 6 weeks old | 8 weeks old | 6 weeks old | 8 weeks old | 6 weeks old | 8 weeks old |

WT control

| | | | | | | | | |
|---|---|---|---|---|---|---|---|---|
| Mouse 1, 1 | 8.881 | 7.462 | 0.00251 | 0.00456 | 0.00298 | 0.00508 | 31.501 | 28.846 |
| Mouse 1, 2 | 6.982 | 6.204 | 0.00331 | 0.00426 | 0.00376 | 0.00468 | 33.523 | 35.937 |
| Mouse 1, 3 | 7.140 | 8.721 | 0.00271 | 0.00739 | 0.00319 | 0.00795 | 31.071 | 26.986 |
| MEAN | 7.668 | 7.462 | | | | | 32.032 | 30.590 |
| SD | 1.054 | 1.259 | | | | | 1.309 | 4.723 |
| SEM | 0.608 | 0.727 | | | | | 0.756 | 2.727 |

CMT1A + vehicle

| | | | | | | | | |
|---|---|---|---|---|---|---|---|---|
| Mouse 2, 1 | 6.942 | 1.785 | 0.00228 | 0.00215 | 0.00292 | 0.00354 | 23.644 | 10.720 |
| Mouse 2, 2 | 6.452 | 3.162 | 0.00293 | 0.00235 | 0.00357 | 0.00379 | 23.593 | 10.392 |
| Mouse 2, 3 | 5.835 | 3.735 | 0.00137 | 0.00213 | 0.00191 | 0.00302 | 27.863 | 16.923 |
| MEAN | 6.410 | 2.894 | | | | | 25.034 | 12.678 |
| SD | 0.555 | 1.002 | | | | | 2.451 | 3.680 |
| SEM | 0.320 | 0.579 | | | | | 1.415 | 2.124 |

CMT1A + hAAT

| | | | | | | | | |
|---|---|---|---|---|---|---|---|---|
| Mouse 3, 1 | 7.030 | 3.799 | 0.00202 | 0.00251 | 0.00250 | 0.00313 | 31.277 | 23.904 |
| Mouse 3, 2 | 6.025 | 5.260 | 0.00207 | 0.00236 | 0.00295 | 0.00318 | 17.182 | 18.310 |
| Mouse 3, 3 | 6.359 | 7.822 | 0.00160 | 0.00228 | 0.00217 | 0.00302 | 26.268 | 20.464 |
| MEAN | 6.471 | 5.627 | | | | | 24.909 | 20.892 |
| SD | 0.511 | 2.036 | | | | | 7.145 | 2.821 |
| SEM | 0.295 | 1.176 | | | | | 4.125 | 1.629 |

TABLE 13
Mean compound muscle action potential
Compound muscle action potential, mV (Mean ± SEM)

| | 6 weeks old | 8 weeks old |
|---|---|---|
| WT control | 7.67 ± 0.61 | 7.46 ± 0.73 |
| CMT1A + vehicle | 6.41 ± 0.32 | 2.89 ± 0.58*** |
| CMT1A + AAT | 6.47 ± 0.30 | 5.63 ± 1.18^{†} |

2-way ANOVA with repeated measures and Bonferroni t-test
***p < 0.001 vs WT control;
^{†}p < 0.05 vs CMT1A + vehicle

TABLE 14
Mean nerve conduction velocity
Nerve conduction velocity, m/s (Mean ± SEM)

| | 6 weeks old | 8 weeks old |
|---|---|---|
| WT control | 32.03 ± 0.76 | 30.59 ± 2.73 |
| CMT1A + vehicle | 25.03 ± 1.42 | 12.68 ± 2.12*** |
| CMT1A + AAT | 24.91 ± 4.13 | 20.89 ± 1.63* |

2-way ANOVA with repeated measures and Bonferroni t-test
*; ***p < 0.01; p < 0.001 vs WT control

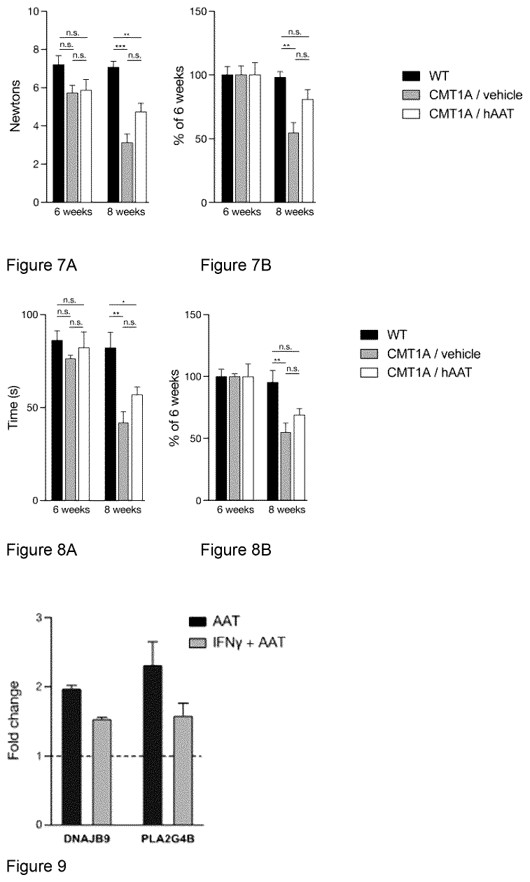

Grip strength test measures neuromuscular strength by assessing the animal's grasp of a metal grid. Lower grip strength was observed in the CMT groups compared to the wild type control group at the baseline (6 weeks old). At the baseline, the differences of grip strengths between groups were not statistically significant. As expected, a strong and significant decrease of grip strength was observed in the CMT1A+vehicle group compared to the wild type control group at 8 weeks old. Results show improvement of the grip strength for CMT1A mice treated with AAT compared to the control group (FIG. 7A, 7B and Table 15, 16).

TABLE 15
Grip strength

| | |
|---|---|
| Animal | Grip strenght at 6 weeks old (baseline) (Newtons) |

| | | | | |
|---|---|---|---|---|
| number | set 1 | set 2 | set 3 | Mean/animal |

WT control

| | | | | |
|---|---|---|---|---|
| 1.1 | 5.45 | 6.96 | 8.64 | 7.02 |
| 1.2 | 3.51 | 6.73 | 9.20 | 6.48 |
| 1.3 | 8.84 | 6.86 | 8.61 | 8.10 |

CMT1A + vehicle

| | | | | |
|---|---|---|---|---|
| 2.1 | 1.71 | 6.57 | 7.76 | 5.35 |
| 2.2 | 4.74 | 5.76 | 5.29 | 5.26 |
| 2.3 | 6.90 | 5.09 | 7.59 | 6.53 |

CMT1A + AAT

| | | | | |
|---|---|---|---|---|
| 3.1 | 5.84 | 7.62 | 1.82 | 5.10 |
| 3.2 | 7.10 | 7.19 | 6.62 | 6.97 |
| 3.3 | 5.94 | 3.08 | 7.52 | 5.51 |

| | |
|---|---|
| Animal | Grip strenght at 8 weeks old (Newtons) |

| | | | | |
|---|---|---|---|---|
| number | set 1 | set 2 | set 3 | Mean/animal |

WT control

| | | | | |
|---|---|---|---|---|
| 1.1 | 6.31 | 4.90 | 8.83 | 6.68 |
| 1.2 | 5.46 | 7.51 | 7.41 | 6.79 |
| 1.3 | 6.46 | 9.38 | 7.29 | 7.71 |

CMT1A + vehicle

| | | | | |
|---|---|---|---|---|
| 2.1 | 4.29 | 4.76 | 2.90 | 3.98 |
| 2.2 | 5.58 | 1.27 | 2.05 | 2.96 |
| 2.3 | 0.61 | 3.43 | 3.16 | 2.40 |

CMT1A + AAT

| | | | | |
|---|---|---|---|---|
| 3.1 | 6.55 | 4.38 | 5.43 | 5.45 |
| 3.2 | 4.01 | 6.93 | 3.63 | 4.86 |
| 3.3 | 2.76 | 3.67 | 5.21 | 3.88 |

TABLE 16
mean grip strength
Grip strength, newtons (Mean ± SEM)

| | 6 weeks old | 8 weeks old |
|---|---|---|
| WT control | 7.20 ± 0.48 | 7.06 ± 0.33 |
| CMT1A + vehicle | 5.71 ± 0.41 | 3.12 ± 0.46*** |
| CMT1A + AAT | 5.86 ± 0.57 | 4.73 ± 0.46** |

2-way ANOVA with repeated measures and Bonferroni t-test
**; ***p < 0.01; p < 0.001 vs WT control

The rotarod test measures neuromuscular coordination by assessing the capacity of the animals to stay in balance on a rotating cylinder. Similar rotarod latency was observed between groups at the baseline (6 weeks old). As expected, a strong and significant decrease of rotarod latency was observed in the CMT1A+vehicle group compared to the wild type control group at 8 weeks old. Results show improvement of the rotarod latency for CMT1A mice treated with AAT compared to the control group (FIG. 8A, 8B and Table 17,18).

TABLE 17
Rotarod latency

| Animal | Rotarod latency at 6 weeks old (baseline) (seconds) | | | |
|---|---|---|---|---|
| number | set 1 | set 2 | set 3 | Mean/animal |
| | WT control | | | |
| 1.1 | 64.7 | 94.3 | 88.4 | 82.46 |
| 1.2 | 105.8 | 98.8 | 84.9 | 96.49 |
| 1.3 | 77.2 | 115.0 | 46.3 | 79.50 |
| | CMT1A + vehicle | | | |
| 2.1 | 69.5 | 82.5 | 65.8 | 72.62 |
| 2.2 | 56.6 | 77.8 | 101.8 | 78.77 |
| 2.3 | 61.2 | 72.3 | 84.3 | 72.62 |
| | CMT1A + AAT | | | |
| 3.1 | 85.0 | 102.6 | 65.5 | 84.36 |
| 3.2 | 51.4 | 93.4 | 68.8 | 71.19 |
| 3.3 | 59.6 | 92.1 | 67.1 | 72.91 |

| | |
|---|---|
| Animal | Rotarod latency at 8 weeks old (seconds) |

| | | | | |
|---|---|---|---|---|
| number | set 1 | set 2 | set 3 | Mean/animal |

WT control

| | | | | |
|---|---|---|---|---|
| 1.1 | 61.9 | 106.2 | 84.0 | 84.03 |
| 1.2 | 60.0 | 72.9 | 67.4 | 66.77 |
| 1.3 | 110.8 | 70.2 | 106.2 | 95.69 |

CMT1A + vehicle

| | | | | |
|---|---|---|---|---|
| 2.1 | 30.6 | 78.2 | 51.0 | 53.26 |
| 2.2 | 40.8 | 51.0 | 26.3 | 39.36 |
| 2.3 | 35.7 | 38.3 | 24.9 | 32.95 |

CMT1A + AAT

| | | | | |
|---|---|---|---|---|
| 3.1 | 62.1 | 35.7 | 51.5 | 49.76 |
| 3.2 | 83.2 | 39.5 | 71.3 | 64.65 |
| 3.3 | 46.0 | 46.0 | 76.4 | 56.12 |

TABLE 18
mean rotarod latency
Rotarod latency, seconds (Mean ± SEM)

| | 6 weeks old | 8 weeks old |
|---|---|---|
| WT control | 86.15 ± 5.24 | 82.16 ± 8.40 |
| CMT1A + vehicle | 74.67 ± 2.05 | 41.86 ± 5.99*** |
| CMT1A + AAT | 76.15 ± 4.13 | 56.84 ± 4.31* |

2-way ANOVA with repeated measures and Bonferroni t-test
*; ***p < 0.05; p < 0.001 vs WT control

### Example 11

It appears that PMP22 protein is particularly important in protecting nerves from physical pressure, helping them restore their structure after being pinched or squeezed (compressed). Compression can interrupt nerve signaling, leading to the sensation commonly referred to as a limb “falling asleep.” The ability of nerves to recover from normal, day-to-day compression, for example when sitting for long periods, keeps the limbs from constantly losing sensation. In CMT1A patients the myelination process is not properly complete, and the pathological symptoms associated with the disease become apparent most often after the second decade of life.

The PMP22 gene also plays a role in the growth of Schwann cells and the process by which cells mature to carry out specific functions (differentiation). Before they become part of myelin, newly produced PMP22 proteins are processed and packaged in specialized cell structures called the endoplasmic reticulum and the Golgi apparatus. Completion of these processing and packaging steps is critical for proper myelin function. CMT1A's pathomechanics is characterized by the absence of myelin sheaths due to an extra PMP22 gene, which is responsible for the abnormally high concentration of the peripheral myelin protein 22 (PMP22) in Schwann cells. GSEA analysis done on human microglial cells, AAT treatment has shown upregulation of genes related to the unfolded protein response (UPR) pathway and cell-survival (anti-apoptotic (FIG. 4C, FIG. 9).

### Example 12

ADAM17, also known as TACE, is a transmembrane protein that includes an extracellular zinc-dependent protease domain. In the context of CMT1A, ADAM17 is known for its inhibitory effect on SCs mediated myelination through neuregulin 1 type III (NRG1-III). It is postulated that AAT was able to cross the blood nerve barrier (BNB) and interact with ADAM17 to successfully inhibit its activity and by doing so allowing SCs to “manually” overcome the distress signal that an overloaded ER with PMP22 generates and facilitating the formation of myelin sheaths around axons.

### Example 13 Plasma AAT Levels

AAT was not detected in plasma of the wild type control and CMT1A mice treated with vehicle at the analyzed time points (day 14, day 19, day 24 and day 29). The AAT was detected in plasma of CMT1A+AAT group at a mean of 6.07 μg/mL, 6.99 μg/mL, 8.14 μg/mL and 5.22 μg/mL at day 14, day 19, day 24 and day 29 respectively.

### Example 14A Sciatic Nerve Histology

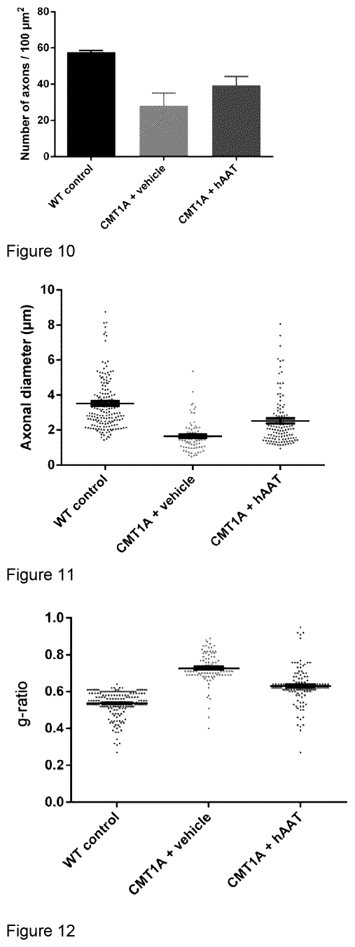

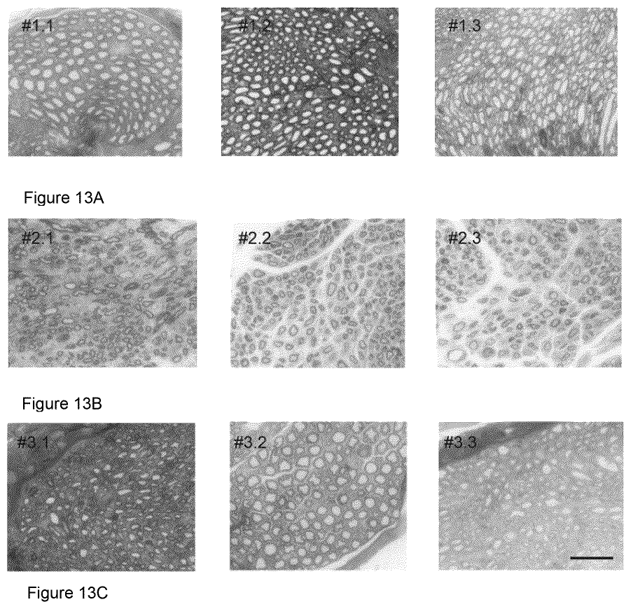

As expected a decrease of the total number of axons per surface, the axonal diameter and a significant increase of the g-ratio was observed in the CMT1A+vehicle group compared to the wild type control group at 8 weeks old (Table 19, FIG. 10 to FIG. 13C).

Slight increase of the total number of axons per surface was observed in the CMT1A+AAT treated group compared to the vehicle group. Moreover, significant increase of the axonal diameter and decrease of the g-ratio (equal to the ratio of the inner-to-outer diameter of a myelinated axon) were observed in the CMT1A animals treated with AAT compared to the vehicle treated group. Even if, the CMT1A+AAT animals also presented a number of axons, axonal diameter and g-ration statistically different than the wild type control group (Table 19, FIGS. 10 to 13C). Taken together these data suggest a positive but partial efficacy of AAT on the histopathology induced by the CMT1A disorder when administrated at 50 mg/kg twice daily by subcutaneous route.

TABLE 19
Sciatic nerve histology
Sciatic nerve histology (Mean ± SEM)

| | Axons/100 μm² | Axonal diameter (μm) | g-ratio |
|---|---|---|---|
| WT control | 57.67 ± 0.88 | 3.52 ± 0.13 | 0.54 ± 0.005 |
| CMT1A + vehicle | 28.00 ± 7.02* | 1.65 ± 0.09*** | 0.73 ± 0.010*** |
| CMT1A + AAT | 39.33 ± 4.81 | 2.53 ± 0.15*** ^{†††} | 0.63 ± 0.009*** ^{†††} |

### Example 14B—IL-6

Similar plasma IL-6 concentrations were observed between groups at the baseline (dayl) and day 8.

As expected, significant increase of plasma IL-6 concentration was observed in the CMT+vehicle group at day 14 and day 29. The terms CMT and CMT1A are used interchangeably in the example section.

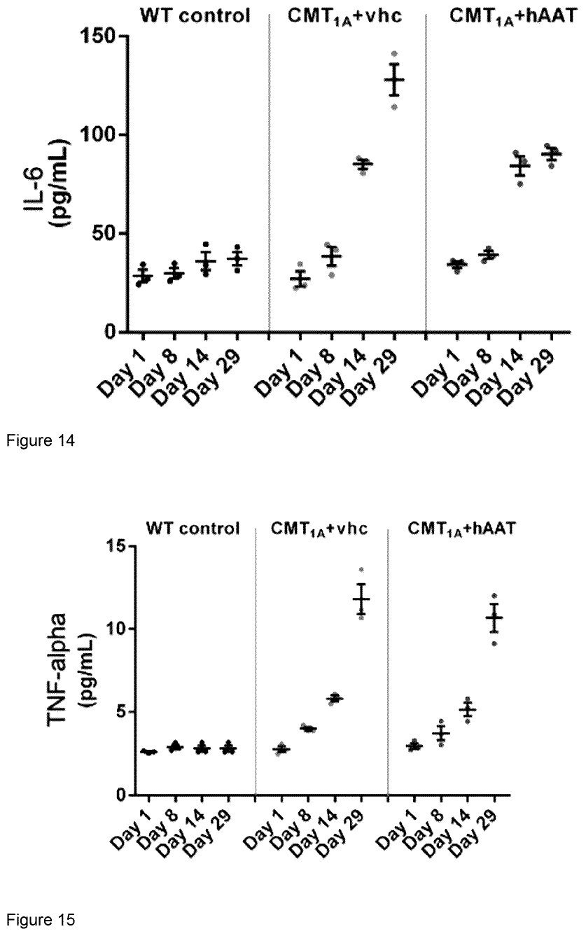

The CMT+AAT treated group also presented a significant increase of plasma IL-6 concentration compared to the baseline concentration. However, the plasma IL-6 concentration of animals treated with AAT was lower than animals treated with vehicle at day 29 (Table 20 and FIG. 14) suggesting a direct or indirect effect of AAT on this inflammatory cytokine.

TABLE 20
Mean plasma IL-6 concentration of CMT mice treated with and without AAT
Plasma IL-6 concentration, pg/mL (Mean ± SD)

| | Day 1 | Day 8 | Day 14 | Day 29 |
|---|---|---|---|---|
| WT control | 28.50 ± 3.04 | 29.74 ± 2.67 | 35.90 ± 4.516 | 37.20 ± 3.391 |
| CMT1A + vehicle | 26.90 ± 3.86 | 38.32 ± 4.78 | 85.04 ± 2.296*** | 127.84 ± 7.810*** |
| CMT1A + AAT | 34.19 ± 1.67 | 39.10 ± 1.91* | 84.21 ± 4.727** | 90.20 ± 3.085*** ^{†} |

Student t-test
*; **; ***p < 0.05; p < 0.01; p < 0.001 vs WT control at this timepoint;
^{†} p < 0.05 vs CMT1A + vehicle at this timepoint

### Example 15—TNFα

A significant increase of plasma TNFα concentration was observed in the CMT1A+vehicle group at day 14 and day 29.

The CMT1A+AAT treated group also presented a significant increase of plasma TNFα concentration at day 14 and day 29 compared to the baseline concentration (Table 21 and FIG. 15) suggesting that AAT has no effect on the levels of this inflammatory cytokine in this model of CMT1A.

TABLE 21
Plasma TNFα levels
Plasma TNFα concentration, pg/mL (Mean ± SEM)

| | Day 1 | Day 8 | Day 14 | Day 29 |
|---|---|---|---|---|
| WT control | 2.60 ± 0.05 | 2.89 ± 0.14 | 2.80 ± 0.187 | 2.80 ± 0.187 |
| CMT1A + vehicle | 2.76 ± 0.17 | 3.99 ± 0.11** | 5.80 ± 0.175*** | 11.81 ± 0.897*** |
| CMT1A + AAT | 2.94 ± 0.17 | 3.70 ± 0.41 | 5.15 ± 0.393** | 10.67 ± 0.833*** |

Student t-test
**; ***p < 0.01; p < 0.001 vs WT control at this timepoint.

### Example 16

AAT's effect on SH-SY5Y treated with 6-OHDA was evaluated by cell morphology and cell proliferation followed by cell viability quantification.

### Cells and Treatments

SH-SY5Y cells, which are commonly used to model neurodegenerative disorders, were used to produce an in vitro Parkinson's model (Que R, et al., 2021, Front. Immunol. 12). Cells were cultured in DMEMF12/Glutamax supplemented with 10% FBS.

Cells were treated for 24 hours with the neurotoxin 6-hydroxydopamine (6-OHDA, Sigma Aldrich 162957) at 50, 100 μM concentrations alone or in combination with AAT 25 μM (AAT plasma derived, Sigma Aldrich batch A9024). Cells treated with AAT alone or in combination with 6-OHDA for 24 hours, were then incubated with fresh AAT for additional 24 hours. Control cells were treated with PBS.

### Cell Growth and Viability Assay

At day 0 cells were plated in equally number in 24-well plate, the day after they were treated as described above, and final total cell count was performed at day 4 of culture (cell growth). Cell viability was assessed with Cell Counting Kit-8 (CCK-8; Sigma Aldrich 96992). After treatments, cells were incubated with 10 μl of CCK-8 solution for 2 hours in the incubator. Absorbance was measured at 450 nm using SpectraMax iD3 Microplate Reader. Experiments were performed in triplicate.

### Human IL-6 Immunoassay

Human 11-6 immunoassay (R&D D6050) was performed on cells supernatant. Briefly, 40000 cells were plated in 24-well plate and treated the day after as described in paragraph “Cells and treatment”. At the end of the treatment, cells were washed with PBS and incubated with 2% FBS medium for 24 hours, then cell culture supernatants were collected and centrifuged to remove particulates. Assay procedure was performed as described by manufactured instructions, on standard and samples duplicates. Absorbance was measured at 450 nm using SpectraMax iD3 Microplate Reader. A standard curve was prepared from seven IL-6 standard dilutions and IL-6 sample concentrations determined.

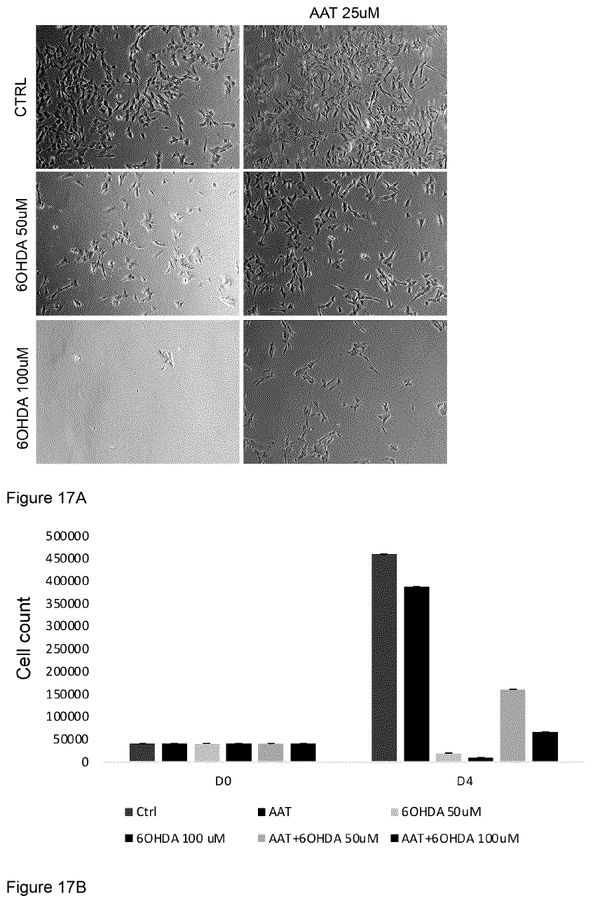

6-OHDA at 50-100 μM for 24 hours induced cells proliferation impairment by inducing cell death and reducing the number of cells after 4 days of culture (FIGS. 17A and 17B). In contrast combined treatment with AAT significantly increased the number of cells counted compared to 6-OHDA alone (FIGS. 17A and 17B), indicating a positive effect of AAT on cell survival/proliferation. T-test p-values are significative for: ctrl vs 6ohda p=0.001; 6ohda vs AAT+6ohda p=0.003. Ctrl vs AAT is not significant. Error bars are S.E.M.

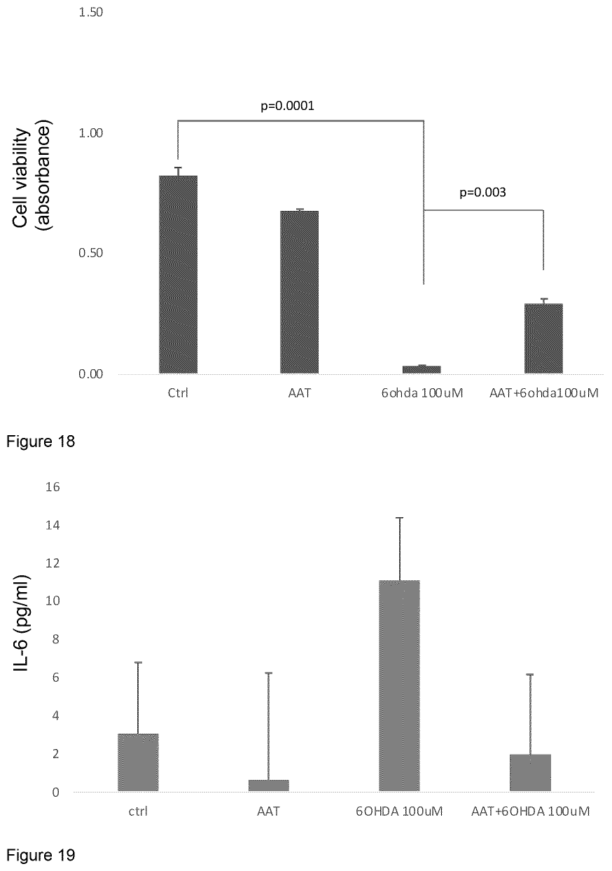

Treated cells were then challenged in a cell viability assay. SH-SY5Y treated with 6-OHDA alone showed strong reduction of cell viability compared to control cells and cells treated with AAT only (FIG. 18).

Cell count as well as the viability of the cells were significantly enhanced when 6-OHDA treatment was combined with AAT, compared to 6-OHDA treatment alone (FIG. 18). P-values are calculated with t-test, non-significant differences were observed between control and AAT alone. All conditions were performed in triplicates. Based on these results we can conclude that AAT administration in a PD cell model (SH-SY5Y induced by 6-OHDA) has favorable effect on cell growth and viability, possibly protecting cells from 6-OHDA induced death.

In addition, to explore the role of AAT on pro-inflammatory cytokines we performed IL-6 quantification in cells supernatant. Medium from cells induced by 6-OHDA had increased level of IL-6 compared to control medium, on the contrary the medium collected from cells treated with AAT displayed less concentration of IL-6 (FIG. 19). T-test p-value are significant for ctrl vs 6-OHDA p=0.05 and not significant for the other comparison.

### Example 17

C57BL/6 mice were received at 8 weeks of age and housed under controlled conditions for the duration of the study. Three groups of 4 animals each were assigned to the following subcutaneous treatments twice daily at 7:00 AM and 7:00 PM for 2 weeks, from 20 to 35 days after the start of disease induction:
Wild type (WT) mice receiving the vehicle, 0.9% NaCl (positive control)
CIDP mice receiving the vehicle, 0.9% NaCl (negative control)
CIDP mice receiving human AAT (AAT), 50 mg/kg per injection

Disease induction consisted in subcutaneous injection of mouse sciatic nerve homogenate in phosphate buffer saline (PBS)+Freund's adjuvant mixture at 10 mL/kg. For each treatment, 3 different body sites injections were performed. Control mice also received three subcutaneous injections of PBS+Freund's adjuvant at the same 3 different body sites.

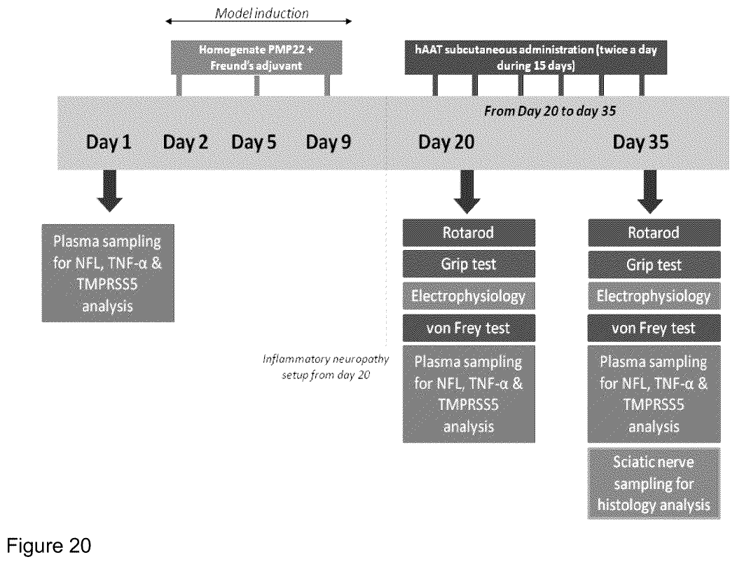

On the day preceding disease induction (Day 1), on the first (Day 20) and on the last (Day 35) treatment day, animals were subjected to blood samplings for the determination of neurofilament light chain (NfL), tumour necrosing factor alpha (TNFα) and TMPRSS5 levels, as described in FIG. 20. In addition, plasma levels of AAT were evaluated on the last treatment day.

On the first (Day 20) and last (Day 35) day of treatment, the neuromuscular and performance of animals was tested with a rotarod test, grip test and a sensory test using the Von Frey filament test, as well as sciatic nerve electrophysiology test. After the last treatment at Day 35, these tests were repeated, animals were sacrificed and the left sciatic nerve was sampled for histological evaluation of the number and size of neurons.

### Rotarod Test

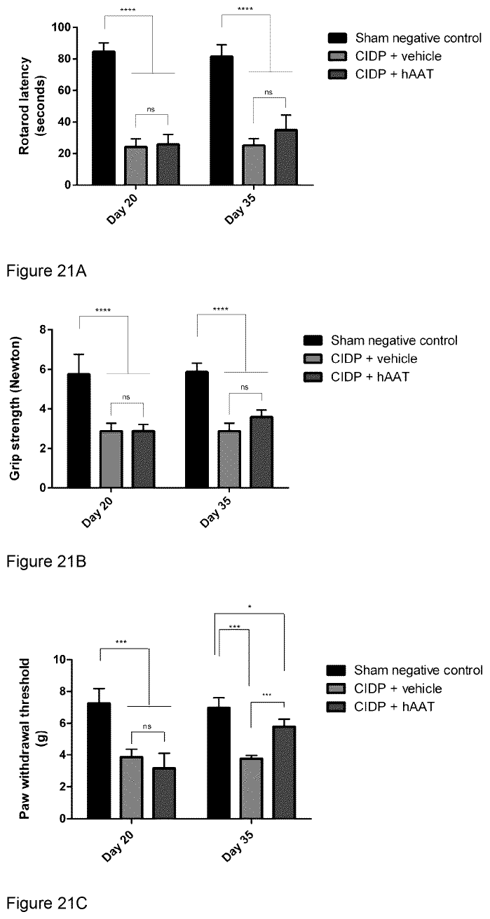

A strong decrease of rotarod latency was observed in the CIDP groups at Day 20, compared to the control group. On Day 35, the CIDP+AAT treated group presented an increase of rotarod latency compared to the vehicle treated group (Table 22, FIG. 21 A).

TABLE 22
Mean rotarod latency
Rotarod latency, seconds (Mean ± SEM)

| | Day 20 | Day 35 |
|---|---|---|
| WT control | 84.65 ± 5.55 | 81.50 ± 7.44 |
| CIDP + vehicle | 24.21 ± 5.16 | 25.17 ± 4.39 |
| CIDP + AAT | 25.96 ± 6.16 | 34.96 ± 9.47 |

### Grip Strength Test

Lower grip strength was observed in the CIDP groups compared to the wild type control group at the baseline (Day 20). On Day 35, the CIDP+AAT treated group presented an increase of grip strength compared to the vehicle treated group (Table 23 and 21 B).

TABLE 23
Mean grip strength
Grip strength, newtons (Mean ± SD)

| | Day 20 | Day 35 |
|---|---|---|
| WT control | 5.77 ± 0.99 | 5.88 ± 0.43 |
| CIDP + vehicle | 2.88 ± 0.40 | 2.72 ± 0.20 |
| CIDP + AAT | 2.89 ± 0.32 | 3.59 ± 0.35 |

### Von Frey Test

Lower paw withdrawal threshold was observed in the CIDP groups compared to the wild type control group at the baseline (Day 20). On Day 35, the CIDP+AAT treated group presented an increased threshold of paw withdrawal compared to the vehicle treated group (Table 24 and FIG. 21 C). The results from this test suggest that AAT presents a statistically significant positive effect on the pain threshold (alleviating pain) in these CIDP mice when administrated with AAT twice a day at 50 mg/kg by the subcutaneous route.

TABLE 24
Mean paw withdrawal threshold
Von Frey, paw withdrawal threshold, grams (Mean ± SD)

| | Day 20 | Day 35 |
|---|---|---|
| WT control | 7.25 ± 0.92 | 6.89 ± 0.75 |
| CIDP + vehicle | 3.86 ± 0.50 | 4.09 ± 0.47 |
| CIDP + AAT | 3.16 ± 0.95 | 5.71 ± 0.41 |

### Compound Muscle Action Potential

A strong and significant decrease of CMAP amplitude was observed in both CIDP groups compared to the wild type control group on Day 20.

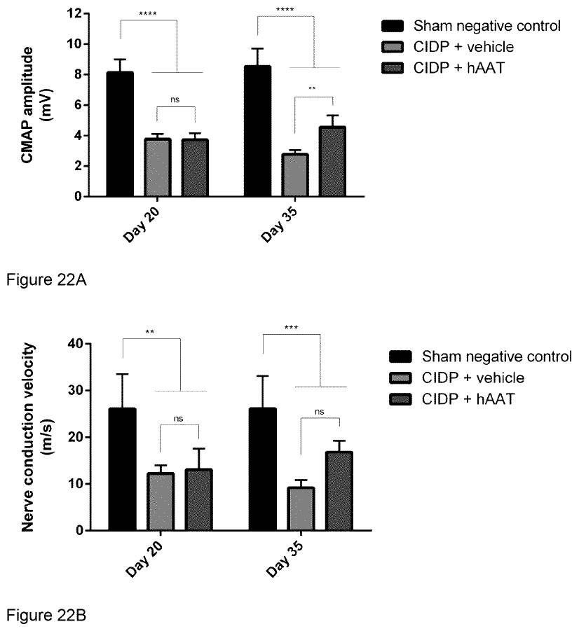

At Day 35, the CIDP+AAT treated group presented an increase of CMAP amplitude compared to the CIDP+vehicle treated group (Table 25 and FIG. 22A).

TABLE 25
Mean compound muscle action potential
Compound muscle action potential, mV (Mean ± SD)

| | Day 20 | Day 35 |
|---|---|---|
| WT control | 8.15 ± 0.85 | 8.54 ± 1.17 |
| CIDP + vehicle | 3.77 ± 0.34 | 2.78 ± 0.28 |
| CIDP + AAT | 3.73 ± 0.42 | 4.56 ± 0.76 |

### Nerve Conduction Velocity

A strong decrease ofNCV was observed in both CIDP groups compared to the wild type control group at Day 20. The CIDP+AAT treated group presented an increase of the NCV compared to the vehicle treated group (Table 26 and FIG. 22B).

TABLE 26
Mean nerve conduction velocity
Nerve conduction velocity, m/s (Mean ± SD)

| | Day 20 | Day 35 |
|---|---|---|
| WT control | 26.11 ± 7.45 | 26.16 ± 6.99 |
| CIDP + vehicle | 12.24 ± 1.70 | 9.20 ± 1.66 |
| CIDP + AAT | 13.10 ± 4.49 | 16.85 ± 2.43 |

Neuromuscular impairment and decrease of nerve conduction amplitude and velocity were observed in the preclinical CIDP mouse model treated with vehicle, compared to the wild type control group, 20 days after the start of disease induction.

An increase of rotarod latency, grip strength, paw withdrawal threshold and nerve conduction velocity and amplitude was observed in AAT treated CIDP mice compared to the vehicle treated group.

These data suggest that AAT presents a trend to a positive effect on CIDP when administrated twice a day at 50 mg/kg by subcutaneous route in CIDP mice.

### Raw Data

TABLE 27 A
Rotarod latency

Baseline (Day 20)_Rotarod latency (seconds)

| | | | | |
|---|---|---|---|---|
| Animal | set 1 | set 2 | set 3 | Mean/animal |

Sham control group

| | | | | |
|---|---|---|---|---|
| 1.1 | 102.55 | 68.92 | 96.99 | 89.48 |
| 1.2 | 67.95 | 90.98 | 71.03 | 76.65 |
| 1.3 | 82.89 | 99.21 | 76.55 | 86.22 |
| 1.4 | 88.28 | 68.13 | 102.28 | 86.23 |
| | | | MEAN | 84.65 |
| | | | SD | 5.55 |

CIDP + vehicle

| | | | | |
|---|---|---|---|---|
| 2.1 | 36.9 | 21.6 | 20.6 | 26.34 |
| 2.2 | 28.7 | 20.9 | 40.7 | 30.12 |
| 2.3 | 25.6 | 14.3 | 26.6 | 22.18 |
| 2.4 | 26.4 | 15.8 | 12.4 | 18.20 |
| | | | MEAN | 24.21 |
| | | | SD | 5.16 |

CIDP + hAAT

| | | | | |
|---|---|---|---|---|
| 3.1 | 19.1 | 17.7 | 16.2 | 17.68 |
| 3.2 | 28.0 | 26.7 | 32.9 | 29.19 |
| 3.3 | 28.7 | 33.0 | 33.8 | 31.82 |
| 3.4 | 24.1 | 24.0 | 27.3 | 25.13 |
| | | | MEAN | 25.96 |
| | | | SD | 6.16 |

TABLE 27 B
Rotarod latency

Day 35_Rotarod latency (seconds)

| | | | | |
|---|---|---|---|---|
| Animal | set 1 | set 2 | set 3 | Mean/animal |

Sham control group

| | | | | |
|---|---|---|---|---|
| 1.1 | 90.87 | 53.01 | 71.22 | 71.70 |
| 1.2 | 71.77 | 72.47 | 95.36 | 79.87 |
| 1.3 | 109.55 | 69.15 | 85.85 | 88.18 |
| 1.4 | 98.93 | 74.19 | 85.68 | 86.27 |
| | | | MEAN | 81.50 |
| | | | SD | 7.44 |

CIDP + vehicle

| | | | | |
|---|---|---|---|---|
| 2.1 | 27.4 | 21.5 | 33.1 | 27.30 |
| 2.2 | 27.9 | 34.4 | 18.1 | 26.78 |
| 2.3 | 27.5 | 12.1 | 16.3 | 18.63 |
| 2.4 | 7.3 | 42.3 | 34.4 | 27.97 |
| | | | MEAN | 25.17 |
| | | | SD | 4.39 |

CIDP + hAAT

| | | | | |
|---|---|---|---|---|
| 3.1 | 32.02 | 36.34 | 48.35 | 38.90 |
| 3.2 | 44.79 | 31.48 | 32.56 | 36.28 |
| 3.3 | 52.44 | 34.61 | 42.63 | 43.23 |
| 3.4 | 37.74 | 19.21 | 7.29 | 21.41 |
| | | | MEAN | 34.96 |
| | | | SD | 9.47 |

TABLE 28 A
Grip strength

Baseline (Day 20)_Grip strength (Newton)

| | | | | |
|---|---|---|---|---|
| Animal | set 1 | set 2 | set 3 | Mean/animal |

Sham control group

| | | | | |
|---|---|---|---|---|
| 1.1 | 5.59 | 8.13 | 5.90 | 6.54 |
| 1.2 | 3.89 | 3.90 | 5.21 | 4.33 |
| 1.3 | 6.30 | 6.11 | 6.51 | 6.31 |
| 1.4 | 5.17 | 6.88 | 5.66 | 5.90 |
| | | | MEAN | 5.77 |
| | | | SD | 0.99 |

CIDP + vehicle

| | | | | |
|---|---|---|---|---|
| 2.1 | 3.29 | 2.17 | 3.31 | 2.93 |
| 2.2 | 3.02 | 3.92 | 2.31 | 3.08 |
| 2.3 | 3.10 | 1.83 | 2.01 | 2.31 |
| 2.4 | 1.54 | 4.44 | 3.65 | 3.21 |
| | | | MEAN | 2.88 |
| | | | SD | 0.40 |

CIDP + hAAT

| | | | | |
|---|---|---|---|---|
| 3.1 | 2.92 | 2.43 | 2.04 | 2.46 |
| 3.2 | 3.06 | 2.09 | 4.42 | 3.19 |
| 3.3 | 2.89 | 2.45 | 3.14 | 2.83 |
| 3.4 | 3.50 | 2.62 | 3.13 | 3.08 |
| | | | MEAN | 2.89 |
| | | | SD | 0.32 |

TABLE 28 B
Grip strength

Day 35_Grip strength (Newton)

| | | | | |
|---|---|---|---|---|
| Animal | set 1 | set 2 | set 3 | Mean/animal |

Sham control group

| | | | | |
|---|---|---|---|---|
| 1.1 | 5.57 | 6.28 | 4.19 | 5.34 |
| 1.2 | 5.84 | 5.67 | 7.70 | 6.40 |
| 1.3 | 6.46 | 5.98 | 5.37 | 5.94 |
| 1.4 | 4.64 | 6.00 | 6.86 | 5.84 |
| | | | MEAN | 5.88 |
| | | | SD | 0.43 |

CIDP + vehicle

| | | | | |
|---|---|---|---|---|
| 2.1 | 1.76 | 3.16 | 3.63 | 2.85 |
| 2.2 | 3.24 | 1.67 | 2.60 | 2.50 |
| 2.3 | 3.47 | 2.36 | 1.99 | 2.61 |
| 2.4 | 2.58 | 2.51 | 3.72 | 2.94 |
| | | | MEAN | 2.72 |
| | | | SD | 0.20 |

CIDP + hAAT

| | | | | |
|---|---|---|---|---|
| 3.1 | 4.92 | 3.53 | 2.58 | 3.68 |
| 3.2 | 4.55 | 2.00 | 4.52 | 3.69 |
| 3.3 | 5.15 | 3.04 | 3.53 | 3.91 |
| 3.4 | 3.47 | 2.61 | 3.23 | 3.10 |
| | | | MEAN | 3.59 |
| | | | SD | 0.35 |

TABLE 29 A
Von Frey test
Baseline (Day 20)_von Frey test (Paw withdrawal threshold in grams)

| | | | | |
|---|---|---|---|---|
| Animal | set 1 | set 2 | set 3 | Mean/animal |

Sham control group

| | | | | |
|---|---|---|---|---|
| 1.1 | 6.6 | 9.3 | 8.0 | 7.97 |
| 1.2 | 7.3 | 8.3 | 6.8 | 7.47 |
| 1.3 | 6.1 | 4.0 | 7.6 | 5.90 |
| 1.4 | 7.0 | 8.6 | 7.4 | 7.66 |
| | | | MEAN | 7.25 |
| | | | SD | 0.92 |

CIDP + vehicle

| | | | | |
|---|---|---|---|---|
| 2.1 | 3.0 | 3.9 | 4.3 | 3.74 |
| 2.2 | 2.9 | 3.3 | 3.6 | 3.28 |
| 2.3 | 3.0 | 4.3 | 4.4 | 3.92 |
| 2.4 | 3.7 | 2.8 | 6.9 | 4.49 |
| | | | MEAN | 3.86 |
| | | | SD | 0.50 |

CIDP + hAAT

| | | | | |
|---|---|---|---|---|
| 3.1 | 4.6 | 2.4 | 6.0 | 4.30 |
| 3.2 | 2.1 | 1.8 | 2.4 | 2.10 |
| 3.3 | 3.3 | 2.1 | 2.8 | 2.74 |
| 3.4 | 4.5 | 2.2 | 3.8 | 3.48 |
| | | | MEAN | 3.16 |
| | | | SD | 0.95 |

TABLE 29 B
Von Frey test
Day 35_von Frey test (Paw withdrawal threshold in grams)

Sham control group

| Animal | set 1 | set 2 | set 3 | Mean/animal |
|---|---|---|---|---|
| 1.1 | 7.0 | 9.5 | 4.5 | 7.02 |
| 1.2 | 6.1 | 6.3 | 6.6 | 6.34 |
| 1.3 | 5.3 | 8.0 | 6.8 | 6.70 |
| 1.4 | 6.0 | 9.2 | 8.2 | 7.83 |
| | | | MEAN | 6.97 |
| | | | SD | 0.63 |

CIDP + vehicle

| Animal | set 1 | set 2 | set 3 | Mean/animal |
|---|---|---|---|---|
| 2.1 | 4.4 | 4.2 | 2.9 | 3.86 |
| 2.2 | 4.9 | 2.9 | 3.8 | 3.84 |
| 2.3 | 2.8 | 3.9 | 4.9 | 3.88 |
| 2.4 | 4.6 | 3.4 | 2.3 | 3.46 |
| | | | MEAN | 3.76 |
| | | | SD | 0.20 |

CIDP + hAAT

| Animal | set 1 | set 2 | set 3 | Mean/animal |
|---|---|---|---|---|
| 3.1 | 5.3 | 5.1 | 6.4 | 5.60 |
| 3.2 | 5.9 | 3.7 | 6.1 | 5.24 |
| 3.3 | 7.7 | 5.3 | 5.7 | 6.21 |
| 3.4 | 5.1 | 7.0 | 6.3 | 6.14 |
| | | | MEAN | 5.80 |
| | | | SD | 0.46 |

TABLE 30
Electrophysiology

| Amplitude (mV) | | T1 distal (ms) | | T2 proximal (ms) | | Conduction velocity (m/s) | |
|---|---|---|---|---|---|---|---|
| Day 20 | Day 35 | Day 20 | Day 35 | Day 20 | Day 35 | Day 20 | Day 35 |

Sham control group

| | | | | | | | | |
|---|---|---|---|---|---|---|---|---|
| Mouse 1, 1 | 7.256 | 7.815 | 3.298 | 7.273 | 3.713 | 7.964 | 36.155 | 21.697 |
| Mouse 1, 2 | 8.113 | 7.759 | 4.337 | 6.809 | 5.034 | 7.549 | 21.538 | 20.261 |
| Mouse 1, 3 | 7.932 | 8.331 | 3.538 | 5.834 | 4.307 | 6.391 | 19.510 | 26.949 |
| Mouse 1, 4 | 9.292 | 10.256 | 3.254 | 6.937 | 3.805 | 7.357 | 27.223 | 35.716 |
| MEAN | 8.148 | 8.540 | | | | | 26.106 | 26.156 |
| SD | 0.847 | 1.172 | | | | | 7.452 | 6.992 |
| SEM | 0.268 | 0.371 | | | | | 2.357 | 2.211 |

CIDP + vehicle

| | | | | | | | | |
|---|---|---|---|---|---|---|---|---|
| Mouse 2, 1 | 3.862 | 3.050 | 2.489 | 5.848 | 3.976 | 7.946 | 10.086 | 7.149 |
| Mouse 2, 2 | 3.941 | 2.482 | 3.332 | 2.859 | 4.592 | 4.197 | 11.898 | 11.211 |
| Mouse 2, 3 | 3.267 | 2.604 | 1.869 | 2.222 | 2.932 | 3.863 | 14.113 | 9.142 |
| Mouse 2, 4 | 4.024 | 2.978 | 3.733 | 2.462 | 4.899 | 4.074 | 12.866 | 9.308 |
| MEAN | 3.773 | 2.778 | | | | | 12.241 | 9.203 |
| SD | 0.344 | 0.278 | | | | | 1.699 | 1.660 |
| SEM | 0.109 | 0.088 | | | | | 0.537 | 0.525 |

CIDP + hAAT

| | | | | | | | | |
|---|---|---|---|---|---|---|---|---|
| Mouse 3, 1 | 3.734 | 3.490 | 2.673 | 4.089 | 3.605 | 5.026 | 16.096 | 16.007 |
| Mouse 3, 2 | 3.562 | 5.146 | 3.177 | 3.902 | 4.533 | 4.833 | 11.057 | 16.112 |
| Mouse 3, 3 | 3.318 | 5.068 | 2.864 | 4.177 | 4.793 | 5.185 | 7.775 | 14.881 |
| Mouse 3, 4 | 4.308 | 4.527 | 3.114 | 3.970 | 3.973 | 4.705 | 17.463 | 20.402 |
| MEAN | 3.731 | 4.558 | | | | | 13.098 | 16.851 |
| SD | 0.421 | 0.763 | | | | | 4.492 | 2.432 |
| SEM | 0.133 | 0.241 | | | | | 1.421 | 0.769 |

TABLE 31A
Bodyweight

| | days | | | | | | | | | | |
|---|---|---|---|---|---|---|---|---|---|---|---|
| Animal number | 1 | 5 | 9 | 13 | 17 | 20 | 23 | 26 | 29 | 32 | 35 |

Sham negative control

| | | | | | | | | | | | |
|---|---|---|---|---|---|---|---|---|---|---|---|
| 1.1 | 19.40 | 19.91 | 19.98 | 20.27 | 20.66 | 20.86 | 21.14 | 21.24 | 21.97 | 22.27 | 22.96 |
| 1.2 | 21.26 | 21.36 | 21.90 | 22.36 | 23.26 | 23.46 | 23.61 | 23.78 | 24.34 | 25.16 | 25.79 |
| 1.3 | 20.69 | 20.88 | 21.08 | 21.50 | 21.97 | 22.21 | 22.70 | 23.28 | 23.85 | 24.10 | 25.02 |
| 1.4 | 19.48 | 19.98 | 20.48 | 20.49 | 20.93 | 21.10 | 21.46 | 22.09 | 23.04 | 23.98 | 24.35 |

CIDP + vehicle

| | | | | | | | | | | | |
|---|---|---|---|---|---|---|---|---|---|---|---|
| 2.1 | 19.73 | 20.52 | 21.02 | 21.32 | 21.41 | 21.42 | 21.56 | 21.69 | 21.70 | 21.86 | 21.97 |
| 2.2 | 20.77 | 20.81 | 21.64 | 21.87 | 21.94 | 21.97 | 22.04 | 22.21 | 22.39 | 22.43 | 22.58 |
| 2.3 | 20.28 | 21.05 | 21.77 | 21.28 | 22.31 | 22.47 | 22.66 | 22.83 | 22.83 | 22.89 | 22.97 |
| 2.4 | 19.45 | 19.74 | 19.80 | 20.06 | 20.62 | 20.77 | 20.85 | 20.96 | 21.14 | 21.15 | 21.19 |

CIDP + hAAT

| | | | | | | | | | | | |
|---|---|---|---|---|---|---|---|---|---|---|---|
| 3.1 | 20.74 | 20.92 | 21.26 | 21.76 | 21.92 | 22.06 | 22.21 | 22.31 | 22.49 | 22.68 | 22.79 |
| 3.2 | 20.81 | 21.07 | 21.26 | 21.37 | 21.54 | 21.58 | 21.71 | 21.78 | 21.87 | 22.00 | 22.14 |
| 3.3 | 19.50 | 20.12 | 20.54 | 20.24 | 21.35 | 21.41 | 21.43 | 21.47 | 21.53 | 21.64 | 21.65 |
| 3.4 | 20.65 | 20.71 | 21.00 | 21.18 | 21.90 | 22.04 | 22.13 | 22.20 | 22.30 | 22.45 | 22.62 |

### Histology

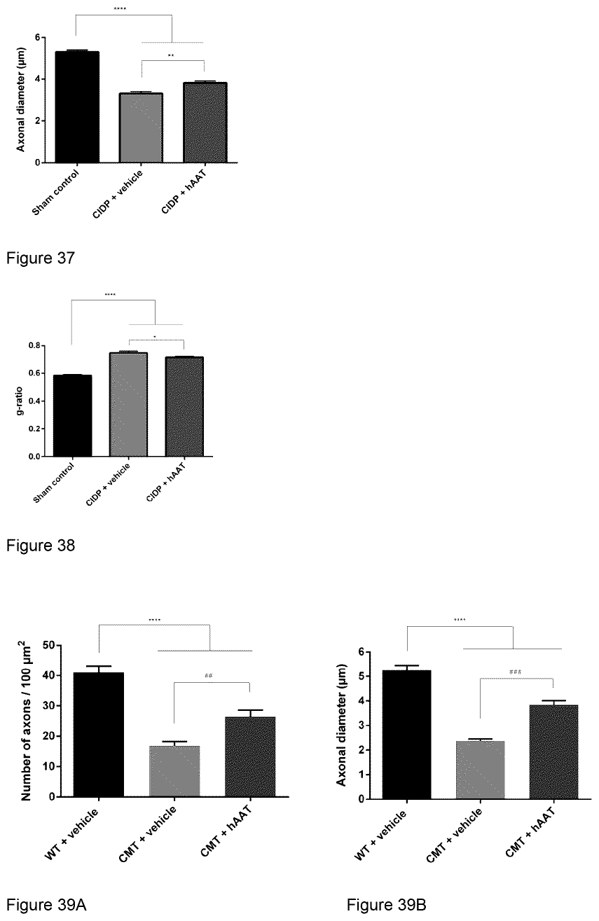

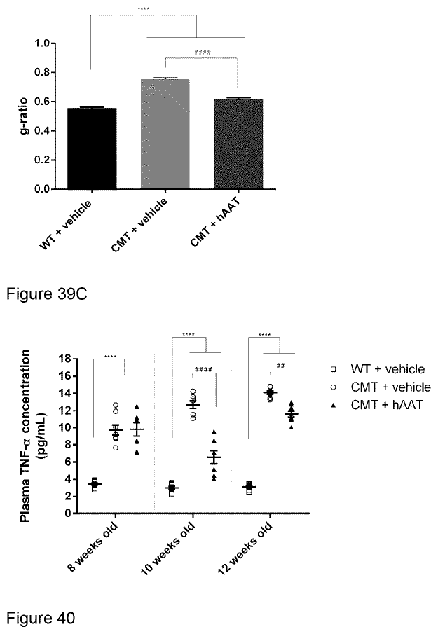

A significant decrease of the total number of axons and the axonal diameter and significant increase of the g-ratio was observed in the CIDP+vehicle group compared to the sham control group at day 35 (Table 31B and FIG. 37 to FIG. 39C). Slight but non-significant increase of the total number of axons per surface was observed in the CIDP+AAT treated group compared to the vehicle treated group. A significant increase of the axonal diameter and decrease of the g-ratio was also observed in the CIDP+AAT treated group compared to the CIDP+vehicle treated group at day 35.

TABLE 31B
Sciatic nerve histology
Sciatic nerve histology (Mean ± SEM)

| | Axons/100 μm² | Axonal diameter (μm) | g-ratio |
|---|---|---|---|
| WT control | 51.40 ± 3.90 | 5.30 ± 0.08 | 0.585 ± 0.060 |
| CIDP + vehicle | 19.20 ± 1.00 | 3.31 ± 0.10 | 0.749 ± 0.012 |
| CIDP + AAT | 26.00 ± 2.10 | 3.82 ± 0.09 | 0.715 ± 0.008 |

### Example 18: Plasma NfL Quantification

Similar plasma NfL concentrations were observed between the three groups at the baseline (day 1, before model induction).

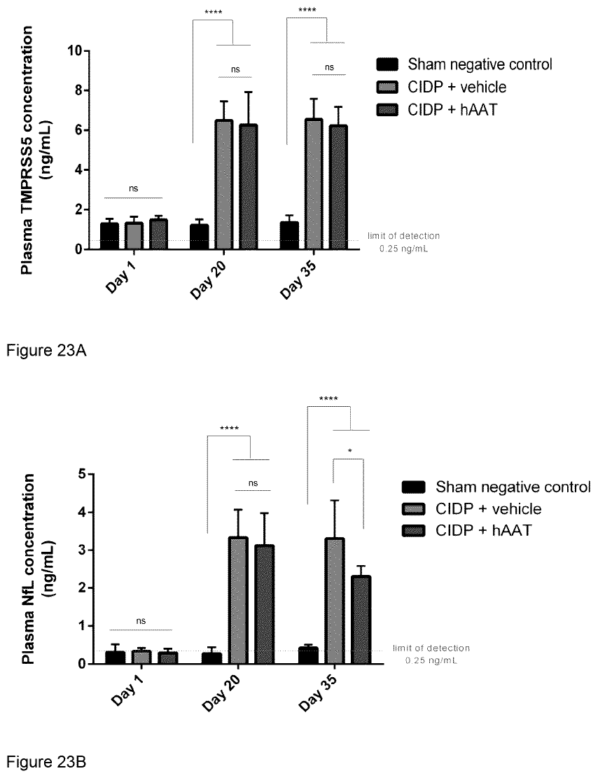

A significant increase of plasma NfL concentration was observed in the CIDP+vehicle and CIDP+AAT groups compared to the sham control group before compound treatment at day 20 (Table 32, Table 33 and FIG. 23B).

Importantly, significant decrease of plasma NfL concentration was observed in the CIDP+AAT treated group compared to the CIDP+vehicle treated group at day 35 (Table 32, Table 33 and FIG. 23B) confirming the positive efficacy of the compound on the peripheral axonopathy induced by this inflammatory neuropathy at these experimental conditions.

### Example 19: Plasma TNFα Quantification

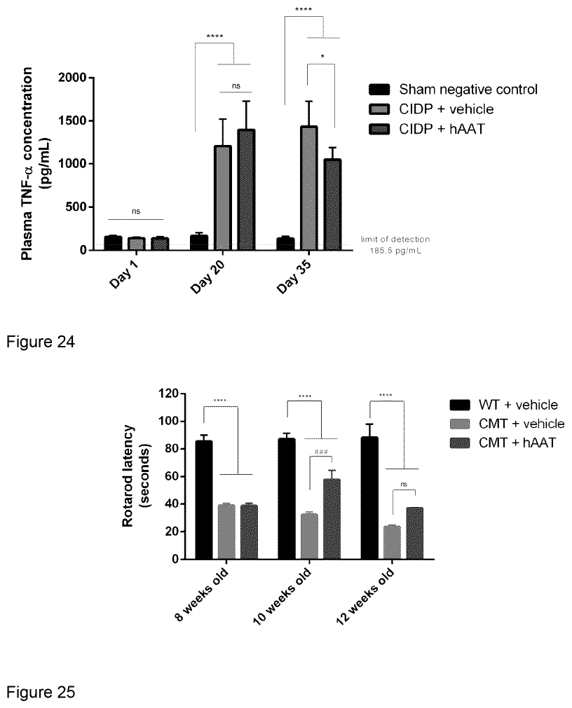

Similar plasma TNFα concentration was observed between the three groups at the baseline (day 1, before model induction). A significant increase of plasma TNFα concentration was observed in the CIDP+vehicle and CIDP+AAT groups compared to the sham control group before compound treatment at day 20 (Table 34, Table 35 and FIG. 24).

Importantly, significant decrease of plasma TNFα concentration was observed in the CIDP+AAT treated group compared to the CIDP+vehicle treated group at day 35 (Table 34, Table 35 and FIG. 24) confirming the positive efficacy of the compound on the cytokines activation induced by the autoimmune chronic inflammatory demyelinating polyneuropathy at these experimental conditions.

In conclusion neuromuscular impairment and decrease of nerve conduction amplitude and velocity were observed in the preclinical CIDP mouse model treated with vehicle, compared to the wild-type control group, 20 days after the start of disease induction.

An increase of rotarod latency, grip strength and nerve conduction velocity were observed in CIDP+AAT treated group compared to the CIDP+vehicle treated group. No statistically significant differences were observed in the plasma TMPRSS5 concentration of CIDP+AAT treated group compared to the vehicle group. However, significant increase of compound muscle action potential amplitude, decrease of neuropathic pain and decrease of plasma TNFα and NfL concentration was observed in CIDP+AAT treated group compared to the CIDP+vehicle treated group at day 35. Finally, the histopathology analysis of sciatic nerve showed a significant increase of the axonal diameter and myelin sheath diameter (decrease of g-ratio) in the CIDP+AAT compared the vehicle treated group confirming the positive efficacy of the compound from a histological point of view.

Taken together these data suggest that AAT presents positive efficacy on the autoimmune chronic inflammatory demyelinating polyneuropathy targeting peripheral nervous system, increasing neuromuscular and electrophysiological performances and decreasing neuropathic pain and plasma neuropathy biomarkers when administrated twice a day at 50 mg/kg by subcutaneous route in a preclinical CIDP mouse model.

TABLE 32 A

| | Day 1 NfL concentration (ng/mL) | | | | | Day 20 NfL concentration (ng/mL) | | | | | Day 35 NfL concentration (ng/mL) | | | |
|---|---|---|---|---|---|---|---|---|---|---|---|---|---|---|
| | Sham control group | | | | | Sham control group | | | | | Sham control group | | | |
| | | | Mean | | | | | Mean | | | | | Mean | |
| | | | abs. | NfL | | | | abs. | NfL | | | | abs. | NfL |
| Animal | | | dilution | conc | Animal | | | dilution | conc | Animal | | | dilution | conc |
| number | Abs 1 | Abs 2 | corrected | (ng/mL) | number | Abs 1 | Abs 2 | corrected | (ng/mL) | number | Abs 1 | Abs 2 | corrected | (ng/mL) |
| 1.1 | 0.097 | 0.098 | 0.097 | 0.534 | 1.1 | 0.0490 | 0.0492 | 0.0491 | 0.2695 | 1.1 | 0.0989 | 0.0995 | 0.0992 | 0.5442 |
| 1.2 | 0.075 | 0.075 | 0.075 | 0.410 | 1.2 | 0.0821 | 0.0827 | 0.0824 | 0.4520 | 1.2 | 0.0694 | 0.0697 | 0.0695 | 0.3814 |
| 1.3 | 0.044 | 0.045 | 0.045 | 0.244 | 1.3 | 0.0075 | 0.0077 | 0.0076 | 0.0417 | 1.3 | 0.0626 | 0.0630 | 0.0628 | 0.3446 |
| 1.4 | 0.011 | 0.011 | 0.011 | 0.060 | 1.4 | 0.0561 | 0.0568 | 0.0565 | 0.3097 | 1.4 | 0.0777 | 0.0784 | 0.0780 | 0.4281 |
| | | | MEAN | 0.31 | | | | MEAN | 0.27 | | | | MEAN | 0.42 |
| | | | SD | 0.21 | | | | SD | 0.17 | | | | SD | 0.09 |

| | CIDP + vehicle | | | | | CIDP + vehicle | | | | | CIDP + vehicle | | | |
|---|---|---|---|---|---|---|---|---|---|---|---|---|---|---|
| | | | Mean | | | | | Mean | | | | | Mean | |
| | | | abs. | NfL | | | | abs. | NfL | | | | abs. | NfL |
| Animal | | | dilution | conc | Animal | | | dilution | conc | Animal | | | dilution | conc |
| number | Abs 1 | Abs 2 | corrected | (ng/mL) | number | Abs 1 | Abs 2 | corrected | (ng/mL) | number | Abs 1 | Abs 2 | corrected | (ng/mL) |
| 2.1 | 0.0466 | 0.0457 | 0.0462 | 0.2533 | 2.1 | 0.4814 | 0.4847 | 0.4831 | 2.6499 | 2.1 | 0.4774 | 0.4687 | 0.4730 | 2.5947 |
| 2.2 | 0.0664 | 0.0664 | 0.0664 | 0.3641 | 2.2 | 0.5704 | 0.5701 | 0.5703 | 3.1282 | 2.2 | 0.8700 | 0.8780 | 0.8740 | 4.7942 |
| 2.3 | 0.0559 | 0.0553 | 0.0556 | 0.3049 | 2.3 | 0.7971 | 0.8034 | 0.8003 | 4.3898 | 2.3 | 0.5035 | 0.5082 | 0.5059 | 2.7749 |
| 2.4 | 0.0813 | 0.0804 | 0.0809 | 0.4437 | 2.4 | 0.5717 | 0.5805 | 0.5761 | 3.1601 | 2.4 | 0.5544 | 0.5616 | 0.5580 | 3.0609 |
| | | | MEAN | 0.34 | | | | MEAN | 3.33 | | | | MEAN | 3.31 |
| | | | SD | 0.08 | | | | SD | 0.74 | | | | SD | 1.01 |
| | CIDP + hAAT | | | | | CIDP + hAAT | | | | | CIDP + hAAT | | | |
| | | | Mean | | | | | Mean | | | | | Mean | |
| | | | abs. | NfL | | | | abs. | NfL | | | | abs. | NfL |
| Animal | | | dilution | conc | Animal | | | dilution | conc | Animal | | | dilution | conc |
| number | Abs 1 | Abs 2 | corrected | (ng/mL) | number | Abs 1 | Abs 2 | corrected | (ng/mL) | number | Abs 1 | Abs 2 | corrected | (ng/mL) |
| 3.1 | 0.0662 | 0.0670 | 0.0666 | 0.3654 | 3.1 | 0.6843 | 0.6865 | 0.6854 | 3.7596 | 3.1 | 0.4437 | 0.4476 | 0.4457 | 2.4447 |
| 3.2 | 0.0521 | 0.0523 | 0.0522 | 0.2865 | 3.2 | 0.5055 | 0.5028 | 0.5041 | 2.7654 | 3.2 | 0.3484 | 0.3449 | 0.3467 | 1.9017 |
| 3.3 | 0.0692 | 0.0692 | 0.0692 | 0.3798 | 3.3 | 0.7083 | 0.7080 | 0.7082 | 3.8846 | 3.3 | 0.4268 | 0.4299 | 0.4284 | 2.3497 |
| 3.4 | 0.0253 | 0.0262 | 0.0257 | 0.1412 | 3.4 | 0.3781 | 0.3814 | 0.3797 | 2.0831 | 3.4 | 0.4520 | 0.4615 | 0.4568 | 2.5056 |
| | | | MEAN | 0.29 | | | | MEAN | 3.12 | | | | MEAN | 2.30 |
| | | | SD | 0.11 | | | | SD | 0.86 | | | | SD | 0.27 |

TABLE 32B
Plasma NfL concentration, ng/mL (Mean ± SD)

| | Day 1 | Day 20 | Day 35 |
|---|---|---|---|
| WT control | 0.31 ± 0.21 | 0.27 ± 0.17 | 0.42 ± 0.09 |
| CIDP + vehicle | 0.34 ± 0.08 | 3.33 ± 0.74 | 3.31 ± 1.01 |
| CIDP + AAT | 0.29 ± 0.11 | 3.12 ± 0.86 | 2.30 ± 0.27 |

TABLE 33
Two-way ANOVA

| Source of | % of total | | P value | | |
|---|---|---|---|---|---|
| Variation | variation | P value | summary | Significant? | |
| Interaction | 18.94 | <0.0001 | **** | Yes | |
| Row Factor | 35.61 | <0.0001 | **** | Yes | |
| Column Factor | 35.48 | <0.0001 | **** | Yes | |
| ANOVA table | SS | DF | MS | F (DFn. DFd) | P value |
| Interaction | 14.11 | 4 | 3 529 | F (4.27) = 12.82 | P < 0.0001 |
| Row Factor | 26.53 | 2 | 13.27 | F (2.27) = 48.21 | P < 0.0001 |
| Column Factor | 26.44 | 2 | 13.22 | F (2.27) = 48.05 | P < 0.0001 |
| Residual | 7 429 | 27 | 0.2752 | | |

Bonferroni's multiple comparisons test

| Groups | Mean Diff. | 95% CI of diff. | Significant? | Summary |
|---|---|---|---|---|
| Day 1 | | | | |
| Sham negative control vs. CIDP + vehicle | −0.0295 | −0.9763 to 0.9173 | No | ns |
| Sham negative control vs. CIDP + hAAT | 0.01878 | −0.9280 to 0.9655 | No | ns |
| CIDP + vehicle vs. CIDP + hAAT | 0.04828 | −0.8985 to 0.9950 | No | ns |
| Day 20 | | | | |
| Sham negative control vs. CIDP + vehicle | −3064 | −4.011 to −2.117 | Yes | **** |
| Sham negative control vs. CIDP + hAAT | −2855 | −3.802 to −1.908 | Yes | **** |
| CIDP + vehicle vs. CIDP + hAAT | 0.2088 | −0.7379 to 1.156 | No | ns |
| Day 35 | | | | |
| Sham negative control vs. CIDP + vehicle | −2882 | −3.828 to −1.935 | Yes | **** |
| Sham negative control vs. CIDP + hAAT | −1876 | −2.823 to −0.9291 | Yes | **** |
| CIDP + vehicle vs. CIDP + hAAT | 1006 | 0.05900 to 1.953 | Yes | * |

TABLE 34 A

| | Day 1 TNF-α | | | | | Day 20 TNF-α | | | | | Day 35 TNF-α | | | |
|---|---|---|---|---|---|---|---|---|---|---|---|---|---|---|
| | concentration (pg/mL) | | | | | concentration (pg/mL) | | | | | concentration (pg/mL) | | | |
| | Sham control group | | | | | Sham control group | | | | | Sham control group | | | |
| | | | Mean | | | | | Mean | | | | | Mean | |
| | | | abs. | TNFα | | | | abs. | TNFα | | | | abs. | TNFα |
| Animal | | | dilution | conc | Animal | | | dilution | conc | Animal | | | dilution | conc |
| number | Abs 1 | Abs 2 | corr | (pg/mL) | number | Abs 1 | Abs 2 | corr | (pg/mL) | number | Abs 1 | Abs 2 | corr | (pg/mL) |
| 1.1 | 0.0496 | 0.0497 | 0.0496 | 165.4920 | 1.1 | 0.0516 | 0.0517 | 0.0516 | 172.0910 | 1.1 | 0.0462 | 0.0465 | 0.0464 | 154.5570 |
| 1.2 | 0.0518 | 0.0513 | 0.0516 | 171.8472 | 1.2 | 0.0638 | 0.0641 | 0.0640 | 213.2430 | 1.2 | 0.0379 | 0.0380 | 0.0380 | 126.5980 |
| 1.3 | 0.0422 | 0.0423 | 0.0422 | 140.7485 | 1.3 | 0.0495 | 0.0485 | 0.0490 | 163.3830 | 1.3 | 0.0482 | 0.0486 | 0.0484 | 161.3470 |
| 1.4 | 0.0429 | 0.0430 | 0.0430 | 143.2007 | 1.4 | 0.0370 | 0.0377 | 0.0373 | 124.4260 | 1.4 | 0.0315 | 0.0314 | 0.0315 | 104.8520 |
| | | | MEAN | 155.32 | | | | MEAN | 168.29 | | | | MEAN | 136.84 |
| | | | SD | 15.66 | | | | SD | 36.44 | | | | SD | 26.09 |

| | CIDP + vehicle | | | | | CIDP + vehicle | | | | | CIDP + vehicle | | | |
|---|---|---|---|---|---|---|---|---|---|---|---|---|---|---|
| | | | Mean | | | | | Mean | | | | | Mean | |
| | | | abs. | TNFα | | | | abs. | TNFα | | | | abs. | TNFα |
| Animal | | | dilution | conc | Animal | | | dilution | conc | Animal | | | dilution | conc |
| number | Abs 1 | Abs 2 | corr | (pg/mL) | number | Abs 1 | Abs 2 | corr | (pg/mL) | number | Abs 1 | Abs 2 | corr | (pg/mL) |
| 2.1 | 0.0411 | 0.0412 | 0.0411 | 137.137 | 2.1 | 0.3610 | 0.3619 | 0.3615 | 1204.851 | 2.1 | 0.4738 | 0.4792 | 0.4765 | 1588.406 |
| 2.2 | 0.0461 | 0.0462 | 0.0461 | 153.786 | 2.2 | 0.2453 | 0.2481 | 0.2467 | 822.406 | 2.2 | 0.5038 | 0.5012 | 0.5025 | 1674.969 |
| 2.3 | 0.0381 | 0.0383 | 0.0382 | 127.435 | 2.3 | 0.4717 | 0.4777 | 0.4747 | 1582.443 | 2.3 | 0.2987 | 0.3065 | 0.3026 | 1008.676 |
| 2.4 | 0.0444 | 0.0441 | 0.0442 | 147.369 | 2.4 | 0.3650 | 0.3725 | 0.3687 | 1229.160 | 2.4 | 0.4325 | 0.4408 | 0.4366 | 1455.462 |
| | | | MEAN | 141.43 | | | | MEAN | 1209.71 | | | | MEAN | 1431.88 |
| | | | SD | 11.58 | | | | SD | 310.56 | | | | SD | 296.23 |
| | CIDP + hAAT | | | | | CIDP + hAAT | | | | | CIDP + hAAT | | | |
| | | | Mean | | | | | Mean | | | | | Mean | |
| | | | abs. | TNFα | | | | abs. | TNFα | | | | abs. | TNFα |
| Animal | | | dilution | conc | Animal | | | dilution | conc | Animal | | | dilution | conc |
| number | Abs 1 | Abs 2 | corr | (pg/mL) | number | Abs 1 | Abs 2 | corr | (pg/mL) | number | Abs 1 | Abs 2 | corr | (pg/mL) |
| 3.1 | 0.0374 | 0.0378 | 0.0376 | 125.451 | 3.1 | 0.5123 | 0.5112 | 0.5118 | 1705.849 | 3.1 | 0.2735 | 0.2731 | 0.2733 | 910.997 |
| 3.2 | 0.0498 | 0.0495 | 0.0497 | 165.501 | 3.2 | 0.3059 | 0.3116 | 0.3088 | 1029.281 | 3.2 | 0.3402 | 0.3566 | 0.3484 | 1161.216 |
| 3.3 | 0.0407 | 0.0408 | 0.0407 | 135.780 | 3.3 | 0.3606 | 0.3645 | 0.3625 | 1208.445 | 3.3 | 0.2785 | 0.2814 | 0.2799 | 933.130 |
| 3.4 | 0.0377 | 0.0386 | 0.0381 | 127.127 | 3.4 | 0.4897 | 0.4985 | 0.4941 | 1646.907 | 3.4 | 0.3569 | 0.3520 | 0.3545 | 1181.571 |
| | | | MEAN | 138.46 | | | | MEAN | 1397.62 | | | | MEAN | 1046.73 |
| | | | SD | 18.58 | | | | SD | 330.96 | | | | SD | 144.47 |

TABLE 34B
Plasma TNFα concentration, pg/mL (Mean ± SD)

| | Day 1 | Day 20 | Day 35 |
|---|---|---|---|
| WT control | 155.32 ± 15.66 | 168.29 ± 36.44 | 136.84 ± 26.09 |
| CIDP + vehicle | 141.43 ± 11.58 | 1209.71 ± 310.56 | 1431.88 ± 296.23 |
| CIDP + AAT | 138.46 ± 18.58 | 1397.62 ± 330.96 | 1046.73 ± 144.47 |

TABLE 35
Two-way ANOVA

| Source of | % of total | | P value | | |
|---|---|---|---|---|---|
| Variation | variation | P value | summary | Significant? | |
| Interaction | 20.94 | <0.0001 | **** | Yes | |
| Row Factor | 36.3 | <0.0001 | **** | Yes | |
| Column Factor | 35.18 | <0.0001 | **** | Yes | |
| ANOVA table | SS | DF | MS | F (DFn. DFd) | P value |
| Interaction | 2629000 | 4 | 657345 | F (4.27) = 18.64 | P < 0.0001 |
| Row Factor | 4558000 | 2 | 2279000 | F (2.27) = 64.64 | P < 0.0001 |
| Column Factor | 4417000 | 2 | 2209000 | F (2.27) = 62.64 | P < 0.0001 |
| Residual | 952014 | 27 | 35260 | | |

Bonferroni's multiple comparisons test

| Groups | Mean Diff. | 95% CI of diff. | Significant? | Summary |
|---|---|---|---|---|
| Day 1 | | | | |
| Sham negative control vs. CIDP | 13.89 | −325.0 to 352.8 | No | ns |
| Sham negative control vs. CIDP | 16.86 | −322.1 to 355.8 | No | ns |
| CIDP + vehicle vs. CIDP + hAAT | 2 967 | −335.9 to 341.9 | No | ns |
| Day 20 | | | | |
| Sham negative control vs. CIDP | −1041 | −1380 to −702.5 | Yes | **** |
| Sham negative control vs. CIDP | −1229 | −1568 to −890.4 | Yes | **** |
| CIDP + vehicle vs. CIDP + hAAT | −187.9 | −526.8 to 151.0 | No | ns |
| Day 35 | | | | |
| Sham negative control vs. CIDP | −1295 | −1634 to −956.1 | Yes | **** |
| Sham negative control vs. CIDP | −909.9 | −1249 to −571.0 | Yes | **** |
| CIDP + vehicle vs. CIDP + hAAT | 385.1 | 46.24 to 724.1 | Yes | * |

### Example 20

Compound Muscle Action Potential (CMAP) significantly increases after two weeks of AAT therapy.

CMT MOUSE MODEL: Non-humanized CMT1A mice B6.Cg-Tg(PMP22)C3Fbas/J (three copies of the PMP22 gene), three groups of 8 animals each.

DOSAGE: 180 mg/kg/day non-clinical grade AAT (Sigma)

ADMINISTRATION: Subcutaneous (SC) (bi-daily injection of 90 mg/kg)

Key disease progression parameters significantly improve after two weeks of AAT treatment (180 mg/kg/day) see FIG. 25-30.

TABLE 36
Rotarod latency

| Rotarod latency at baseline (6 weeks old) (in SECONDS) | | | | | Rotarod latency at 8 weeks old (in SECONDS) | | | | |
|---|---|---|---|---|---|---|---|---|---|
| | WT + vehicle | | | | | WT + vehicle | | | |
| Animal number | set 1 | set 2 | set 3 | Mean/animal | Animal number | set 1 | set 2 | set 3 | Mean/animal |
| 1.1 | 110.71 | 102.76 | 80.81 | 98.09 | 1.1 | 67.78 | 72.14 | 62.65 | 67.52 |
| 1.2 | 79.52 | 64.63 | 78.92 | 74.35 | 1.2 | 69.78 | 62.80 | 62.61 | 65.06 |
| 1.3 | 105.19 | 100.84 | 101.23 | 102.42 | 1.3 | 84.19 | 84.84 | 76.98 | 82.00 |
| 1.4 | 99.55 | 66.29 | 85.74 | 83.86 | 1.4 | 74.26 | 71.83 | 68.83 | 71.64 |
| 1.5 | 89.53 | 73.16 | 108.00 | 90.23 | 1.5 | 82.04 | 89.50 | 75.59 | 82.38 |
| 1.6 | 74.92 | 91.57 | 68.16 | 78.21 | 1.6 | 83.77 | 82.87 | 77.37 | 81.34 |
| 1.7 | 86.69 | 61.17 | 74.64 | 74.16 | 1.7 | 119.07 | 115.57 | 116.66 | 117.10 |
| 1.8 | 98.01 | 67.17 | 98.26 | 87.81 | 1.8 | 113.70 | 116.61 | 118.35 | 116.22 |
| | | | Mean | 86.14 | | | | Mean | 85.41 |
| | | | SD | 10.54 | | | | SD | 20.40 |

| | CMT + vehicle | | | | | CMT + vehicle | | | |
|---|---|---|---|---|---|---|---|---|---|
| Animal number | set 1 | set 2 | set 3 | Mean/animal | Animal number | set 1 | set 2 | set 3 | Mean/animal |
| 2.1 | 48.42 | 85.90 | 68.19 | 67.50 | 2.1 | 29.63 | 47.21 | 34.04 | 36.96 |
| 2.2 | 29.20 | 32.19 | 45.39 | 35.59 | 2.2 | 27.67 | 46.34 | 14.19 | 29.40 |
| 2.3 | 72.34 | 53.17 | 70.72 | 65.41 | 2.3 | 25.63 | 63.48 | 30.51 | 39.87 |
| 2.4 | 50.97 | 34.95 | 101.40 | 62.44 | 2.4 | 45.78 | 48.26 | 29.22 | 41.09 |
| 2.5 | 118.89 | 50.77 | 112.19 | 93.95 | 2.5 | 23.74 | 61.79 | 24.54 | 36.69 |
| 2.6 | 110.30 | 108.33 | 99.29 | 105.98 | 2.6 | 40.17 | 59.89 | 29.58 | 43.22 |
| 2.7 | 51.52 | 73.49 | 93.01 | 72.67 | 2.7 | 26.20 | 50.80 | 23.70 | 33.57 |
| 2.8 | 66.48 | 71.66 | 92.09 | 76.74 | 2.8 | 33.95 | 55.89 | 36.66 | 42.17 |
| | | | Mean | 72.54 | | | | Mean | 37.87 |
| | | | SD | 21.17 | | | | SD | 4.69 |

| | CMT + hAAT | | | | | CMT + hAAT | | | |
|---|---|---|---|---|---|---|---|---|---|
| Animal number | set 1 | set 2 | set 3 | Mean/animal | Animal number | set 1 | set 2 | set 3 | Mean/animal |
| 3.1 | 53.47 | 57.11 | 49.72 | 53.43 | 3.1 | 55.48 | 61.08 | 72.89 | 63.15 |
| 3.2 | 57.35 | 51.61 | 49.96 | 52.98 | 3.2 | 48.88 | 61.14 | 45.41 | 51.81 |
| 3.3 | 69.94 | 67.40 | 62.87 | 66.74 | 3.3 | 60.00 | 48.80 | 53.05 | 53.95 |
| 3.4 | 62.11 | 54.79 | 53.98 | 56.96 | 3.4 | 47.76 | 53.19 | 55.82 | 52.25 |
| 3.5 | 65.03 | 72.92 | 62.30 | 66.75 | 3.5 | 30.45 | 62.45 | 22.62 | 38.51 |
| 3.6 | 66.86 | 68.48 | 61.35 | 65.56 | 3.6 | 96.29 | 79.58 | 100.40 | 92.09 |
| 3.7 | 110.28 | 109.55 | 100.14 | 106.66 | 3.7 | 74.06 | 65.45 | 82.56 | 74.02 |
| 3.8 | 93.16 | 105.36 | 109.32 | 102.61 | 3.8 | 68.88 | 74.87 | 81.93 | 75.23 |
| | | | Mean | 71.46 | | | | Mean | 62.63 |
| | | | SD | 21.27 | | | | SD | 17.07 |

TABLE 37
Grip strength

| Grip strength at baseline (6 weeks old) (in NEWTONS) | | | | | Grip strength at 8 weeks old (in NEWTONS) | | | | |
|---|---|---|---|---|---|---|---|---|---|
| | WT + vehicle | | | | | WT + vehicle | | | |
| Animal number | set 1 | set 2 | set 3 | Mean/animal | Animal number | set 1 | set 2 | set 3 | Mean/animal |
| 1.1 | 4.75 | 6.48 | 3.56 | 4.93 | 1.1 | 5.85 | 8.59 | 6.52 | 6.99 |
| 1.2 | 4.33 | 5.13 | 5.26 | 4.91 | 1.2 | 7.13 | 5.42 | 6.25 | 6.27 |
| 1.3 | 6.14 | 7.87 | 5.03 | 6.35 | 1.3 | 8.48 | 8.29 | 7.59 | 8.12 |
| 1.4 | 6.71 | 4.20 | 5.45 | 5.45 | 1.4 | 6.62 | 7.19 | 6.44 | 6.75 |
| 1.5 | 7.71 | 7.94 | 5.48 | 7.04 | 1.5 | 7.67 | 8.69 | 6.17 | 7.51 |
| 1.6 | 7.44 | 5.69 | 5.57 | 6.24 | 1.6 | 5.47 | 4.30 | 4.17 | 4.65 |
| 1.7 | 9.76 | 9.84 | 10.84 | 10.15 | 1.7 | 7.65 | 8.82 | 7.58 | 8.01 |
| 1.8 | 9.78 | 9.79 | 10.62 | 10.07 | 1.8 | 7.78 | 8.81 | 8.00 | 8.19 |
| | | | Mean | 6.89 | | | | Mean | 7.06 |
| | | | SD | 2.11 | | | | SD | 1.20 |

| | CMT + vehicle | | | | | CMT + vehicle | | | |
|---|---|---|---|---|---|---|---|---|---|
| Animal number | set 1 | set 2 | set 3 | Mean/animal | Animal number | set 1 | set 2 | set 3 | Mean/animal |
| 2.1 | 3.48 | 8.97 | 4.77 | 5.74 | 2.1 | 2.47 | 2.38 | 2.33 | 2.39 |
| 2.2 | 2.45 | 2.14 | 2.32 | 2.30 | 2.2 | 2.65 | 2.65 | 1.04 | 2.12 |
| 2.3 | 6.77 | 4.30 | 6.43 | 5.83 | 2.3 | 0.97 | 7.40 | 2.83 | 3.73 |
| 2.4 | 3.55 | 3.48 | 8.31 | 5.12 | 2.4 | 5.29 | 3.46 | 3.36 | 4.03 |
| 2.5 | 5.16 | 4.98 | 4.49 | 4.88 | 2.5 | 0.96 | 4.18 | 3.01 | 2.72 |
| 2.6 | 8.64 | 5.85 | 7.83 | 7.44 | 2.6 | 2.76 | 4.51 | 3.10 | 3.46 |
| 2.7 | 3.48 | 6.61 | 9.39 | 6.50 | 2.7 | 1.93 | 3.44 | 1.67 | 2.35 |
| 2.8 | 4.43 | 5.46 | 8.72 | 6.20 | 2.8 | 3.34 | 5.06 | 1.20 | 3.20 |
| | | | Mean | 5.50 | | | | Mean | 3.00 |
| | | | SD | 1.52 | | | | SD | 0.71 |
| | CMT + hAAT | | | | | CMT + hAAT | | | |
| Animal number | set 1 | set 2 | set 3 | Mean/animal | Animal number | set 1 | set 2 | set 3 | Mean/animal |
| 3.1 | 6.56 | 3.79 | 4.52 | 4.96 | 3.1 | 3.97 | 5.37 | 5.14 | 4.83 |
| 3.2 | 5.62 | 4.00 | 4.69 | 4.77 | 3.2 | 2.15 | 4.34 | 1.69 | 2.73 |
| 3.3 | 7.24 | 7.19 | 5.93 | 6.79 | 3.3 | 3.47 | 2.22 | 3.21 | 2.97 |
| 3.4 | 5.90 | 8.88 | 5.37 | 6.72 | 3.4 | 4.13 | 4.58 | 3.55 | 4.09 |
| 3.5 | 4.97 | 7.60 | 6.66 | 6.41 | 3.5 | 4.85 | 3.84 | 6.47 | 5.05 |
| 3.6 | 4.35 | 6.25 | 3.63 | 4.74 | 3.6 | 8.17 | 5.69 | 8.92 | 7.59 |
| 3.7 | 6.08 | 2.61 | 4.13 | 4.27 | 3.7 | 6.20 | 4.40 | 5.99 | 5.53 |
| 3.8 | 6.51 | 3.97 | 6.57 | 5.68 | 3.8 | 4.87 | 5.39 | 6.77 | 5.68 |
| | | | Mean | 5.54 | | | | Mean | 4.81 |
| | | | SD | 0.99 | | | | SD | 1.57 |

TABLE 38
Electrophysiology

| Amplitude (mV) | | T1 distal (ms) | | T2 proximal (ms) | | Conduction velocity (m/s) | |
|---|---|---|---|---|---|---|---|
| 6 weeks old | 8 weeks old | 6 weeks old | 8 weeks old | 6 weeks old | 8 weeks old | 6 weeks old | 8 weeks old |

WT + vehicle

| | | | | | | | | |
|---|---|---|---|---|---|---|---|---|
| Mouse 1, 1 | 7.404 | 7.700 | 3.959 | 4.628 | 4.296 | 4.886 | 44.476 | 58.132 |
| Mouse 1, 2 | 9.509 | 5.513 | 5.755 | 6.457 | 6.339 | 6.964 | 25.702 | 29.554 |
| Mouse 1, 3 | 5.676 | 5.971 | 4.793 | 4.463 | 5.468 | 5.057 | 22.227 | 25.237 |
| Mouse 1, 4 | 3.969 | 7.500 | 4.595 | 4.519 | 5.056 | 5.001 | 32.545 | 31.101 |
| Mouse 1, 5 | 4.658 | 6.293 | 1.995 | 3.578 | 2.526 | 4.131 | 28.220 | 27.093 |
| Mouse 1, 6 | 10.121 | 9.150 | 3.629 | 4.959 | 4.241 | 5.639 | 24.508 | 22.070 |
| Mouse 1, 7 | 6.668 | 7.655 | 5.115 | 5.394 | 5.406 | 5.740 | 51.553 | 43.468 |
| Mouse 1, 8 | 9.740 | 8.402 | 3.940 | 4.494 | 4.650 | 5.116 | 21.125 | 24.117 |
| MEAN | 7.218 | 7.273 | | | | | 31.294 | 32.597 |
| SD | 2.386 | 1.250 | | | | | 11.072 | 12.241 |

CMT + vehicle

| | | | | | | | | |
|---|---|---|---|---|---|---|---|---|
| Mouse 2, 1 | 4.758 | 2.137 | 6.881 | 1.955 | 7.521 | 3.407 | 23.447 | 10.327 |
| Mouse 2, 2 | 6.698 | 1.927 | 3.939 | 4.220 | 4.616 | 5.557 | 22.165 | 11.224 |
| Mouse 2, 3 | 7.307 | 2.943 | 2.992 | 3.423 | 3.399 | 4.807 | 36.829 | 10.845 |
| Mouse 2, 4 | 5.180 | 3.395 | 3.879 | 3.066 | 4.645 | 4.691 | 19.581 | 9.231 |
| Mouse 2, 5 | 8.049 | 4.452 | 3.120 | 3.607 | 3.764 | 4.828 | 23.288 | 12.278 |
| Mouse 2, 6 | 7.274 | 1.850 | 3.677 | 3.999 | 4.195 | 5.367 | 28.930 | 10.965 |
| Mouse 2, 7 | 7.054 | 3.429 | 3.001 | 5.501 | 3.874 | 7.161 | 17.179 | 9.039 |
| Mouse 2, 8 | 5.498 | 1.858 | 4.094 | 2.417 | 4.567 | 3.911 | 31.671 | 10.040 |
| MEAN | 6.477 | 2.749 | | | | | 25.386 | 10.494 |
| SD | 1.182 | 0.961 | | | | | 6.575 | 1.069 |

CMT + hAAT

| | | | | | | | | |
|---|---|---|---|---|---|---|---|---|
| Mouse 3, 1 | 7.828 | 4.874 | 7.416 | 3.916 | 7.885 | 4.718 | 31.949 | 18.707 |
| Mouse 3, 2 | 3.009 | 5.376 | 5.701 | 3.259 | 6.130 | 4.170 | 34.989 | 16.462 |
| Mouse 3, 3 | 5.770 | 5.322 | 12.428 | 3.449 | 12.922 | 4.945 | 30.328 | 10.029 |
| Mouse 3, 4 | 5.919 | 9.084 | 5.913 | 4.291 | 6.694 | 5.072 | 19.190 | 19.200 |
| Mouse 3, 5 | 7.925 | 6.115 | 6.041 | 3.773 | 6.869 | 4.624 | 18.114 | 17.615 |
| Mouse 3, 6 | 8.742 | 3.753 | 3.664 | 8.247 | 4.395 | 9.802 | 20.530 | 9.648 |
| Mouse 3, 7 | 6.803 | 5.277 | 4.204 | 7.467 | 4.775 | 8.057 | 26.247 | 25.446 |
| Mouse 3, 8 | 7.028 | 4.139 | 3.385 | 11.449 | 3.906 | 12.262 | 28.783 | 18.460 |
| MEAN | 6.628 | 5.492 | | | | | 26.266 | 16.946 |
| SD | 1.778 | 1.631 | | | | | 6.332 | 5.134 |

TABLE 39
TNFα

| | Plasma TNF-α concentration at 8 weeks old (in pg/mL) | | | |
|---|---|---|---|---|
| | WT + vehicle | | | |
| Animal | | | | TNF-α |
| number | Abs 1 | Abs 2 | Mean Abs | concentration (pg/mL) |
| 1.1 | 0.54375 | 0.55496 | 0.54935 | 3.92 |
| 1.2 | 0.40014 | 0.39642 | 0.39828 | 2.73 |
| 1.3 | 0.39928 | 0.43564 | 0.41746 | 2.88 |
| 1.4 | 0.38018 | 0.39604 | 0.38811 | 2.66 |
| 1.5 | 0.40955 | 0.44471 | 0.42713 | 2.96 |
| 1.6 | 0.49326 | 0.51042 | 0.50184 | 3.54 |
| 1.7 | 0.54415 | 0.54906 | 0.54661 | 3.90 |
| 1.8 | 0.40389 | 0.41290 | 0.40840 | 2.81 |
| | | | Mean | 3.18 |
| | | | SD | 0.53 |

| | CMT + vehicle | | | |
|---|---|---|---|---|
| Animal | | | | TNF-α |
| number | Abs 1 | Abs 2 | Mean Abs | concentration (pg/mL) |
| 2.1 | 1.35282 | 1.35734 | 1.35508 | 10.22 |
| 2.2 | 1.93280 | 1.92613 | 1.92946 | 14.72 |
| 2.3 | 1.44294 | 1.48421 | 1.46357 | 11.07 |
| 2.4 | 1.57975 | 1.65685 | 1.61830 | 12.28 |
| 2.5 | 1.73361 | 1.68169 | 1.70765 | 12.98 |
| 2.6 | 1.21567 | 1.21985 | 1.21776 | 9.15 |
| 2.7 | 1.51298 | 1.51888 | 1.51593 | 11.48 |
| 2.8 | 1.80332 | 1.78964 | 1.79648 | 13.68 |
| | | | Mean | 11.95 |
| | | | SD | 1.84 |
| | CMT + hAAT | | | |
| Animal | | | | TNF-α |
| number | Abs 1 | Abs 2 | Mean Abs | concentration (pg/mL) |
| 3.1 | 0.92930 | 0.96779 | 0.94855 | 7.04 |
| 3.2 | 1.07430 | 1.04042 | 1.05736 | 7.89 |
| 3.3 | 1.07575 | 1.04322 | 1.05949 | 7.91 |
| 3.4 | 0.72589 | 0.76331 | 0.74460 | 5.44 |
| 3.5 | 1.07604 | 1.04189 | 1.05897 | 7.90 |
| 3.6 | 0.71104 | 0.75262 | 0.73183 | 5.34 |
| 3.7 | 1.09751 | 1.05099 | 1.07425 | 8.02 |
| 3.8 | 0.80920 | 0.80948 | 0.80934 | 5.95 |
| | | | Mean | 6.94 |
| | | | SD | 1.18 |

### Example 21—Efficacy of AAT in a Mouse Model of Charcot-Marie-Tooth Disease

Charcot-Marie-Tooth disease (CMT) is a hereditary motor and sensory neuropathy of the peripheral nervous system characterized by a progressive loss of muscle tissue and a dysfunction of the tactile sensation in different parts of the body. Currently incurable, this disease is the most prevalent hereditary neurological disorder and affects approximately one in 2,500 people. The most common type of CMT is CMT1A, characterized by a duplication of the pmp22 gene leading to an accumulation of the pmp22 protein in the Schwann cell and progressive demyelination. PMP22 is a tetraspan glycoprotein contained in compact myelin of the peripheral nervous system. Duplication of PMP22 has been associated with the onset of Charcot-Marie-Tooth disease type TA (CMT1A).

The C3-PMP22 transgenic mice (B6.Cg-Tg(PMP22)C3Fbas/J) express three copies of a wild-type human peripheral myelin protein 22 (PMP22) gene. These mice present an age-dependent demyelinating neuropathy characterized by predominantly distal loss of strength and sensation. C3-PMP mice show no overt clinical signs at 3 weeks and develop mild neuromuscular impairment in an age-dependent manner. They have stable, low nerve conduction velocities similar to adults with human CMT1A. Myelination is delayed in these mice, and they contain reduced numbers of myelinated axons at 3 weeks of age.

In CMT1A, the major component of the disease is a developmental abnormality of myelin formation (dysmyelination). Myelin is produced by SC in the peripheral nervous system and is crucial for proper transmission of the electric impulse in the nerves. The primary dysmyelination seen in CMT1A patients leads to an abnormal SC organization around axons and a uniformly slowed nerve conduction velocity.

It has been shown that the transmembrane protein ADAM17 (also known as TACE for Tumor necrosis factor-α-converting enzyme) blocks the myelination of axons in SC by inhibiting neuregulin 1 type III signal (NRG1-III) (La Marca et al., 2011; Pisciotta et al., 2021). AAT can be a promising treatment of CMT by the modulation of NRG1-III signaling via its pharmacological inhibition of ADAM17. The inhibition of ADAM17 by AAT promotes SC myelination in the peripheral nervous system and can therefore reduce the progression of CMT disease.

In addition to its inhibitory function on ADAM17, another mechanism of AAT in the treatment of CMT is its anti-inflammatory activity. AAT exhibits broad anti-inflammatory and immunomodulatory activity. Increasing AAT serum concentration by the administration of exogenous AAT to above-normal levels is expected to be therapeutic in CMT.

B6.Cg-Tg(PMP22)C3Fbas/J transgenic mice express three copies of a wild-type human peripheral myelin protein 22 (PMP22) gene. They present an age-dependent demyelinating neuropathy characterized by predominantly distal loss of strength and sensation. These mice show no overt clinical signs at 3 weeks and develop mild neuromuscular impairment in an age-dependent manner, which are fully established at 8 weeks of age. They have stable, low nerve conduction velocities similar to adults with human CMT1A. Myelination is delayed in these mice, and they contain reduced numbers of myelinated axons.

### Materials and Methods

Animals were received at 6 weeks of age and housed under controlled conditions for the duration of the study. The study comprised two groups of 8 transgenic mice (CMT mice) each and one group of 8 age matched wild type (WT) C57B16 mice, serving as positive control. Animals were assigned to the following subcutaneous treatments twice daily at 7:00 AM and 7:00 PM for 2 weeks, starting from 8 weeks and up to 10 weeks of age:
Wild-type (WT, no disease) mice receiving vehicle, 0.9% NaCl (positive control)
CMT1A mice receiving vehicle, 0.9% NaCl (negative control)
CMT1A mice receiving AAT at 90 mg/kg per injection (daily dose 180 mg/kg)

The efficacy parameters included:
neuromuscular performance (rotarod latency to fall and grip strength test),
sciatic nerve electrophysiology (CMAP amplitude and NCV)
cytokine (IL-6 and TNFα), as well as NfL analysis
sciatic nerve histology (number of axons and axonal diameter),

On the first day of treatment (Day 1), the neuromuscular and performance of animals was tested with a rotarod test and a grip strength test, as well as sciatic nerve electrophysiology test, which measured the compound muscle action potential (CMAP) and the nerve conduction velocity (NCV). After the last treatment on Day 15, these tests were repeated. Animals were then left untreated for 2 additional weeks to evaluate the duration of the treatment effect. Following the 2-week treatment-free period until 12 weeks of age (Day 30), the behavioural and electrophysiological tests were repeated.

As supportive information for the mode of action of AAT in CMT, plasma concentration of interleukin 6 (IL-6) and TNFα were measured at Day 1 (8 weeks), Day 15 (10 weeks) and Day 30 (12 weeks). At the same timepoints, plasma levels of the biomarker of nerve damage, NfL were determined.

### Results

At 8 weeks of age (baseline), CMT mice show evident clinical signs/symptoms of the disease compared to the normal wild-type mice. The neuromuscular impairment (decreased latency to fall) can be seen in FIG. 25 and in Table 40A

TABLE 40A
Mean rotarod latency in CMT mice treated with and without AAT
Rotarod latency, seconds (Mean ± SD)

| | 8 weeks old | 10 weeks old | 12 weeks old |
|---|---|---|---|
| WT control | 85.31 ± 12.71 | 87.13 ± 11.91 | 88.20 ± 27.50 |
| CMT1A + vehicle | 39.02 ± 4.05 | 32.31 ± 5.81 | 23.56 ± 3.52 |
| CMT1A + AAT | 38.76 ± 4.90 | 57.77 ± 19.19 | 36.92 ± 1.66 |

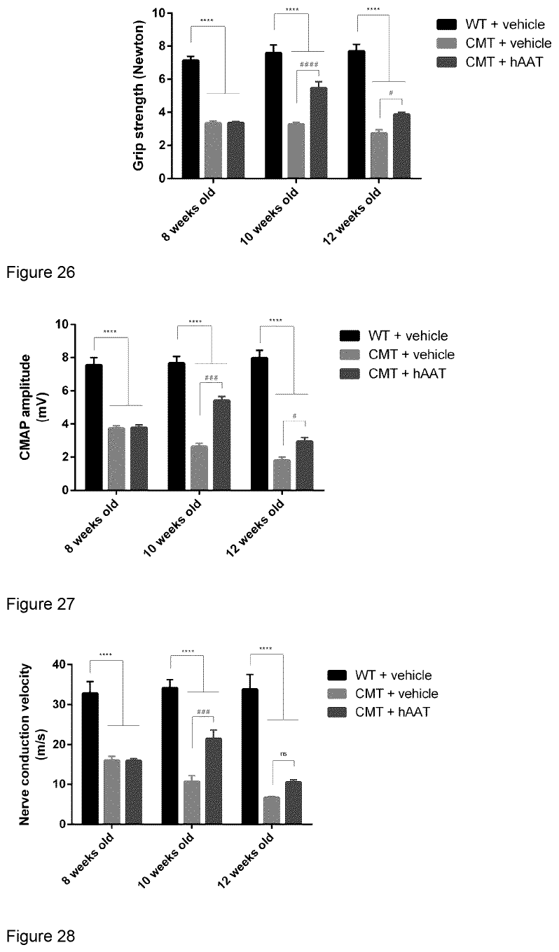

Decreased grip strength in CMT mice compared to wild-type mice can be seen in FIG. 26 and in Table 40B.

TABLE 40B
Mean grip strength of CMT mice treated with and without AAT
Grip strength, newtons (Mean ± SD)

| | 8 weeks old | 10 weeks old | 12 weeks old |
|---|---|---|---|
| WT control | 7.14 ± 0.66 | 7.58 ± 1.40 | 7.69 ± 1.17 |
| CMT1A + vehicle | 3.34 ± 0.39 | 3.27 ± 0.32 | 2.74 ± 0.57 |
| CMT1A + AAT | 3.35 ± 0.27 | 5.47 ± 1.11 | 3.87 ± 0.36 |

The decrease of nerve CMAP amplitude is shown in FIG. 27 and in Table 40C

TABLE 40C
Mean CMAP of CMT mice treated with and without AAT
Compound muscle action potential, mV (Mean ± SD)

| | 8 weeks old | 10 weeks old | 12 weeks old |
|---|---|---|---|
| WT control | 7.55 ± 1.26 | 7.66 ± 1.15 | 7.97 ± 1.33 |
| CMT1A + vehicle | 3.72 ± 0.48 | 2.63 ± 0.56 | 1.81 ± 0.56 |
| CMT1A + AAT | 3.76 ± 0.50 | 5.41 ± 0.71 | 2.94 ± 0.65 |

and the decrease of sciatic nerve velocity is shown in FIG. 28 and in Table 40D.

TABLE 40D
Mean NCV of CMT mice treated with and without AAT
Nerve conduction velocity, m/s (Mean ± SD)

| | 8 weeks old | 10 weeks old | 12 weeks old |
|---|---|---|---|
| WT control | 32.78 ± 8.29 | 34.10 ± 5.94 | 33.81 ± 10.49 |
| CMT1A + vehicle | 16.01 ± 3.05 | 10.68 ± 4.12 | 6.71 ± 0.75 |
| CMT1A + AAT | 15.96 ± 1.54 | 21.47 ± 6.07 | 10.50 ± 1.73 |

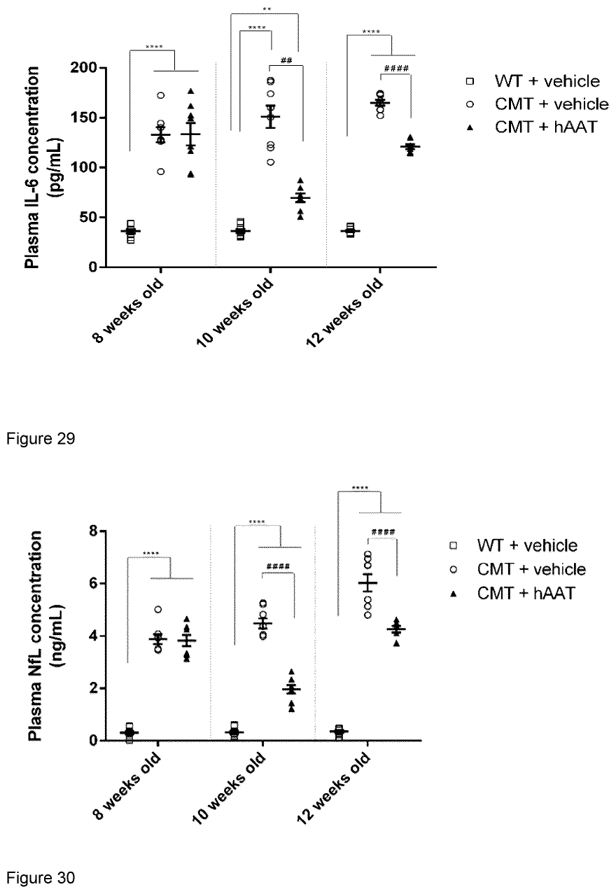

The plasma cytokine levels of IL-6 and TNF are increased in CMT mice (Table 40E, Table 40F and FIG. 29 and FIG. 30), as well as the biomarker ofnerve damage NfL (Table 40G and FIG. 40).

TABLE 40E
Mean plasma IL-6 concentration of CMT
mice treated with and without AAT
Plasma IL-6, pg/mL (Mean ± SD)

| | 8 weeks old | 10 weeks old | 12 weeks old |
|---|---|---|---|
| WT control | 36.23 ± 5.99 | 36.50 ± 5.88 | 36.42 ± 3.28 |
| CMT1A + vehicle | 132.90 ± 21.48 | 150.91 ± 31.61 | 165.13 ± 8.40 |
| CMT1A + AAT | 133.48 ± 31.45 | 69.78 ± 11.76 | 120.90 ± 6.47 |

TABLE 40F
Mean plasma TNFα concentration of CMT
mice treated with and without AAT
Plasma TNFα, pg/mL (Mean ± SD)

| | 8 weeks old | 10 weeks old | 12 weeks old |
|---|---|---|---|
| WT control | 3.46 ± 0.40 | 3.01 ± 0.54 | 3.13 ± 0.33 |
| CMT1A + vehicle | 9.73 ± 1.73 | 12.69 ± 1.24 | 14.09 ± 0.55 |
| CMT1A + AAT | 9.80 ± 2.25 | 6.55 ± 2.11 | 11.61 ± 1.00 |

TABLE 40G
Mean plasma NfL concentration of CMT
mice treated with and without AAT
Plasma NfL, ng/mL (Mean ± SD)

| | 8 weeks old | 10 weeks old | 12 weeks old |
|---|---|---|---|
| WT control | 0.31 ± 0.19 | 0.32 ± 0.19 | 0.36 ± 0.13 |
| CMT1A + vehicle | 3.88 ± 0.53 | 4.48 ± 0.54 | 6.02 ± 0.94 |
| CMT1A + AAT | 3.83 ± 0.61 | 1.96 ± 0.46 | 4.25 ± 0.36 |

TABLE 40H
Mean histological data of CMT mice treated with and without AAT
Sciatic nerve histology (Mean ± SD)

| | | Axonal | |
|---|---|---|---|
| | Axons/100 μm2 | diameter (μm) | g-ratio |
| WT control | 40.80 ± 1.90 | 5.25 ± 3.56 | 0.56 ± 0.12 |
| CMT1A + vehicle | 16.90 ± 1.20 | 2.38 ± 0.73 | 0.75 ± 0.09 |
| CMT1A + AAT | 26.30 ± 2.00 | 3.84 ± 0.25 | 0.62 ± 0.16 |

All these clinical signs/symptoms present in CMT mice were fully established as expected at 8 weeks of age. Furthermore, the signs evolved with time and became more severe at 10 weeks of age and their severity still increased at 12 weeks of age in the control CMT mice.

The treatment with AAT started at 8 weeks of age and lasted 2 weeks, until 10 weeks of age. The results show that AAT increased neuromuscular and electrophysiological performances when administered twice a day at 90 mg/kg by subcutaneous route in CMT mice. CMT mice treated with AAT showed a significant increase in rotarod latency to fall (****: P<0.0001 vs WT+vehicle; ###: P<0.001 vs CMT+vehicle, ANOVA two-way and Bonferroni test; ns: non-significant, P>0.05 FIG. 25), significant increase in grip strength (****: P<0.0001 vs WT+vehicle; #, ####: P<0.05, P<0.0001 vs CMT+vehicle, ANOVA two-way and Bonferroni test; ns: non-significant, P>0.05 FIG. 26), significant increase in compound muscle action potential amplitude (****: P<0.0001 vs WT+vehicle; #, ###: P<0.05, P<0.001 vs CMT+vehicle, ANOVA two-way and Bonferroni test; ns: non-significant, P>0.05 FIG. 27) and significant increase in nerve conduction velocity (****: P<0.0001 vs WT+vehicle; ###: P<0.001 vs CMT+vehicle, ANOVA two-way and Bonferroni test; ns: non-significant, P>0.05 FIG. 28) when compared to CMT mice not treated with AAT. AAT reduced the plasma levels of the inflammatory cytokines IL-6 (P<0.01) and TNFα (P<0.0001); (Table 40E, Table 40F, FIG. 29 and FIG. 30), as well as the level of NfL (Table 40G and FIG. 40).

At 12 weeks of age following the 2-week treatment-free period, the effect of AAT was decreased, suggesting that repeated administrations of AAT are necessary to obtain a sustained effect. At this time, the grip strength and CMAP of the AAT treated mice had decreased relative to the end of the treatment period, while remaining significantly higher than for the untreated CMT animals (P<0.05 for both parameters). The positive effect remained significant for the IL-6, TNFα and NfL plasma levels, and even after 2 weeks treatment free period, the number of axons and the size of the myelin sheath were significantly larger in the treated animals. The only parameters becoming non-significantly different from the untreated group were the rotarod latency to fall and NCV.

### CONCLUSION

AAT therapy was evaluated in a relevant mouse model of CMT disease. At baseline, at 8 weeks of age, the disease was fully established, and the animals showed clear clinical signs and symptoms of CMT1A. AAT treatment was associated with improvement of neuropathy, as shown by a significant increase in neuromuscular and electrophysiological performances as well as an increase of the number of axons and axonal diameter and decrease of g-ratio relative to untreated CMT animals. A decrease of plasma cytokines (TNFα and IL-6) and NfL biomarker was also observed in the treated animals. It has been demonstrated that AAT was able to reduce the progression of the disease. The effects of treatment with AAT were limited in time, and 2 weeks after treatment cessation, the symptoms had worsened, although not reaching the level of the untreated CMT animals.

Taken together, these data demonstrate the potential for AAT to improve CMT1A patient outcomes, by not only reducing the progression of the disease but reversing its symptoms.

### Example 22 Inhibitor Effect of AAT-Related Peptides and Peptidomimetic on the ADAM17 Activity

Biologically active immunoregulatory sites (not associated with canonical anti-protease activity) on the surface of AAT were identified by in silico methods and several peptides were derived from those immunoregulatory sites (Lior, Yotam, et al. European Journal of Medicinal Chemistry 228 (2022): 113969). The potential of two peptides (peptide 8 and peptide 9) and one peptidomimetic (peptide 14) to modulate the ADAM17 activity was investigated.

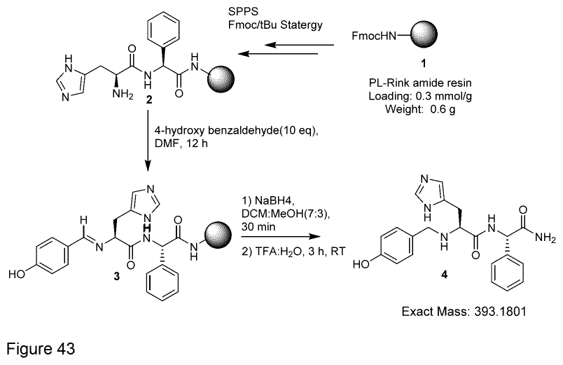

Peptide 8 (sequence: Ac-YRAHQGE-NH2; MW: 945.4) and peptide 9 (sequence: Ac-LFLYVIH-NH2; MW: 901.3) were synthesized using a Syro-1 automatic peptide synthesizer and the Fmoc/tBu strategy (FIG. 42). Peptide 14 (4-Hydroxy-Bzl-His-Phg-NH2[TFA salt]; MW: 394.2) was synthesized manually using the Fmoc/tBu strategy (FIG. 43).

ADAM-17 activity and its inhibition by human peptide 8 was measured with Recombinant Human ADAM-17 kit (Recombinant Human TACE/ADAM17 Protein, CF: 930-ADB-010 and Mca-PLAQAV-Dpa-RSSSR-NH2 Fluorogenic Peptide Substrate:ES003; R&D Systems) in black 96-well immunoplates (437111; Thermo Fisher Scientific). The enzymatic activity of ADAM-17 was measured by mixing 0.005 μg of rhADAM-17 with 10 μM of Mca-PLAQAV-Dpa-RSSSR-NH2 fluorogenic peptide substrate III in assay buffer (25 mM Tris, 2.5 μM ZnCl2, 0.005% Brij-35 (w/v), pH 9.0) to a final volume of 100 μL. Peptide 8 (Syngene) was dissolved in DMSO and diluted to the desired concentration in assay buffer. Control ADAM-17 activity was assessed in the presence of the assay buffer. Peptide 8 was added at different concentrations in 2 separate experiments (0, 10 and 50 μM in experiment 1; 0, 100, 200 and 250 μM in experiment 2) to assess its dose dependent inhibition of ADAM-17. All conditions were performed in triplicate. Activity was measured as relative fluorescent units (RFU) in endpoint mode (5 min) with a SpectraMax iD3 Microplate Reader (automatic PMT gain, wavelengths: excitation 320 nm, emission 405 nm).

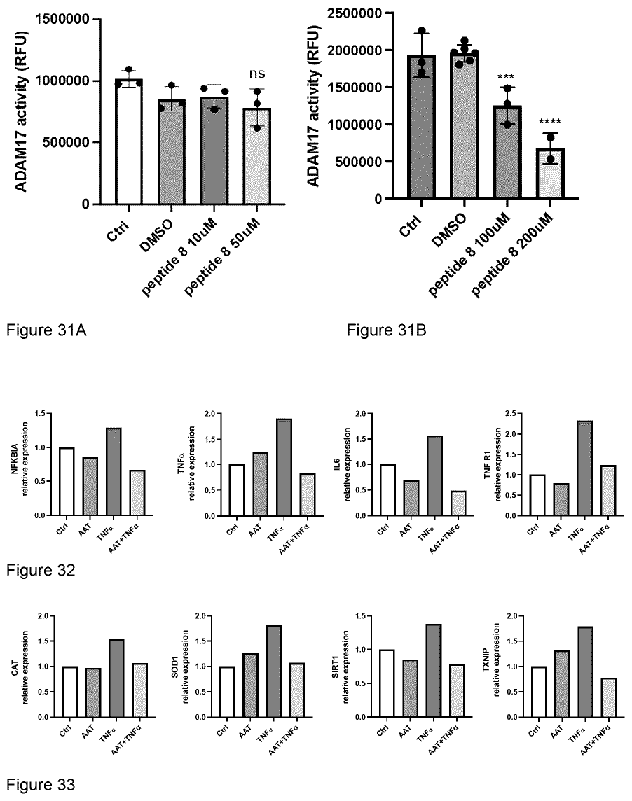

No significant inhibition of ADAM17 activity was noted at concentrations up to 50 μM (FIG. 31A left graph), while a significant concentration dependent inhibition was observed at concentrations of 100 (36% inhibition) and 200 (65% inhibition) (FIG. 31B, right graph).

### Example 23 Modulatory Effect of AAT on TNFα-Induced Schwann Cell Activation

Human Schwann cells (P10351, Innoprot) were seeded in 12 well plates at 120000 cells per well in DMEM. Cells were pre-treated with either buffer (controls) or AAT (50 μM) for 24 hours before stimulation by TNFα (10 ng/mL). The plates were then incubated at 37° C. for 24 hours. The RNA was extracted and selected target gene expression was measured by qPCR.

The rationale for gene selection is explained below.

By binding to TNFR1, TNF can activate nuclear factor κB (NF-κB), which drives cell survival signalling as well as cell death. NF-κB activation induces transcriptional upregulation of inflammasome regulators such as IL-1β, IL-18, TNFα and IFN-γ, mediating the inflammatory response. Moreover, TNFR1 can directly induce oxidative stress by the activation of reactive oxygen species (ROS) and reactive nitrogen species (RNS) producing enzymes.

The genes evaluated for their regulation included the NF-κB early gene response NFKBIA, as well as the TNFα and TNFR1 genes. Expression of the gene coding for the IL-6 pro-inflammatory cytokine was also evaluated.

The nuclear factor E2-related factor 2 (Nrf2) plays a crucial role in regulating cellular redox state in various physiological and pathological processes. Under normal physiological conditions, Nrf2 is not biologically active, nor does it activate downstream genes. Through interaction with antioxidant response elements (ARE) of cytoprotective genes, Nrf2 activates antioxidases, such as SOD, CAT, HO-1, NAD(P)H oxidase (G6PD) and others.

The T×N system plays an important role in maintaining a reduced environment in the cell. Thioredoxin (TXN) is a thiol-oxidoreductase that is a major regulator of cellular redox signaling which protects cells from oxidative stress. TXNIP interacts directly with TXN, inhibiting its ability to scavenge reactive oxygen species (ROS). It has been demonstrated that TXNIP is upregulated in diseases such as type 2 diabetes mellitus as well as neurodegenerative diseases such as Alzheimer's disease and Parkinson's disease.

As a potential early response gene, TXNIP is tightly regulated and strongly correlated with changes in mRNA levels and protein levels, and it has been shown to play an important role in the dysfunction of Schwann Cells in diabetic peripheral neuropathy.

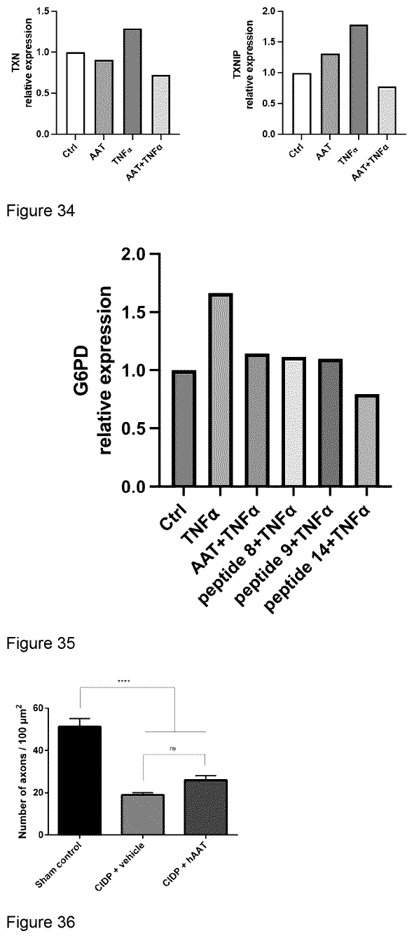

As shown in FIG. 32 to FIG. 34, the NF-κb pathway transcripts were downregulated by AAT treatment after TNF induction. AAT inhibited the expression of genes modulated by TNF-induced oxidative stress response. Taken together, these results show the modulating effects of AAT on the TNFα-induced gene expression in human Schwann cells.

### Example 24

Microglial (HMC3) cells were seeded into 96-well plates at a density of approximately 2500 cells/well. Plasma derived AAT (Sigma, 25 μM) or peptide 8, peptide 9 or peptide 14 (Syngene, 50 μM) were added, and microglial activation was induced with interferon gamma (IFNγ) (10 ng/mL) for 24 h with no preincubation. Then, the RNA was extracted (RNAeasy mini kit, Qiagen) and the expression of the Interleukin-6 (IL6) and Interleukin-Ibeta (ILIB) genes were analysed. qPCR was performed with Syber green POWER UP master mix using 10 ng/well of cDNA in all gene expression results.

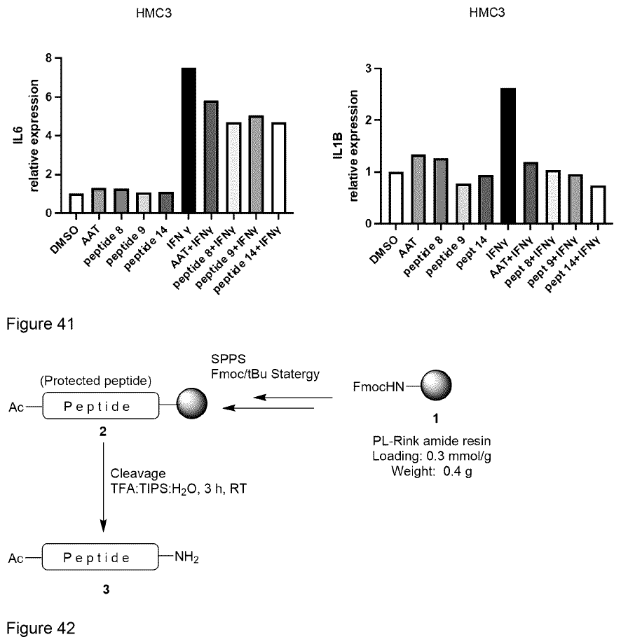

The results show that IL6 expression was reduced by 30-40% and IL1B expression was reduced by 60-70% by AAT and its derivate peptides (FIG. 41), suggesting that all three peptides mimic AAT function in an inflammatory response.

### Example 25 Effect of AAT and its Derivative Peptides on TNF Induced Schwann Cells

Schwann cells were seeded at a density of 75000 per well. At the time of seeding, plasma derived AAT (50 uM) or peptide 8,9,14 (Syngene, 50 uM) was added. The following day, cells were treated with 10 ng/ml of TNF alpha with or without AAT and its derivate peptides for 24 hours.

The NAD(P)H oxidase (G6PD) expression is induced by TNF treatment in Schwann cells and reduced by 40-30% in stimulated cells treated with AAT and its derivative peptides (FIG. 35). G6PD plays a significant role in the generation of ROS.

The invention further relate to the following items:
1. A pharmaceutical product for use in the treatment of chronic inflammatory demyelinating polyneuropathy, the pharmaceutical product comprising:
   a) alpha1-antitrypsin (AAT) protein, a variant, an isoform and/or a fragment thereof having ADAM17 inhibitory activity; and/or
   b) a nucleic acid encoding AAT, a variant, an isoform and/or a fragment thereof having ADAM17 inhibitory activity.
2. A kit of parts for use in the treatment of a disease or disorder of the nervous system, the kit comprising:
   i) a) alpha1-antitrypsin (AAT) protein, a variant, an isoform and/or a fragment thereof having ADAM17 inhibitory activity; and/or
      b) a nucleic acid encoding AAT, a variant, an isoform and/or a fragment thereof having ADAM17 inhibitory activity; and
   ii) a plurality of IgG antibodies, IgG variants, isoforms and/or fragments thereof.
3. A pharmaceutical composition for use in treatment of a disease or disorder of the nervous system, the composition comprising:
   i) a) alpha1-antitrypsin (AAT) protein, a variant, an isoform and/or a fragment thereof having ADAM17 inhibitory activity; and/or
      b) a nucleic acid encoding AAT, a variant, an isoform and/or a fragment thereof having ADAM17 inhibitory activity; and
   ii) a plurality of IgG antibodies, IgG variants, isoforms and/or fragments thereof; and
   iii) at least one pharmaceutically acceptable carrier.
4. A method of treatment comprising administering an effective amount of a pharmaceutical composition comprising AAT protein and/or a nucleic acid encoding AAT to a subject, wherein the subject is suffering from a disease or disorder of the nervous system and wherein the subject is undergoing a therapy comprising administration of a plurality of IgG antibodies, IgG variants, isoforms and/or fragments thereof.
5. A method of treatment comprising administering an effective amount of a pharmaceutical compound comprising a plurality of IgG antibodies, IgG variants, isoforms and/or fragments thereof to a subject, wherein the subject is suffering from a disease or disorder of the nervous system and wherein the subject is undergoing a therapy comprising administration of AAT protein and/or a nucleic acid encoding AAT.
6. The kit of parts for use of item 2, the pharmaceutical composition for use of item 3, the method of treatment of item 4 or 5, wherein the disease or disorder of the nervous system is chronic inflammatory demyelinating polyneuropathy.
7. The pharmaceutical product for use of item 1, the kit of parts for use of item 6, the pharmaceutical composition for use of item 6, the method of treatment of item 6, wherein a subject to be treated has at least one symptom of chronic inflammatory demyelinating polyneuropathy or a history of at least one symptom of chronic inflammatory demyelinating polyneuropathy.
8. The kit of parts for use of any one of the items 2, 6 or 7, the pharmaceutical composition for use of any one of the items 3, 6 or 7, the method of treatment of any one of the items 4 to 7, wherein the plurality of IgG antibodies, IgG variants, isoforms and/or fragments thereof are plasma derived IgG antibodies, IgG variants, isoforms and/or fragments thereof.
9. The kit of parts for use of any one of the items 2, 6 or 7, the pharmaceutical composition for use of any one of the items 3, 6 or 7, the method of treatment of any one of the items 4 to 7, wherein the plurality of IgG antibodies, IgG variants, isoforms and/or fragments thereof are recombinant IgG antibodies, IgG variants, isoforms and/or fragments thereof.
10. The kit of parts for use of any one of the items 2, 6 to 9, the pharmaceutical composition for use of any one of the items 3, 6 to 9, the method of treatment of any one of the items 4 to 9, wherein the plasma derived IgG antibodies, IgG variants, isoforms and/or fragments thereof are formulated for intravenous administration.
11. The kit of parts for use of any one of the items 2, 6 to 9, the pharmaceutical composition for use of any one of the items 3, 6 to 9, the method of treatment of any one of the items 4 to 9, wherein the plasma derived IgG antibodies, IgG variants, isoforms and/or fragments thereof are formulated for subcutaneous administration.
12. The pharmaceutical product for use of any one of the items 1 or 7, the kit of parts for use of any one of the items 2, 6 to 11, the pharmaceutical composition for use of any one of the items 3, 6 to 11, the method of treatment of any one of the items 4 to 11, wherein the AAT protein is recombinant AAT.
13. The pharmaceutical product for use of any one of the items 1 or 7, the kit of parts for use of any one of the items 2, 6 to 11, the pharmaceutical composition for use of any one of the items 3, 6 to 11, the method of treatment of any one of the items 4 to 11, wherein the AAT protein is plasma derived AAT.
14. The pharmaceutical product for use of any one of the items 1, 7, 12 or 13, the kit of parts for use of any one of the items 2, 6 to 13, the pharmaceutical composition for use of any one of the items 3, 6 to 13, the method of treatment of any one of the items 4 to 13, wherein
   a) the alpha1-antitrypsin (AAT) protein, variant, isoform and/or a fragment thereof having ADAM17 inhibitory activity; and/or
   b) the nucleic acid encoding AAT, variant, isoform and/or fragment thereof having ADAM17 inhibitory activity
is/are formulated for intravenous administration.
15. The pharmaceutical product for use of any one of the items 1, 7, 12 or 13, the kit of parts for use of any one of the items 2, 6 to 13, the pharmaceutical composition for use of any one of the items 3, 6 to 13, the method of treatment of any one of the items 4 to 13, wherein
   a) the alpha1-antitrypsin (AAT) protein, variant, isoform and/or a fragment thereof having ADAM17 inhibitory activity; and/or
   b) the nucleic acid encoding AAT, variant, isoform and/or fragment thereof having ADAM17 inhibitory activity
is/are formulated for subcutaneous administration.

SEQ ID NO: 1:
MPSSVSWGILLLAGLCCLVPVSLAEDPQGDAAQKTDTSHHDQDHPTFNKI
TPNLAEFAFSLYRQLAHQSNSTNIFFSPVSIATAFAMLSLGTKADTHDEI
LEGLNFNLTEIPEAQIHEGFQELLRTLNQPDSQLQLTTGNGLFLSEGLKL
VDKFLEDVKKLYHSEAFTVNFGDTEEAKKQINDYVEKGTQGKIVDLVKEL
DRDTVFALVNYIFFKGKWERPFEVKDTEEEDFHVDQVTTVKVPMMKRLGM
FNIQHCKKLSSWVLLMKYLGNATAIFFLPDEGKLQHLENELTHDIITKFL
ENEDRRSASLHLPKLSITGTYDLKSVLGQLGITKVFSNGADLSGVTEEAP
LKLSKAVHKAVLTIDEKGTEAAGAMFLEAIPMSIPPEVKFNKPFVFLMIE
QNTKSPLFMGKVVNPTQK
SEQ ID NO: 2:
VFALVNYIFFKGKWERPFEVKDTEEEDFHVDQVTTVKVPMMKRLGMFNIQ
HCKKLSSWVLLMKYLGNATAIFFLPDEGKLQHLENELTHDIITKFLENED
RRSASLHLPKLSITGTYDLKSVLGQLGITKVFSNGADLSGVTEEAPLKLS
KAVHKAVLTIDEKGTEAAGAMFLEAIPMSIPPEVKFNKPFVFLMIEQNTK
SPLFMGKVVNPTQK

1. A method of treating an inflammatory disease or disorder, the method comprising administering an effective amount of a pharmaceutical product to a subject, wherein the pharmaceutical product comprises:
a) alpha1-antitrypsin (AAT) protein, a variant, an isoform and/or a fragment thereof, wherein said variant, isoform and/or fragment has ADAM17 inhibitory activity or a small molecule having ADAM17 inhibitory activity; and/or
b) a nucleic acid encoding AAT, a variant, an isoform and/or a fragment thereof, wherein said variant, isoform and/or fragment has inhibitory activity.

2. The method of claim 1, wherein the inflammatory disease or disorder is an autoimmune inflammatory disease.

3. The method of claim 1, wherein the inflammatory disease or disorder is an inflammatory disease or disorder of the nervous system, preferably wherein the inflammatory disease or disorder is neuropathic pain.

4. The method of claim 1, wherein the inflammatory disease or disorder is chronic inflammatory demyelinating polyneuropathy.

5. The method of claim 1, wherein the inflammatory disease or disorder is complex regional pain syndrome.

6. The method of claim 1, wherein the inflammatory disease or disorder is inflammatory pain.

7.-8. (canceled)

9. A method of treatment comprising administering an effective amount of a pharmaceutical composition to a subject, wherein the subject is undergoing a therapy comprising administration of a plurality of IgG antibodies, isoforms thereof, fragments thereof, and/or IgG variants, or
administering an effective amount of a pharmaceutical compound comprising a plurality of IgG antibodies, isoforms thereof, fragments thereof, and/or IgG variants to a subject, wherein the subject is suffering from a disease or disorder of the nervous system, wherein the pharmaceutical composition comprises:
a) AAT protein, a variant, an isoform and/or a fragment thereof, wherein said variant, isoform and/or fragment has inhibitory activity or a small molecule having ADAM17 inhibitory activity; and/or
b) a nucleic acid encoding AAT, a variant, an isoform and/or a fragment thereof, wherein said variant, isoform and/or fragment has inhibitory activity,
wherein the subject is suffering from a disease or disorder of the nervous system.

10. (canceled)

11. The method of treatment of claim 9, wherein the disease or disorder of the nervous system is pain caused by a disease or disorder of the nervous system, preferably chronic pain caused by a disease or disorder of the nervous system.

12. The method of treatment of claim 9, wherein the disease or disorder of the nervous system is an autoimmune disease or disorder of the nervous system.

13. The method of treatment of claim 12, wherein the disease or disorder of the nervous system, preferably the inflammatory disease or disorder of the nervous system, is chronic inflammatory demyelinating polyneuropathy.

14. The method of treatment of claim 13, wherein a subject to be treated has at least one symptom of chronic inflammatory demyelinating polyneuropathy or a history of at least one symptom of chronic inflammatory demyelinating polyneuropathy.

15. The method of treatment of claim 9, wherein the plurality of IgG antibodies, isoforms thereof, fragments thereof, and/or IgG variants are plasma derived IgG antibodies, isoforms thereof, fragments thereof, and/or IgG variants.

16. The method of treatment of claim 9, wherein the plurality of IgG antibodies, isoforms thereof, fragments thereof, and/or IgG variants are recombinant IgG antibodies, isoforms thereof, fragments thereof, and/or IgG variants.

17. The method of treatment of claim 9, wherein the plasma derived IgG antibodies, isoforms thereof, fragments thereof, and/or IgG variants are formulated for intravenous administration.

18. The method of treatment of claim 9, wherein the plasma derived IgG antibodies, isoforms thereof, fragments thereof, and/or IgG variants are formulated for subcutaneous administration.

19. The method of claim 1, wherein the AAT protein is recombinant AAT.

20. The method of claim 1, wherein the AAT protein is plasma derived AAT.

21. The method of claim 1, wherein
a) alpha1-antitrypsin (AAT) protein, a variant, an isoform and/or a fragment thereof, wherein said variant, isoform and/or fragment has inhibitory activity or a small molecule having ADAM17 inhibitory activity; and/or
b) a nucleic acid encoding AAT, a variant, an isoform and/or a fragment thereof,
wherein said variant, isoform and/or fragment has inhibitory activity, is/are formulated for intravenous administration.

22. The method of claim 1, wherein
a) alpha1-antitrypsin (AAT) protein, a variant, an isoform and/or a fragment thereof, wherein said variant, isoform and/or fragment has inhibitory activity or a small molecule having ADAM17 inhibitory activity; and/or
b) a nucleic acid encoding AAT, a variant, an isoform and/or a fragment thereof,
wherein said variant, isoform and/or fragment has inhibitory activity. is/are formulated for subcutaneous administration.


SEQUENCE LISTING (5 sequences)

SEQ ID NO: 1 - protein, 418 aa, Homo sapiens
MPSSVSWGILLLAGLCCLVPVSLAEDPQGDAAQKTDTSHHDQDHPTFNKITPNLAEFAFSLYRQLAHQSNSTNIFFSPVSIATAFAMLSLGTKADTHDEILEGLNFNLTEIPEAQIHEGFQELLRTLNQPDSQLQLTTGNGLFLSEGLKLVDKFLEDVKKLYHSEAFTVNFGDTEEAKKQINDYVEKGTQGKIVDLVKELDRDTVFALVNYIFFKGKWERPFEVKDTEEEDFHVDQVTTVKVPMMKRLGMFNIQHCKKLSSWVLLMKYLGNATAIFFLPDEGKLQHLENELTHDIITKFLENEDRRSASLHLPKLSITGTYDLKSVLGQLGITKVFSNGADLSGVTEEAPLKLSKAVHKAVLTIDEKGTEAAGAMFLEAIPMSIPPEVKFNKPFVFLMIEQNTKSPLFMGKVVNPTQK

SEQ ID NO: 2 - protein, 214 aa, Homo sapiens
VFALVNYIFFKGKWERPFEVKDTEEEDFHVDQVTTVKVPMMKRLGMFNIQHCKKLSSWVLLMKYLGNATAIFFLPDEGKLQHLENELTHDIITKFLENEDRRSASLHLPKLSITGTYDLKSVLGQLGITKVFSNGADLSGVTEEAPLKLSKAVHKAVLTIDEKGTEAAGAMFLEAIPMSIPPEVKFNKPFVFLMIEQNTKSPLFMGKVVNPTQK

SEQ ID NO: 3 - protein, 17 aa, synthetic construct
  MOD_RES 11: note = X is Norleucine
  MOD_RES 1: note = V is N-acetylated
  MOD_RES 17: note = C-terminal COOH-group is turned into CONH2
VKFNKPFVFLXIEQNTK

SEQ ID NO: 4 - protein, 7 aa, synthetic construct
  MOD_RES 1: note = Y is N-acetylated
  MOD_RES 7: note = C-terminal COOH-group is turned into CONH2
YRAHQGE

SEQ ID NO: 5 - protein, 7 aa, synthetic construct
  MOD_RES 1: note = L is N-acetylated
  MOD_RES 7: note = C-terminal COOH-group is turned into CONH2
LFLYVIH